(19) United States
(12) Patent Application Publication
(10) Pub. No.: US 2020/0121756 A1
(43) Pub. Date: Apr. 23, 2020

(54) COMPOSITIONS COMPRISING BACTERIAL STRAINS

(71) Applicant: 4D Pharma Research Limited, Aberdeen (GB)

(72) Inventors: Helene Myriam SAVIGNAC, Aberdeen (GB); Imke Elisabeth MULDER, Aberdeen (GB); Ted DINAN, County Cork (IE); John CRYAN, County Cork (IE)

(21) Appl. No.: 16/692,734

(22) Filed: Nov. 22, 2019

Related U.S. Application Data
(63) Continuation of application No. PCT/GB2018/051390, filed on May 22, 2018.

(30) Foreign Application Priority Data
May 22, 2017 (GB) 1708182.9

(51) Int. Cl. A61K 38/16; A61K 35/74

(52) U.S. Cl. CPC A61K 38/164; C12R 1/46; A61K 35/74

(57) ABSTRACT

The invention provides compositions comprising bacterial strains for treating and preventing central nervous system disorders and conditions.

### CROSS-REFERENCE

This application is a continuation of International Application No. PCT/GB2018/051390, filed May 22, 2018, which claims the benefit of Great Britain Application No. 1708182.9, filed May 22, 2017, all of which are hereby incorporated by reference in their entirety.

### SEQUENCE LISTING

The instant application contains a Sequence Listing which has been submitted electronically in ANSI format and is hereby incorporated by reference in its entirety. Said ANSI copy, created on Nov. 21, 2019, is named 56708_726_301 SL and is 3,846,144 bytes in size.

### TECHNICAL FIELD

This invention is in the field of compositions comprising bacterial strains isolated from the mammalian digestive tract and the use of such compositions in the treatment of disease.

### BACKGROUND TO THE INVENTION

The human intestine is thought to be sterile in utero, but it is exposed to a large variety of maternal and environmental microbes immediately after birth. Thereafter, a dynamic period of microbial colonization and succession occurs, which is influenced by factors such as delivery mode, environment, diet and host genotype, all of which impact upon the composition of the gut microbiota, particularly during early life. Subsequently, the microbiota stabilizes and becomes adult-like [1]. The human gut microbiota contains more than 500-1000 different phylotypes belonging essentially to two major bacterial divisions, the Bacteroidetes and the Firmicutes [2]. The successful symbiotic relationships arising from bacterial colonization of the human gut have yielded a wide variety of metabolic, structural, protective and other beneficial functions. The enhanced metabolic activities of the colonized gut ensure that otherwise indigestible dietary components are degraded with release of by-products providing an important nutrient source for the host. Similarly, the immunological importance of the gut microbiota is well-recognized and is exemplified in germfree animals which have an impaired immune system that is functionally reconstituted following the introduction of commensal bacteria [3-5].

The discovery of the size and complexity of the human microbiome has resulted in an on-going evaluation of many concepts of health and disease. Certainly, dramatic changes in microbiota composition have been documented in gastrointestinal disorders such as inflammatory bowel disease (IBD)[6-9]. More recently, there is increased interest in the art regarding alternations in the gut microbiome that may play a pathophysiological role in human brain diseases [10]. Preclinical and clinical evidence are strongly suggesting a link between brain development and microbiota [11].

A growing body of preclinical literature has demonstrated bidirectional signalling between the brain and the gut microbiome, involving multiple neurocrine and endocrine signalling systems. Indeed, increased levels of Clostridium species in the microbiome have been linked to brain disorders [12], and an imbalance of the Bacteroidetes and Firmicutes phyla has also been implicated in brain development disorders [13]. Suggestions that altered levels of gut commensals, including those of Bifidobacterium, Lactobacillus, Sutterella, Prevotella and Ruminococcus genera and of the Alcaligenaceae family are involved in immune-mediated central nervous system (CNS) disorders, are questioned by studies suggesting a lack of alteration in the microbiota between patients and healthy subjects [14]. This indicates that, at present, the practical effect of the link between the microbiome and human brain diseases is poorly characterised. Accordingly, more direct analytical studies are required to identify the therapeutic impact of altering the microbiome on CNS disorders.

In recognition of the potential positive effect that certain bacterial strains may have on the animal gut, various strains have been proposed for use in the treatment of various diseases (see, for example, [14-17]). Also, certain strains, including mostly Lactobacillus and Bifidobacterium strains, have been proposed for use in treating various inflammatory and autoimmune diseases that are not directly linked to the intestines (see [18] and [19] for reviews). In addition, a range of probiotics have been investigated in animal models to determine a role of the gut microbiome in modulating emotional behaviour, and Bifidobacterium and Lactobacillus are the main genera showing beneficial effects, reducing anxiety and repetitive behaviours, and increasing social interaction [20-22]. However, the relationship between different diseases and different bacterial strains, and the precise effects of particular bacterial strains on the gut and at a systemic level and on any particular types of diseases, are poorly characterised, particularly for central nervous system diseases.

There is a growing body of evidence to suggest that the microbiota-gut-brain axis is affected in autism spectrum disorders (ASD) and other neurodevelopmental and neuropsychiatric disorders. Animal models have provided considerable insight into how the microbiota may be involved in ASD. Furthermore, preclinical studies have demonstrated that targeting the gut microbiota through administration of beneficial live biotherapeutics display efficacy in improving autistic-related behaviour in animal models, including the maternal immune activation (MIA) mouse model and the black and tan, brachyuric (BTBR) mouse. The BTBR mouse is a genetically modified, inbred mouse strain that displays a number of behaviours associated with ASD such as impaired sociability, repetitive behaviour and increased anxiety. Moreover, these mice also exhibit gastrointestinal dysfunctions along with alterations to the composition of the gut microbiota. Consequently, it represents an appropriate animal model for investigating the role of the microbiota-gut-brain axis in ASD.

Reference [23] discusses possible methods of treating neurodevelopmental disorders by administering a composition comprising a bacterial species selected from Bacteroides and/or Enterococcus, but provides data only for Bacteroides. Reference [24] discusses a similar use of Bacteroides and Enterococcus, with data limited to Bacteroides fragilis, Bacteroides vulgatus and Enterococcus faecalis.

Enterococcus faecalis and Enterococcus faecium are phenotypically and genetically distinct bacterial species within the Enterococcus genus. Enterococcus faecalis and Enterococcus faecium are also phylogenetically distant. Enterococcus faecalis strains display traits related to overt virulence while Enterococcus faecium is a species virtually devoid of known overt pathogenic traits 25].

Accordingly, there is a requirement in the art for new methods of treating central nervous system disorders. There is also a requirement for the potential effects of gut bacteria to be characterised so that new therapies using gut bacteria can be developed.

### SUMMARY OF THE INVENTION

The inventors have developed new therapies for treating and preventing central nervous system disorders. In particular, the inventors have developed new therapies for treating and preventing central nervous system disorders and conditions mediated by the microbiota-gut-brain axis. In particular, the inventors have identified that bacterial strains of the species Enterococcus faecium can be effective for treating and preventing diseases and conditions mediated by the microbiota-gut-brain axis. As described in the examples, oral administration of compositions comprising Enterococcus faecium may reduce symptoms associated with dysfunction of the microbiota-gut-brain axis in a mouse model of autism spectrum disorders.

Therefore, in a first embodiment, the invention provides a composition comprising a bacterial strain of the species Enterococcus faecium, for use in a method of treating or preventing a central nervous system disorder or condition. In particular embodiments, the central nervous system disorder or condition is mediated by the microbiota-gut-brain axis. In further embodiments, the invention provides a composition comprising a bacterial strain of the species Enterococcus faecium, for use in a method of treating or preventing a neurodevelopmental disorder or a neuropsychiatric condition. The inventors have identified that treatment with bacterial strains from this species can provide clinical benefits in mouse models of central nervous system disorders, in particular those mediated by the microbiota-gut-brain axis. The inventors have identified that treatment with bacterial strains from this species may modulate signalling in the central, autonomic and enteric nervous systems; may modulate the activity of the hypothalamus-pituitary-adrenal (HPA) axis pathway; may modulate neuroendocrine and/or neuroimmune pathways; and/or may modulate the levels of commensal metabolites, inflammatory markers and/or gastrointestinal permeability of a subject.

In particular embodiments, the invention provides a composition comprising a bacterial strain of the species Enterococcus faecium, for use in a method of treating or preventing a disease or condition selected from the group consisting of: autism spectrum disorders (ASDs); child developmental disorder; obsessive compulsive disorder (OCD); major depressive disorder; depression; seasonal affective disorder; anxiety disorders; chronic fatigue syndrome (myalgic encephalomyelitis); stress disorder; post-traumatic stress disorder; schizophrenia spectrum disorders; schizophrenia; bipolar disorder; psychosis; mood disorder; dementia; Alzheimer's; Parkinson's disease; and/or chronic pain. In further embodiments, the compositions of the invention may be useful for treating or preventing motor neuron disease; Huntington's disease; Guillain-Barre syndrome and/or meningitis. The effect shown for the bacterial strains from the species Enterococcus faecium on the microbiota-gut-brain axis and on diseases mediated by the microbiota-gut-brain axis may provide therapeutic benefits for other diseases and conditions mediated by the microbiota-gut-brain axis, such as those listed above. In other embodiments, the invention provides a composition comprising a bacterial strain of the species Enterococcus faecium, for use in a method of treating comorbidities associated with diseases and conditions mediated by the microbiota-gut-brain axis, such as those listed above. In particularly preferred embodiments, the invention provides a composition comprising a bacterial strain of the species Enterococcus faecium, for use in a method of treating gastrointestinal comorbidities associated with diseases and conditions mediated by the microbiota-gut-brain axis, such as those listed above. The mouse model experiments used in this application for the assessment of the symptoms of autism spectrum disorders are known in the art to be applicable for the assessment of the symptoms other central nervous system disorders including those listed above [26- 27].

In particularly preferred embodiments, the invention provides a composition comprising a bacterial strain of the species Enterococcus faecium, for use in a method of treating or preventing autism spectrum disorders, such as autism. The inventors have identified that treatment with Enterococcus faecium strains can reduce symptom severity in a mouse model of autism spectrum disorders and can prevent or reduce stereotyped, repetitive, compulsive and anxious behaviour. In preferred embodiments, the invention provides a composition comprising a bacterial strain of the species Enterococcus faecium, for use in the treatment of autism spectrum disorders. Compositions using Enterococcus faecium may be particularly effective for treating autism spectrum disorders. In preferred embodiments, the invention provides a composition for use in reducing stereotyped, repetitive, compulsive or anxious behaviour, in particular in the treatment of autism spectrum disorders. In preferred embodiments, the invention provides a composition comprising a bacterial strain of the species Enterococcus faecium, for use in the treatment of the behavioural symptoms of autism spectrum disorders. In preferred embodiments, the invention provides a composition comprising a bacterial strain of the species Enterococcus faecium for use in the treatment of the gastrointestinal symptoms of autism spectrum disorders. In preferred embodiments, the invention provides a composition comprising a bacterial strain of the species Enterococcus faecium, for use in the treatment of the behavioural and gastrointestinal symptoms of autism spectrum disorders. Treatment with Enterococcus faecium strains may modulate signalling in the central, autonomic and enteric nervous systems; may modulate the activity of the HPA axis pathway; may modulate neuroendocrine and/or neuroimmune pathways; and/or may modulate the levels of commensal metabolites, inflammatory markers and/or gastrointestinal permeability of a subject, all of which are implicated in the neuropathology of autism spectrum disorders. In certain embodiments, treatment with Enterococcus faecium strains may modulate the levels of oxytocin and/or vasopressin hormones.

In further preferred embodiments, the invention provides a composition comprising a bacterial strain of the species Enterococcus faecium, for use in a method of treating or preventing obsessive compulsive disorder (OCD). In preferred embodiments, the invention provides a composition for use in reducing stereotyped, repetitive, compulsive or anxious behaviour in the treatment of OCD. Treatment with Enterococcus faecium strains may modulate signalling in the central, autonomic and enteric nervous systems; may modulate the activity of the HPA axis pathway; may modulate neuroendocrine and/or neuroimmune pathways; and/or may modulate the levels of commensal metabolites and/or gastrointestinal permeability of a subj ect, all of which are implicated in the neuropathology of OCD.

In further preferred embodiments, the invention provides a composition comprising a bacterial strain of the species Enterococcus faecium, for use in a method of treating or preventing major depressive disorder (MDD). Treatment with Enterococcus faecium strains may provide clinical benefits in a mouse model of depression. In preferred embodiments, the invention provides a composition comprising a bacterial strain of the species Enterococcus faecium, for use in the treatment of depression. Compositions using Enterococcus faecium may be particularly effective for treating depression. In preferred embodiments, the invention provides a composition for use in reducing stereotyped, repetitive, compulsive or anxious behaviour in the treatment of depression. Treatment with Enterococcus faecium strains may modulate signalling in the central, autonomic and enteric nervous systems; may modulate the activity of the HPA axis pathway; may modulate neuroendocrine and/or neuroimmune pathways; and may modulate the levels of commensal metabolites, inflammatory markers and/or gastrointestinal permeability of a subject, all of which are implicated in the neuropathology of MDD. In certain embodiments, treatment with Enterococcus faecium strains may modulate the levels of oxytocin and/or vasopressin hormones.

In further preferred embodiments, the invention provides a composition comprising a bacterial strain of the species Enterococcus faecium, for use in a method of treating or preventing anxiety disorders. Treatment with Enterococcus faecium strains reduces disease incidence and disease severity in a mouse model of anxiety in the examples of this application. In preferred embodiments, the invention provides a composition comprising a bacterial strain of the species Enterococcus faecium, for use in the treatment of anxiety disorder. Compositions using Enterococcus faecium may be particularly effective for treating anxiety disorder. In preferred embodiments, the invention provides a composition for use in reducing stereotyped, repetitive, compulsive or anxious behaviour in the treatment of anxiety.

In further preferred embodiments, the invention provides a composition comprising a bacterial strain of the species Enterococcus faecium, for use in a method of treating or preventing stress disorders, such as post-traumatic stress disorder. Compositions comprising a bacterial strain of the species Enterococcus faecium may reduce stress in mouse models of stress disorders. Treatment with Enterococcus faecium strains may modulate signalling in the central, autonomic and enteric nervous systems; may modulate the activity of the HPA axis pathway; may modulate neuroendocrine and/or neuroimmune pathways; and may modulate the levels of commensal metabolites, inflammatory markers and/or gastrointestinal permeability of a subject, all of which are implicated in the neuropathology of stress disorder. In certain embodiments, treatment with Enterococcus faecium strains may modulate the levels of oxytocin and/or vasopressin hormones.

In further preferred embodiments, the invention provides a composition comprising a bacterial strain of the species Enterococcus faecium, for use in a method of treating or preventing schizophrenia spectrum and psychotic disorders, such as schizophrenia. Compositions comprising a bacterial strain of the species Enterococcus faecium may improve positive and negative symptoms in mouse models of schizophrenia spectrum and psychotic disorders. Treatment with Enterococcus faecium strains may modulate signalling in the central, autonomic and enteric nervous systems; may modulate the activity of the HPA axis pathway; may modulate neuroendocrine and/or neuroimmune pathways; and may modulate the levels of commensal metabolites and/or gastrointestinal permeability of a subject, all of which are implicated in the neuropathology of schizophrenia spectrum and psychotic disorders.

In further preferred embodiments, the invention provides a composition comprising a bacterial strain of the species Enterococcus faecium, for use in a method of treating or preventing bipolar disorder. Compositions comprising a bacterial strain of the species Enterococcus faecium may reduce occasions of mania and/or depression in mouse models of bipolar disorder. Treatment with Enterococcus faecium strains may modulate signalling in the central, autonomic and enteric nervous systems; may modulate the activity of the HPA axis pathway; may modulate neuroendocrine and/or neuroimmune pathways; and may modulate the levels of commensal metabolites, inflammatory markers and/or gastrointestinal permeability of a subject, all of which are implicated in the neuropathology of bipolar disorder. In certain embodiments, treatment with Enterococcus faecium strains may modulate the levels of oxytocin and/or vasopressin hormones.

In further preferred embodiments, the invention provides a composition comprising a bacterial strain of the species Enterococcus faecium, for use in a method of treating or preventing neurocognitive disorders, such as Alzheimer's disease. Compositions comprising a bacterial strain of the species Enterococcus faecium may improve cognitive and behavioural functioning in mouse models of neurocognitive disorders. Treatment with Enterococcus faecium strains may modulate signalling in the central, autonomic and enteric nervous systems; may modulate the activity of the HPA axis pathway; may modulate neuroendocrine and/or neuroimmune pathways; and may modulate the levels of commensal metabolites and/or gastrointestinal permeability of a subject, all of which are implicated in the neuropathology of neurocognitive disorders.

In further preferred embodiments, the invention provides a composition comprising a bacterial strain of the species Enterococcus faecium, for use in a method of treating or preventing Parkinson's disease. Compositions comprising a bacterial strain of the species Enterococcus faecium may improve motor and cognitive functions in mouse models of Parkinson's disease. Treatment with Enterococcus faecium strains may modulate signalling in the central, autonomic and enteric nervous systems; may modulate the activity of the HPA axis pathway; may modulate neuroendocrine and/or neuroimmune pathways; and may modulate the levels of commensal metabolites, inflammatory markers and/or gastrointestinal permeability of a subject, all of which are implicated in the neuropathology of Parkinson's disease. In certain embodiments, treatment with Enterococcus faecium strains may modulate the levels of oxytocin and/or vasopressin hormones.

In certain embodiments, the compositions of the invention are for use in a method of modulating the microbiota-gut-brain axis in the treatment or prevention of a disease or condition mediated by the microbiota-gut-brain axis. In particular, the compositions of the invention may be used in modulating the microbiota-gut-brain axis in the treatment or prevention of autism spectrum disorders; obsessive compulsive disorder; major depressive disorder; anxiety disorders; stress disorders; schizophrenia spectrum disorders; bipolar disorders; neurocognitive disorders and Parkinson's disease.

In preferred embodiments of the invention, the bacterial strain in the composition is of Enterococcus faecium. Closely related strains may also be used, such as bacterial strains that have a 16s rRNA sequence that is at least 95%, 96%, 97%, 98%, 99%, 99.5% or 99.9% identical to the 16s rRNA sequence of a bacterial strain of Enterococcus faecium. Preferably, the bacterial strain has a 16s rRNA sequence that is at least 95%, 96%, 97%, 98%, 99%, 99.5% or 99.9% identical to SEQ ID NO:1 or 2. Preferably, the sequence identity is to SEQ ID NO:2. Preferably, the bacterial strain for use in the invention has the 16s rRNA sequence represented by SEQ ID NO:2.

In certain embodiments, the composition of the invention is for oral administration. Oral administration of the strains of the invention can be effective for treating central nervous system disorders and conditions, in particular those mediated by the microbiota-gut-brain axis. Also, oral administration is convenient for patients and practitioners and allows delivery to and/or partial or total colonisation of the intestine.

In certain embodiments, the composition of the invention comprises one or more pharmaceutically acceptable excipients or carriers.

In certain embodiments, the composition of the invention comprises a bacterial strain that has been lyophilised. Lyophilisation is an effective and convenient technique for preparing stable compositions that allow delivery of bacteria.

In certain embodiments, the invention provides a food product comprising the composition as described above.

In certain embodiments, the invention provides a vaccine composition comprising the composition as described above.

Additionally, the invention provides a method of treating or preventing a disease or condition mediated by dysfunction of the microbiota-gut-brain axis, comprising administering a composition comprising a bacterial strain of the species Enterococcus faecium.

In developing the above invention, the inventors have identified and characterised a bacterial strain that is particularly useful for therapy. The Enterococcus faecium strain of the invention is shown to be effective for treating the diseases described herein, such as autism spectrum disorder. Therefore, in another aspect, the invention provides a cell of the Enterococcus faecium strain deposited under accession number NCIMB 42487, or a derivative thereof. The invention also provides compositions comprising such cells, or biologically pure cultures of such cells. The invention also provides a cell of the Enterococcus faecium strain deposited under accession number NCIMB 42487, or a derivative thereof, for use in therapy, in particular for the diseases described herein.

In especially preferred embodiments, the invention provides a composition comprising the strain deposited under accession number NCIMB 42487, for use in a method of treating or preventing a central nervous system disorder or condition. In especially preferred embodiments, the invention provides a composition comprising the strain deposited under accession number NCIMB 42487, for use in a method of treating or preventing a neurodevelopmental disorder or a neuropsychiatric condition. In especially preferred embodiments, the invention provides a composition comprising the strain deposited under accession number NCIMB 42487, for use in a method of treating or preventing autism spectrum disorder, or preferably autism. In especially preferred embodiments, the invention provides a composition comprising the strain deposited under accession number NCIMB 42487, for use in a method of reducing stereotyped, repetitive, compulsive or anxious behaviour, especially in the treatment of autism.

### BRIEF DESCRIPTION OF DRAWINGS

FIGS. 1A-1B: Treatment with MRX010 decreased the number of marbles buried in MIA mouse model. #p<0.01 relative to control group. **p<0.01 relative to vehicle group. ***p<0.001 relative to vehicle group as revealed by a priori comparisons. Student's t-test analysis between the control group and the vehicle MIA group revealed that the vehicle MIA mice buried more marbles compared to the control group (t(19)=3.00, P=0.007; FIG. 1A). ANOVA of the number of marbles buried revealed an effect of treatment [F(3,42)=6.37, P=0.001]. Post-hoc tests revealed that chronic treatment with Mrx0010 decreased the number of marbles buried (p<0.01; FIG. 1A). A priori pairwise comparisons revealed that MIA mice treated with Mrx010 buried less marbles than MIA vehicle mice (p<0.001; FIG. 1B)

FIGS. 2A-2B: Effect of treatment with either vehicle or MRX010 on social transmission food preference. ANOVA of demonstrator cued food preference revealed no significant difference when observers were exposed to food choice immediately after demonstrator interaction (T0) (F(3,34)=0.38, P=0.77; FIG. 2A) or 24 hrs later (F(3,34)=0.85, P=0.48; FIG. 2B), irrespective of vehicle or MRX010 administration.

FIG. 3: Effect of treatment with either vehicle or MRX010 on immobility time of mice in the forced swimming test.

FIG. 4: Effect of treatment with MRX010 on intestinal permeability in MIA mice.

FIG. 5: Effects of chronic treatment with MRX010 on intestinal motility. ##p<0.01 relative to control group.

FIGS. 6A-6B: Effect of treatment with MRX010 on social transmission of food preference in BTBR mice. ANOVA of demonstrator cued food preference revealed no significant difference when observers were exposed to food choice immediately after demonstrator interaction (T0) (F(3,36)=1.123; P=0.354; FIG. 6A) or 24 hrs later (F(3,38)=0.138; P=0.936; FIG. 6B).

FIG. 7: Effect of treatment with MRX010 on social behaviour of BTBR mice in the social interaction test.

FIG. 8: Effect of treatment with MRX010 on stereotyped behaviour in BTBR mice in the marble burying test.

FIGS. 9A-9D: Effect of treatment with MRX010 on anxiety-like behaviour in the elevated plus maze in BTBR mice. ANOVA of percentage time spent in closed arms revealed no effect of treatment [F(3,39)=0.556; P=0.647; FIG. 9A]. Kruskal Wallis non-parametric analysis of percentage time spent in open arms [Chi squared: 10.831; df=3; P=0.013; FIG. 9B] followed by non-parametric Mann-Whitney U test revealed that mice treated with MRX010 spent no more time in the open arms compared to the vehicle group. ANOVA of the number of entries into the closed arms revealed no effect of treatment [F(3,39)=0.556; P=0.647; FIG. 9C]. Kruskal Wallis non-parametric analysis of number of the entries into the open arms [chi-squared=10.315; df=3; P=0.016; FIG. 9D] followed by non-parametric Mann-Whitney U test revealed no effect of treatment on the number of entries in the open arms.

FIGS. 10A-10C: Effect of treatment with MRX010 on locomotor activity and anxiety-like behaviour in the open field arena in BTBR mice. ANOVA of distance moved did not reveal an effect of treatment upon locomotor activity in the open field arena [F(3,37)=1.325; P=0.282, FIG. 10A]. ANOVA of time spent in the outer zone did not reveal an effect of treatment [F(3,37)=1.598; P=0.208; FIG. 10B]. ANOVA of time spent in the inner zone did reveal an effect of treatment [F(3,36)=3.636; P=0.023; FIG. 10C].

FIG. 11: Effect of treatment with MRX010 on immobility time of BTBR mice in the forced swimming test.

FIG. 12: Effect of treatment with MRX010 on upon time spent sniffing urine in BTBR mice. ##p<0.01 relative to water vehicle group.

FIG. 13: Effect of treatment with MRX010 on intestinal motility in BTBR mice.

FIGS. 14A-14D: Effect of treatment with MRX010 on selective anatomical markers in BTBR mice. *p<0.05 relative to vehicle group. ANOVA of organ weight as a percentage of body weight did not reveal an effect of treatment for the adrenals [F(3,37)=0.208; P=0.890; FIG. 14A}, spleen F(3,35)=0.629; P=0.601; FIG. 14B] or caecum [F(3,37)=0.883; P=0.460; FIG. 14C]. ANOVA of colon length revealed an effect of treatment [F(3,37)=5.635; P=0.003; FIG. 14D]. Post-hoc analysis revealed that chronic treatment with MRX010 (p<0.05 relative to the vehicle group) increased colon length in BTBR mice.

FIG. 15: Effect of treatment with MRX010 on stereotypical/anxiety-like behaviour in MIA mice in the self-grooming test. # p=0.017 relative to the control. Vehicle is phosphate buffered saline.

FIGS. 16A-16B: Effect of treatment with MRX010 on anxiety-like behaviour in MIA mice in the elevated plus maze. FIGS. 16A—open arms; FIG. 16B—closed arms. Vehicle is phosphate buffered saline.

FIG. 17: Effect of treatment with MRX010 on anxiety-like behaviour in MIA mice in the open field arena. Vehicle is phosphate buffered saline.

FIG. 18: Effect of treatment with MRX010 on social behaviour in MIA mice in the female urine sniffing test. Vehicle is phosphate buffered saline.

FIGS. 19A-19B: Effect of treatment with MRX010 on social behaviour in MIA mice in the three-chamber test. FIG. 19A—object vs mouse. * p<0.001 vs mouse. FIG. 19B—familiar mouse vs novel mouse. *p=0.005 vs control; # p=0.001 vs familiar mouse. MRX010 led to increased interaction with novel mouse and so improved social behaviour. Vehicle is phosphate buffered saline.

FIG. 20: Effect of treatment with MRX010 on cognitive performance in MIA mice in the novel object recognition test. Vehicle is phosphate buffered saline.

FIGS. 21A-21E: Effect of treatment with MRX010 on permeability and gene expression in the ileum. PBS: phosphate buffered saline; IDO-1: indoleamine-pyrrole 2,3-dioxygenase-1; TJP1: tight junction protein 1; TPH1: tryptophan hydroxylase 1. Using the passage of FITC from the luminal to the serosal side of the Ussing chamber as an index of gut permeability, it was determined that MRX010 had no observable effect on ileum (FIG. 21A (F(3,24)=0.107, p=0.96)) tissue permeability. Furthermore, MRX010 had no significant effect on mRNA expression of the tight junction proteins (involved in maintaining the integrity of the gut barrier) TJP1 (FIG. 21C (t(12)=0.16, p=0.876) or occludin (FIG. 21B (t(11)=0.72, p=0.487) in the ileum; the enzyme IDO-1(the first and rate-limiting enzyme in the tryptophan/kynurenine pathway) (FIG. 21D (t(12)=0.398, p=0.698); nor TPH1 (an isoform of the enzyme tryptophan hydroxylase, responsible for the synthesis of serotonin) (FIG. 21E ((t(12)=0.157, p=0.878) in ileum.

FIGS. 22A-22E: Effect of treatment with MRX010 on permeability and gene expression in the colon. PBS: phosphate buffered saline; IDO-1: indoleamine-pyrrole 2,3-dioxygenase-1; TJP1: tight junction protein 1; TPH1: tryptophan hydroxylase 1. Using the passage of FITC from the luminal to the serosal side of the Ussing chamber as an index of gut permeability, it was determined that MRX010 had no observable effect on colon (FIG. 22A (F(3,27)=1.141, p=0.351)) tissue permeability. Furthermore, MRX010 had no significant effect on mRNA expression of the tight junction proteins (involved in maintaining the integrity of the gut barrier) TJP1 FIG. 22D (t(8)=0.114, p=0.912)) or occludin (FIG. 22B (t(8)=0.272, p=0.972)) in the colon; the enzyme IDO-1(the first and rate-limiting enzyme in the tryptophan/kynurenine pathway) FIG. 22C (t(8)=0.51, p=0.623)); nor TPH1 (an isoform of the enzyme tryptophan hydroxylase, responsible for the synthesis of serotonin) FIG. 22E ((t(9)=0.533, p=0.611)) in colon tissue.

FIGS. 23A-23F: Effect of treatment with MRX010 on SCFA expression in caecal content. Vehicle is phosphate buffered saline. Chronic administration of MRX010 had no observable effect on the caecal production of the short chain fatty acids acetate (FIG. 23A, t(12)=1.787, p=0.099), proprionate (FIG. 23B, t(12)=1.29, p=0.222), isobutyrate (FIG. 23C, t(11)=1.152, p=0. 174), butyrate (FIG. 23D, t(12)=0.577, p=0.575), isovalearate (FIG. 23E, t(11)=1.584, p=0.142) or valearate (FIG. 23F, t(12)=0.27, p=0.292), when compared to vehicle PBS administration (FIG. 23A-F)

FIGS. 24A-24F: Effect of treatment with MRX010 on splenocyte cytokine production in response to antigen challenge, including IL-10 (FIG. 24A), IL-1β (FIG. 24B), IL-6(FIG. 24C), TNFα (FIG. 24D), CXCL1 (FIG. 24E), and IFNγ (FIG. 24F). PBS: phosphate buffered saline; LPS: lipopolysaccharide; ConA: concavalin A; IL: interleukin; TNFα: tumour necrosis factor α; CXCL1: chemokine (C-X-C motif) ligand 1; IFNγ: interferon-γ. *t(8)=2.54, p=0.035 vs PBS control.

FIGS. 25A-25T: Effect of treatment with MRX010 on plasma levels (i.e. biosynthesis and catabolism) of essential amino acids. *t(9)=2.733, p=0.023 vs PBS. MRX010 decreased proline levels in the plasma (FIG. 25F, t(9)=2.733, p=0.023), but appeared not to alter levels of tyrosine (FIG. 25A, t(12)=0.078, p=0.39), valine (FIG. 25B, t(12)=1.152, p=0.272), threonine (FIG. 25C, t(11)=0.072, p=0.944), taurine (FIG. 25D, t(12)=1.03, p=0.323), serine (FIG. 25E, t(12)=1.334, p=0.207), phenylalanine (FIG. 25G, t(12)=0.086, p=0.343), methionine (FIG. 25H, t(11)=0.564, p=0.584), lysine (FIG. 25I, t(12)=0.496, p=0.629), leucine (FIG. 25J, t(12)=0.289, p=0.778), isoleucine (FIG. 25K, t(12)=0.169, p=0.107), HN3 (FIG. 25L, t(12)=0.021, p=0.984), histidine (FIG. 25M, t(12)=0.516, p=0.615), glycine (FIG. 25N, t(12)=0.608, p=0.555), glutamate (FIG. 25O, t(12)=0.674, p=0.513), cysteic acid (FIG. 25P, t(11)=0.375, p=0.715), cysteine (FIG. 25Q, t(12)=0.718, p=0.487), aspartate (FIG. 25R, t(12)=1.009, p=0.313), arginine (FIG. 25S, t(12)=0.883, p=0.395) or alanine (FIG. 25T, t(12)=4.525, p=0.153).

FIGS. 26A-26E: Effect of treatment with MRX010 on monoamine changes in the brain. 5-HIAA: 5-hydroxy-indole-acetic acid; 5-HT: 5-hydroxy-tryptamine (serotonin). Chronic administration of MRX010 appeared not to alter levels of noradrenaline (FIG. 26A, t(12)=1.551, p=0.147), dopamine (FIG. 26B, t(12)=0.731, p=0.479), serotonin (FIG. 26C, t(12)=0.154, p=0. 149), 5-HIAA (FIG. 26D, 5-hydroxy-indole-acetic acid; a metabolite of 5-HT) (t(12)=1.858, p=0.088), or serotonin turnover (FIG. 26E, the ratio of 5-HIAA:5-HT) (t(12)=0.202, p=0.844) as determined by unpaired 2-tailed t-test.

FIGS. 27A-27F: Effect of treatment with MRX010 on hippocampal gene expression of neurotransmitter receptors. Expression of genes for neurotransmitter receptors serotonin receptor 1a(5-HT1a) (FIG. 27A), dopamine D1 receptor (FIG. 27B), GABAB receptor subunit B1 (FIG. 27C), GABAA receptor (FIG. 27D), NMDA2A (FIG. 27E) and NMDA2B receptor (FIG. 27F) were analysed in brain tissue from the hippocampus.

FIGS. 28A-28E: Effect of treatment with MRX010 on amygdala gene expression of neurotransmitter receptors. *t(11)=2.737, p=0. 019 vs PBS. Expression of genes for neurotransmitter receptors serotonin receptor 1a(5-HT1a) (FIG. 28A), dopamine D1 receptor (FIG. 28B), GABAB receptor subunit B1 (FIG. 28C), GABAA receptor (FIG. 28D), NMDA2A (FIG. 28E) and NMDA2B receptor (FIG. 28F) were analysed in brain tissue from the amygdala.

FIGS. 29A-29E: Effect of treatment with MRX010 on prefrontal cortex (PFC) gene expression of neurotransmitter receptors. Expression of genes for neurotransmitter receptors serotonin receptor 1a(5-HT1a) (FIG. 29A), dopamine D1 receptor (FIG. 29B), GABAB receptor subunit B1 (FIG. 29C), GABAA receptor (FIG. 29D), NMDA2A (FIG. 29E) and NMDA2B receptor (FIG. 29F) were analysed in brain tissue from the prefrontal cortex.

FIGS. 30A-30E: Effect of treatment with MRX010 on hippocampal gene expression of inflammatory markers. Expression of genes for inflammatory markers IL-1β (FIG. 30A), IL6 (FIG. 30B), CD11b (FIG. 30C), TNFα (FIG. 30D), and TLR4 (FIG. 30E) were analysed in brain tissue from the hippocampus.

FIGS. 31A-C: Effect of treatment with MRX010 on amygdala gene expression of inflammatory markers. Expression of genes for inflammatory markers IL-1β (FIG. 31A), IL6 (FIG. 31B), CD11b (FIG. 31C), TNFα (FIG. 31D), and TLR4 (FIG. 31E) were analysed in brain tissue from the amygdala.

FIGS. 32A-32C: Effect of treatment with MRX010 on PFC gene expression of inflammatory markers. Expression of genes for inflammatory markers IL-1β (FIG. 32A), IL6 (FIG. 32B), CD11b (FIG. 32C), TNFα (FIG. 32D), and TLR4 (FIG. 32E) were analysed in brain tissue from the prefrontal cortex.

FIGS. 33A-33H: Effect of treatment with MRX010 on hippocampal gene expression of endocrine markers. Expression of genes for endocrine markers corticosterone releasing factor (FIG. 33A: CRF), corticosterone releasing factor receptors 1 and 2 (FIG. 33B: CRFR1, FIG. 33C: CRFR2), brain-derived neurotrophin factor (FIG. 33D: BDNF), vasopressin receptor (FIG. 33E), oxytocin receptor (FIG. 33F), glucocorticoid receptor (FIG. 33G) and mineralocorticoid receptor (FIG. 33H) were analysed in brain tissue from the hippocampus.

FIGS. 34A-34G: Effect of treatment with MRX010 on amygdala gene expression of endocrine markers. *t(11)=02.943, p=0.013. Expression of genes for endocrine markers corticosterone releasing factor receptors 1 and 2 (FIG. 34A: CRFR1, FIG. 34B: CRFR2), brain-derived neurotrophin factor (FIG. 34C: BDNF), vasopressin receptor (FIG. 34D), oxytocin receptor (FIG. 34E), glucocorticoid receptor (FIG. 34F) and mineralocorticoid receptor (FIG. 34G) were analysed in brain tissue from the amygdala.

FIGS. 35A-35F: Effect of treatment with MRX010 on PFC gene expression of endocrine markers. Expression of genes for endocrine markers corticosterone releasing factor receptors 1 and 2 (FIG. 35A: CRFR1, FIG. 35B: CRFR2), brain-derived neurotrophin factor (FIG. 35C: BDNF), oxytocin receptor (FIG. 35D), glucocorticoid receptor (FIG. 35E) and mineralocorticoid receptor (FIG. 35F) were analysed in brain tissue from the prefrontal cortex.

### DISCLOSURE OF THE INVENTION

Bacterial strains

The compositions of the invention comprise a bacterial strain of the species Enterococcus faecium. The examples demonstrate that bacteria of this species are useful for treating or preventing autism spectrum disorders and central nervous system disorders mediated by the microbiota-gut-brain axis. The mouse model experiments used in this application for the assessment of the symptoms of autism spectrum disorders are known in the art to be applicable for the assessment of the symptoms other central nervous system disorders including those listed above

The invention provides a composition comprising a bacterial strain of the species Enterococcus faecium for use in therapy, for example, for use in treating or preventing a central nervous system disorder or condition, in particular a central nervous system disorder or condition mediated by the microbiota-gut-brain axis. In certain embodiments, the compositions of the invention comprise Enterococcus faecium and do not contain any other bacterial species. In certain embodiments, the compositions of the invention comprise a single strain of Enterococcus faecium and do not contain any other bacterial strains or species.

Enterococcus faecium is a Gram-positive, alpha-hemolytic or nonhemolytic bacterium in the genus Enterococcus that often occurs in pairs (diplococci) or short chains. The type strain of Enterococcus faecium is ATCC 19434=CCUG 542=CIP 103014=CFBP 4248=DSM 20477=HAMBI 1710=JCM 5804=JCM 8727=LMG 11423=NBRC 100486=NBRC 100485=NCIMB 11508 (formerly NCDO 942)=NCTC 7171 [28]. The GenBank accession number for the 16S rRNA gene sequence of Enterococcus faecium strain LMG 11423 is AJ301830 (disclosed herein as SEQ ID NO:1). This exemplary Enterococcus faecium strain is described in [28].

Other Enterococcus faecium strains for use in the invention include: R13 [29], CFR 3003 [30], AL41 [31], DSM 10663 NCIMB 10415 [32], NCIMB 10415 E1707 [33], NM113 and NM213 [34]. In certain embodiments, the compositions of the invention comprise one of these strains, or a derivative or biotype thereof. A further example of an Enterococcus faecium for use in the invention is the DO strain. The genomic sequence of this bacterium consists of a chromosome and three plasmids. The sequence of the chromosome is disclosed herein as SEQ ID NO:3 and the sequence of the three plasmids is disclosed as SEQ ID NOs:4, 5 and 6. The genomic sequence was obtained using whole shotgun sequences and is available using GenBank accession number NC_017960.1.

The Enterococcus faecium bacterium deposited under accession number NCIMB 42487 was tested in the Examples and is also referred to herein as strain MRX010. The terms “MRX010”, “MRx0010” “Mrx010” and “Mrx0010” are used interchangeably herein. A 16S rRNA sequence for the MRX010 strain that was tested is provided in SEQ ID NO:2. Strain MRX010 was deposited with the international depositary authority NCIMB, Ltd. (Ferguson Building, Aberdeen, AB21 9YA, Scotland) by 4D Pharma Research Ltd. (Life Sciences Innovation Building, Aberdeen, AB25 2ZS, Scotland) on 16 Nov. 2015 as “Enterococcus faecium” and was assigned accession number NCIMB 42487.

Bacterial strains closely related to the strain tested in the examples are also expected to be effective for treating or preventing autism spectrum disorders and central nervous system disorders and conditions, in particular central nervous system disorders and conditions mediated by the microbiota-gut-brain axis. In certain embodiments, the bacterial strain for use in the invention has a 16s rRNA sequence that is at least 95%, 96%, 97%, 98%, 99%, 99.5% or 99.9% identical to the 16s rRNA sequence of a bacterial strain of Enterococcus faecium. Preferably, the bacterial strain for use in the invention has a 16s rRNA sequence that is at least 95%, 96%, 97%, 98%, 99%, 99.5% or 99.9% identical to SEQ ID NO:1 or 2. Preferably, the sequence identity is to SEQ ID NO:2. Preferably, the bacterial strain for use in the invention has the 16s rRNA sequence represented by SEQ ID NO:2.

Bacterial strains that are biotypes of the bacterium deposited under accession number 42487 are also expected to be effective for treating or preventing autism spectrum disorder and central nervous system disorders and conditions, in particular central nervous system disorders and conditions mediated by the microbiota-gut-brain axis. A biotype is a closely related strain that has the same or very similar physiological and biochemical characteristics.

Strains that are biotypes of the bacterium deposited under accession number NCIMB 42487 and that are suitable for use in the invention may be identified by sequencing other nucleotide sequences for the bacterium deposited under accession number NCIMB 42487. For example, substantially the whole genome may be sequenced and a biotype strain for use in the invention may have at least 95%, 96%, 97%, 98%, 99%, 99.5% or 99.9% sequence identity across at least 80% of its whole genome (e.g. across at least 85%, 90%, 95% or 99%, or across its whole genome). For example, in some embodiments, a biotype strain has at least 98% sequence identity across at least 98% of its genome or at least 99% sequence identity across 99% of its genome. Other suitable sequences for use in identifying biotype strains may include hsp60 or repetitive sequences such as BOX, ERIC, (GTG)₅, or REP or [35]. Biotype strains may have sequences with at least 95%, 96%, 97%, 98%, 99%, 99.5% or 99.9% sequence identity to the corresponding sequence of the bacterium deposited under accession number NCIMB 42487.In some embodiments, a biotype strain has a sequence with at least 95%, 96%, 97%, 98%, 99%, 99.5% or 99.9% sequence identity to the corresponding sequence of strain MRX010 deposited as NCIMB 42487 and comprises a 16S rRNA sequence that is at least 99% identical (e.g. at least 99.5% or at least 99.9% identical) to SEQ ID NO:2. In some embodiments, a biotype strain has a sequence with at least 95%, 96%, 97%, 98%, 99%, 99.5% or 99.9% sequence identity to the corresponding sequence of strain MRX010 deposited as NCIMB 42487 and has the 16S rRNA sequence of SEQ ID NO:2.

Alternatively, strains that are biotypes of the bacterium deposited under accession number NCIMB 42487 and that are suitable for use in the invention may be identified by using the accession number NCIMB 42487 deposit and restriction fragment analysis and/or PCR analysis, for example by using fluorescent amplified fragment length polymorphism (FAFLP) and repetitive DNA element (rep)-PCR fingerprinting, or protein profiling, or partial 16S or 23s rDNA sequencing. In preferred embodiments, such techniques may be used to identify other Enterococcus faecium strains.

In certain embodiments, strains that are biotypes of the bacterium deposited under accession number NCIMB 42487 and that are suitable for use in the invention are strains that provide the same pattern as the bacterium deposited under accession number NCIMB 42487 when analysed by amplified ribosomal DNA restriction analysis (ARDRA), for example when using Sau3AI restriction enzyme (for exemplary methods and guidance see, for example,[36]). Alternatively, biotype strains are identified as strains that have the same carbohydrate fermentation patterns as the bacterium deposited under accession number NCIMB 42487.

In some embodiments, the bacterial strain used in the invention is:
(i) Positive for at least one of (e.g. at least 2, 3, 4, 5, 6, 7, 8, 9, 10, 11 or all of): arginine dihydrolase, β-glucosidase, mannose fermentation, glutamic acid decarboxylase, arginine arylamidase, phenylalanine arylamidase, leucine arylamidase, pyroglutamic acid arylamidase, tyrosine arylamidase, glycine arylamidase, histidine arylamidase and serine arylamidase; and/or
(ii) Intermediate for N-acetyl-β-glucosaminidase;

preferably as determined by an assay of carbohydrate, amino acid and nitrate metabolism, and optionally an assay of alkaline phosphatase activity, more preferably as determined by Rapid ID 32A analysis (preferably using the Rapid ID 32A system from bioMérieux).

Other Enterococcus faecium strains that are useful in the compositions and methods of the invention, such as biotypes of the bacterium deposited under accession number NCIMB 42487, may be identified using any appropriate method or strategy, including the assays described in the examples. For instance, strains for use in the invention may be identified by culturing in anaerobic YCFA and/or administering the bacteria to an autism spectrum disorder mouse model and then assessing cytokine levels. In particular, bacterial strains that have similar growth patterns, metabolic type and/or surface antigens to the bacterium deposited under accession number NCIMB 42487 may be useful in the invention. A useful strain will have comparable immune modulatory activity to the NCIMB 42487 strain. In particular, a biotype strain will elicit comparable effects on the autism spectrum disorder models to the effects shown in the Examples, which may be identified by using the culturing and administration protocols described in the Examples.

A particularly preferred strain of the invention is the Enterococcus faecium strain deposited under accession number NCIMB 42487. This is the exemplary MRX010 strain tested in the examples and shown to be effective for treating disease. Therefore, the invention provides a cell, such as an isolated cell, of the Enterococcus faecium strain deposited under accession number NCIMB 42487, or a derivative thereof. The invention also provides a composition comprising a cell of the Enterococcus faecium strain deposited under accession number NCIMB 42487, or a derivative thereof. The invention also provides a biologically pure culture of the Enterococcus faecium strain deposited under accession number NCIMB 42487. The invention also provides a cell of the Enterococcus faecium strain deposited under accession number NCIMB 42487, or a derivative thereof, for use in therapy, in particular for the diseases described herein. A derivative of the strain deposited under accession number NCIMB 42487 may be a daughter strain (progeny) or a strain cultured (subcloned) from the original.

A derivative of a strain of the invention may be modified, for example at the genetic level, without ablating the biological activity. In particular, a derivative strain of the invention is therapeutically active. A derivative strain will have comparable immune modulatory activity to the original NCIMB 42487 strain. In particular, a derivative strain will elicit comparable effects on the autism spectrum disorder models to the effects shown in the Examples, which may be identified by using the culturing and administration protocols described in the Examples. A derivative of the NCIMB 42487 strain will generally be a biotype of the NCIMB 42487 strain.

References to cells of the Enterococcus faecium strain deposited under accession number NCIMB 42487 encompass any cells that have the same safety and therapeutic efficacy characteristics as the strains deposited under accession number NCIMB 42487, and such cells are encompassed by the invention. Thus, in some embodiments, reference to cells of the Enterococcus faecium strain deposited under accession number NCIMB 42487 refers only to the MRX010 strain deposited under NCIMB 42487 and does not refer to a bacterial strain that was not deposited under NCIMB 42487. In some embodiments, reference to cells of the Enterococcus faecium strain deposited under accession number NCIMB 42487 refers to cells that have the same safety and therapeutic efficacy characteristics as the strains deposited under accession number NCIMB 42487, but which are not the strain deposited under NCIMB 42487.

In certain embodiments, the bacterial strain for use in the invention has a chromosome with sequence identity to SEQ ID NO:3. In some embodiments, the bacterial strain for use in the invention has a chromosome with at least 90% sequence identity (e.g. at least 92%, 94%, 95%, 96%, 97%, 98%, 99% or 100% sequence identity) to SEQ ID NO:3 across at least 60% (e.g. across at least 65%, 70%, 75%, 80%, 85%, 95%, 96%, 97%, 98%, 99% or 100%) of SEQ ID NO:3. For example, the bacterial strain for use in the invention may have a chromosome with at least 90% sequence identity to SEQ ID NO:3 across 70% of SEQ ID NO:3, or at least 90% sequence identity to SEQ ID NO:3 across 80% of SEQ ID NO:3, or at least 90% sequence identity to SEQ ID NO:3 across 90% of SEQ ID NO:3, or at least 90% sequence identity to SEQ ID NO:3 across 100% of SEQ ID NO:3, or at least 95% sequence identity to SEQ ID NO:3 across 70% of SEQ ID NO:3, or at least 95% sequence identity to SEQ ID NO:3 across 80% of SEQ ID NO:3, or at least 95% sequence identity to SEQ ID NO:3 across 90% of SEQ ID NO:3, or at least 95% sequence identity to SEQ ID NO:3 across 100% of SEQ ID NO:3, or at least 98% sequence identity to SEQ ID NO:3 across 70% of SEQ ID NO:3, or at least 98% sequence identity to SEQ ID NO:3 across 80% of SEQ ID NO:3, or at least 98% sequence identity to SEQ ID NO:3 across 90% of SEQ ID NO:3, or at least 98% identity across 95% of SEQ ID NO:3, or at least 98% sequence identity to SEQ ID NO:3 across 100% of SEQ ID NO:3, or at least 99.5% sequence identity to SEQ ID NO:3 across 90% of SEQ ID NO:3, or at least 99.5% identity across 95% of SEQ ID NO:3, or at least 99.5% identity across 98% of SEQ ID NO:3, or at least 99.5% sequence identity to SEQ ID NO:3 across 100% of SEQ ID NO:3.

In certain embodiments, the bacterial strain for use in the invention has a chromosome with sequence identity to SEQ ID NO:3, for example as described above, and a 16S rRNA sequence with sequence identity to SEQ ID NO:1 or 2, for example as described above, preferably with a 16s rRNA sequence that is at least 99% identical to SEQ ID NO: 2, more preferably which comprises the 16S rRNA sequence of SEQ ID NO:2.

In certain embodiments, the bacterial strain for use in the invention has a chromosome with sequence identity to SEQ ID NO:3, for example as described above, and is effective for treating or preventing central nervous system disorders and conditions, in particular central nervous system disorders and conditions mediated by the microbiota-gut-brain axis.

In certain embodiments, the bacterial strain for use in the invention has a chromosome with sequence identity to SEQ ID NO:3, for example as described above, and a 16S rRNA sequence with sequence identity to SEQ ID NO: 1 or 2, for example as described above, and is effective for treating or preventing central nervous system disorders and conditions, in particular central nervous system disorders and conditions mediated by the microbiota-gut-brain axis.

In certain embodiments, the bacterial strain for use in the invention has a 16s rRNA sequence that is at least 99%, 99.5% or 99.9% identical to the 16s rRNA sequence represented by SEQ ID NO: 2 (for example, which comprises the 16S rRNA sequence of SEQ ID NO:2) and a chromosome with at least 95% sequence identity to SEQ ID NO:3 across at least 90% of SEQ ID NO:3, and which is effective for treating or preventing central nervous system disorders and conditions, in particular central nervous system disorders and conditions mediated by the microbiota-gut-brain axis.

In certain embodiments, the bacterial strain for use in the invention is a Enterococcus faecium and has a 16s rRNA sequence that is at least 99%, 99.5% or 99.9% identical to the 16s rRNA sequence represented by SEQ ID NO: 2 (for example, which comprises the 16S rRNA sequence of SEQ ID NO:2) and a chromosome with at least 98% sequence identity (e.g. at least 99% or at least 99.5% sequence identity) to SEQ ID NO:3 across at least 98% (e.g. across at least 99% or at least 99.5%) of SEQ ID NO:3, and which is effective for treating or preventing central nervous system disorders and conditions, in particular central nervous system disorders and conditions mediated by the microbiota-gut-brain axis.

In preferred embodiments, the bacterial strains in the compositions of the invention are viable and capable of partially or totally colonising the intestine.

Enterococcus faecalis and Enterococcus faecium display susceptibility to different antibiotics [25]. For example, Enterococcus faecalis is susceptible to amoxicillin, ampicillin, arbekacin and dibekacin, azlocillin, bacampicillin, carbenicillin, ceftobiprole, clarithromycin, doripenem, erythromycin, fusidic acid, gentamicin, grepafloxacin, imipenem, josamycin, meropenem, meziocillin, piperacillin, rifampin, rifaximin, rokitamycin, rosaramicin, roxithromycin, spiramycin, streptomycin, sulfamethoxazole/trimethoprim, telithromycin, ticarcillin, ticarcillin/clavulanate, tosufloxacin, trimethoprim and virginiamycin, while Enterococcus faecium demonstrates resistance to all of these antibiotics [37]. Conversely, Enterococcus faecium is susceptible to Quinopristin-dalfopristin, while Enterococcus faecalis is uniformly resistant [25][37]. In particular, Enterococcus faecium is less susceptible to β-lactam antibiotics than Enterococcus faecalis and also shows increased resistance to ampicillin and vancomycin compared to Enterococcus faecalis [25].

Enterococcus faecium is generally considered a commensal organism of the gastrointestinal tract. In contrast, Enterococcus faecalis is the most commonly encountered pathogenic Enterococcus species, likely due to important differences in its survival strategy [25]. Indeed, Enterococcus faecalis displays traits that confer a greater degree of intrinsic virulence, for example, increased cytolysin production, pheromone-responsive plasmid transfer (and accompanying production of aggregation substance), extracellular superoxide production, and unique surface proteins. Therefore, Enterococcus faecalis expresses a large number of different proteins compared to Enterococcus faecium, and these proteins are associated with increased virulence of Enterococcus faecalis. The Enterococcus faecalis cytolysin is a unique, extensively modified bacterial toxin, which is not expressed by Enterococcus faecium. Furthermore, Enterococcus faecalis expresses a number of host-parasite interaction genes (e.g. Esp), which are not expressed by Enterococcus faecium. In addition, all Enterococcus faecalis strains generate substantial extracellular superoxide, a trait not observed in Enterococcus faecium.

Unlike Enterococcus faecium, which is considered a strict fermenter, Enterococcus faecalis synthesises cytochromes using exogenous hemin and so has a growth advantage under aerobic conditions, which is associated with colonisation at inappropriate sites [25]. Furthermore, Enterococcus faecium can generate acid from L-Arabinose, while Enterococcus faecalis cannot. Conversely, Enterococcus faecalis can generate acid from L-Glyerol while Enterococcus faecium cannot. Additionally, these two bacterial species have different levels of β-galactosidase and arginine dihydrolase, and demonstrate different ability to hydrolyse starch and produce acid from melibiose, L-rhamnose, D-sorbitol and sucrose [37]. Therefore, Enterococcus faecium and Enterococcus faecalis are strikingly phenotypically distinct Enterococcus species.

In certain embodiments, the bacterial strain for use in the invention is resistant to one of more of amoxicillin, ampicillin, arbekacin and dibekacin, azlocillin, bacampicillin, carbenicillin, ceftobiprole, clarithromycin, doripenem, erythromycin, fusidic acid, gentamicin, grepafloxacin, imipenem, josamycin, meropenem, meziocillin, piperacillin, rifampin, rifaximin, rokitamycin, rosaramicin, roxithromycin, spiramycin, streptomycin, sulfamethoxazole/trimethoprim, telithromycin, ticarcillin, ticarcillin/clavulanate, tosufloxacin, trimethoprim and virginiamycin. In certain embodiments, the bacterial strain for use in the invention is susceptible to Quinopristin-dalfopristin.

In certain embodiments, the bacterial strain for use in the invention is resistant to β-lactam antibiotics. In certain embodiments, the bacterial strain for use in the invention is resistant to vancomycin. In certain embodiments, the bacterial strain for use in the invention is resistant to ampicillin.

In certain embodiments, the bacterial strain for use in the invention is a strict fermenter. In certain embodiments, the bacterial strain for use in the invention can generate acid from L-Arabinose. In certain embodiments, the bacterial strain for use in the invention cannot generate acid from glycerol.

### Therapeutic Uses

### Modulation of the Microbiota-Gut-Brain Axis

Communication between the gut and the brain (the microbiota-gut-brain axis) occurs via a bidirectional neurohumoral communication system. Recent evidence shows that the microbiota that resides in the gut can modulate brain development and produce behavioural phenotypes via the microbiota-gut-brain axis. Indeed, a number of reviews suggest a role of the microbiota-gut-brain axis in maintaining central nervous system functionality and implicate dysfunction of the microbiota-gut-brain axis in the development of central nervous system disorders and conditions [10],[13],[14],[38].

The bidirectional communication between the brain and the gut (i.e. the-gut-brain axis) includes the central nervous system, neuroendocrine and neuroimmune systems, including the hypothalamus-pituitary-adrenal (HPA) axis, sympathetic and parasympathetic arms of the autonomic nervous system (ANS), including the enteric nervous system (ENS) and the vagus nerve, and the gut microbiota.

As demonstrated in the examples, the compositions of the present invention can modulate the microbiota-gut-brain axis and reduce behavioural symptoms associated with a CNS disorder. Accordingly, the compositions of the invention may be useful for treating or preventing disorders of the central nervous system (CNS), in particular those disorders and conditions associated with dysfunction of the microbiota-gut-brain axis.

The compositions of the invention may also be useful for treating or preventing neurodevelopmental disorders and/or neuropsychiatric conditions. Neurodevelopmental diseases and neuropsychiatric conditions are often associated with the microbiota-gut-brain axis. The compositions of the invention may be useful for treating or preventing neurodevelopmental diseases and/or neuropsychiatric conditions mediated by dysfunction of the microbiota-gut-brain axis. In further preferred embodiments, the compositions of the invention are for use in treating or preventing a neurodevelopmental disorder or a neuropsychiatric condition.

In particular embodiments, the compositions of the invention may be useful for treating or preventing a disease or condition selected from the group consisting of: autism spectrum disorders (ASDs); child developmental disorder; obsessive compulsive disorder (OCD); major depressive disorder; depression; seasonal affective disorder; anxiety disorders; schizophrenia spectrum disorders; schizophrenia; bipolar disorder; psychosis; mood disorder; chronic fatigue syndrome (myalgic encephalomyelitis); stress disorder; post-traumatic stress disorder; dementia; Alzheimer's; Parkinson's disease; and/or chronic pain. In further embodiments, the compositions of the invention may be useful for treating or preventing motor neuron disease; Huntington's disease; Guillain-Barre syndrome and/or meningitis.

The compositions of the invention may be particularly useful for treating or preventing chronic disease, treating or preventing disease in patients that have not responded to other therapies (such as treatment with anti-psychotics and/or anti-depressants), and/or treating or preventing the tissue damage and symptoms associated with dysfunction of the microbiota-gut-brain axis.

In certain embodiments, the compositions of the invention modulate the CNS. In some embodiments, the compositions of the invention modulate the autonomic nervous system (ANS). In some embodiments, the compositions of the invention modulate the enteric nervous system (ENS). In some embodiments, the compositions of the invention modulate the hypothalamic, pituitary, adrenal (HPA) axis. In some embodiments, the compositions of the invention modulate the neuroendocrine pathway. In some embodiments, the compositions of the invention modulate the neuroimmune pathway. In some embodiments, the compositions of the invention modulate the CNS, the ANS, the ENS, the HPA axis and/or the neuroendocrine and neuroimmune pathways. In certain embodiments, the compositions of the invention module the levels of commensal metabolites and/or the gastrointestinal permeability of a subject.

The signalling of the microbiota-gut-brain axis is modulated by neural systems. Accordingly, in some embodiments, the compositions of the invention modulate signalling in neural systems. In certain embodiments, the compositions of the invention modulate the signalling of the central nervous system. In some embodiments, the compositions of the invention modulate signalling in sensory neurons. In other embodiments, the compositions of the invention modulate signalling in motor neurons. In some embodiments, the compositions of the invention modulate the signalling in the ANS. In some embodiments, the ANS is the parasympathetic nervous system. In preferred embodiments, the compositions of the invention modulate the signalling of the vagus nerve. In other embodiments, the ANS is the sympathetic nervous system. In other embodiments, the compositions of the invention modulate the signalling in the enteric nervous system. In certain embodiments, the signalling of ANS and ENS neurons responds directly to luminal contents of the gastrointestinal tract. In other embodiments, the signalling of ANS and ENS neurons responds indirectly to neurochemicals produced by luminal bacteria. In other embodiments, the signalling of ANS and ENS neurons responds to neurochemicals produced by luminal bacteria or enteroendocrine cells. In certain preferred embodiments, the neurons of the ENS activate vagal afferents that influence the functions of the CNS. In some embodiments, the compositions of the invention regulate the activity of enterochromaffin cells.

In certain embodiments, the compositions of the invention modulate fear conditioning in an animal model. In certain embodiments, the compositions of the invention can be used to modulate the development of fear and/or anxiety, and/or modulate the extent to which the fear and/or anxiety becomes extinct in a subject. In certain embodiments, the compositions of the invention can be used to modulate the extent of stress-induced hyperthermia in an animal model. In certain embodiments, the compositions of the invention modulate the level of stress and/or anxiety in a subject.

### Autism Spectrum Disorder (ASD)

Autism spectrum disorder is a set of heterogeneous neurodevelopmental conditions, characterised by early-onset difficulties in social interaction, communication and unusually restricted, repetitive behaviour and interests. Symptoms can be recognised from a very early age but ASD is often diagnosed in more able children starting mainstream education. Autism represents the primary type of ASD.

Historically, autism has been diagnosed on the basis of three core domains: impaired social interaction, abnormal communication, and restricted and repetitive behaviours and interests. In the International Classification of Diseases (ICD-10R, WHO 1993) and the Diagnostic and Statistical Manual (DSM-IV, American Psychiatric Association, 2000), autism comes under the umbrella term of Pervasive Developmental Disorder (PDD), with four possible diagnostic subtypes: Asperger Syndrome, Childhood Autism/Autistic Disorder, Atyptical Autism, and PDD-not otherwise specified. In DMS-5, these diagnostic subtypes are combined into a single category of autism spectrum disorder (ASD) and the previous use of three core domains of impairment has been reduced to two main areas, namely social communication and interaction, and repetitive behaviour, which include sensory integration dysfunctions.

ASD is a ‘spectrum disorder’ as it affects each person in a variety of different ways and can range from very mild to severe. The functioning of the affected individual varies substantially depending on language abilities, level of intelligence, co-morbidity, composition of symptoms and access to services. Cognitive functioning, learning, attention and sensory processing are usually impaired.

DSM-IV states that the diagnosis of autism requires the presence of at least six symptoms, including a minimum of two measures of qualitative impairment in social interaction, one symptom of qualitative impairment in communication, and one symptom of restricted and repetitive behaviour. DMS-5 redefines diagnosis of ASD into two symptom domains: (i) social interaction and social communication deficits; and (ii) restricted, repetitive patterns of behaviour, interests or activities.

Co-morbid medical conditions are highly prevalent in ASDs. Co-morbid include anxiety and depression, seizures, attention deficits, aggressive behaviours, sleep problems, gastrointestinal disorders, epilepsy, mental retardation, intellectual disabilities and feeding difficulties.

The examples demonstrate that the compositions of the invention achieve a reduction in disease incidence and disease severity in an animal model of autism spectrum disorder and so they may be useful in the treatment or prevention of autism spectrum disorders.

ASD is a central nervous system disorder that is partially triggered by environmental factors. Therefore, dysfunction of the microbiota-gut-brain axis may be responsible for development and persistence of ASDs. Accordingly, in preferred embodiments, the composition of the invention are for use in treating or preventing autism spectrum disorders. In some embodiments, the compositions of the invention are for use in treating or preventing autism. In some embodiments, the autism is Pervasive Developmental Disorder (PDD). In another embodiment, the PDD is Asperger Syndrome, Childhood Autism/Autistic Disorder, Atyptical Autism and/or PDD-not otherwise specified. Accordingly, in some embodiments, the compositions of the invention are for use in treating or preventing autism spectrum disorders, autism, pervasive developmental disorder; Asperger Syndrome; Childhood Autism/Autistic Disorder, Atypical Autism and/or PDD-not otherwise specified.

The compositions of the invention may be useful for modulating the microbiota-gut-brain axis of a subj ect. Accordingly, in preferred embodiments the compositions of the invention are for use in preventing an ASD in a patient that has been identified as at risk of an ASD, or that has been diagnosed with an ASD at a prenatal or an early developmental stage; in childhood and/or in adulthood. The compositions of the invention may be useful for preventing the development of ASDs.

The compositions of the invention may be useful for managing or alleviating ASDs. Treatment or prevention of ASDs may refer to, for example, an alleviation of the severity of symptoms or a reduction in the frequency of exacerbations or the range of triggers that are a problem for the patient.

In preferred embodiments, the compositions of the invention prevent, reduce or alleviate at least one core symptom of ASDs.

In some embodiments, the compositions of the invention prevent, reduce or alleviate at least one of the two symptom domains of ASD classified in the DMS-5. In some embodiments, the compositions of the invention prevent, reduce or alleviate social interaction and/or social communication deficits. In some embodiments, the compositions of the invention prevent, reduce or alleviate restrictive, repetitive patterns of behaviour, interests or activities. In some embodiments, the compositions of the invention prevent, reduce or alleviate social interaction, social communication deficits and/or restrictive, repetitive patterns of behaviour, interests or activities.

In some embodiments, the compositions of the invention prevent, reduce or alleviate repetitive behaviour, stereotyped behaviour, compulsive behaviour, routine behaviour, sameness behaviour and restricted behaviour. In some embodiments, the compositions of the invention improve social awareness, social information processing, capacity for social communication, social anxiety/avoidance, and autistic preoccupations and traits in a subject with ASDs.

In some embodiments, the compositions of the invention prevent, reduce or alleviate additional symptoms associated with the core symptoms of ASDs. In some embodiments, the compositions of the invention prevent, reduce or alleviate irritability (including aggression, deliberate self-injury and temper tantrums), agitation, crying, lethargy, social withdrawal, stereotypic behaviour, hyperactivity, non-compliance, inappropriate speech, anxiety, depression, and/or over or under-controlled behaviour in a subject with ASDs. In some embodiments, the compositions of the invention improve cognitive functioning, learning, attention and/or sensory processing in a subject with ASD.

In other embodiments, the compositions of the invention improve secondary outcome measures in a subject with ASDs. In some embodiments, the secondary outcome measures include additional symptom and/or functional rating scales, behavioural scales and miscellaneous measures of interest.

In some embodiments, the compositions of the invention cause in a positive change in the diagnostic and/or symptomatic scale for the assessment of core symptoms of a subject with ASDs. In some embodiments, the diagnostic and/or symptomatic scale is the Autism Diagnostic Interview—Revised (ASI-R). In some embodiments, the diagnostic or symptomatic scale is the Autism Diagnostic Observation Schedule-Generic (ADOS-G) now ADOS-2. In other embodiments, the diagnostic or symptomatic scale is the Autism Diagnostic Interview Revised (ADI-R). In other embodiments, the diagnostic or symptomatic scale is the Diagnostic Interview for Social and Communication Disorders (DISCO). In yet other embodiments, the diagnostic or symptomatic scale is the Childhood Autism Rating Scale (CARS and CARS2).

In some embodiments, the compositions of the invention cause a positive change in generic measures of the efficacy endpoints of ASDs. In certain embodiments, the generic measures include, but are not limited to the Aberrant Behaviour Checklist (ABC), the Child Behaviour Checklist (CBCL), the Vineland-II Adaptive Behaviour Scales (VABS), the Social Responsiveness Scale (SRS), and/or the Repetitive Behaviour Scale—Revised (RBS-R).

In some embodiments, the compositions of the invention improve the Clinical Global Impression—Global Improvement (CGI-I) scale for assessing psychiatric and neurological disorders. In some embodiments, the compositions of the invention display a positive effect on global functioning of the subject with ASDs.

Additional scales would be known to a person skilled in the art. In some embodiments, the compositions of the invention would improve the outcome of diagnostic and/or symptomatic scales known to a person skilled in the art.

In certain embodiments, the compositions of the invention prevent, reduce or alleviate the incidence of comorbidities of ASDs. In some embodiments, the compositions of the invention prevent, reduce or alleviate the incidence of anxiety and depression, seizures, attention deficits, aggressive behaviours, sleep problems, gastrointestinal disorders (including irritable bowel syndrome (IBS)), epilepsy, mental retardation, intellectual disabilities and/or feeding difficulties. In certain embodiments, the compositions of the invention prevent, reduce or alleviate gastrointestinal comorbidities, such as abdominal pain, diarrhoea and flatulence.

In some embodiments, the compositions of the invention prevent, reduce or alleviate the symptoms of certain psychiatric and behavioural disorders that may present clinically with similarities to autism. Accordingly, in some embodiments, the compositions of the invention, prevent, reduce or alleviate attention deficit disorder (ADHD); affective/anxiety disorders; attachment disorders; oppositional defiant disorder (ODD); obsessive compulsive disorder (OCD) and/or psychoses including schizophrenia (cognitive impairment).

In some embodiments, the compositions of the invention are particularly effective at preventing, reducing or alleviating ASDs when used in combination with another therapy for treating ASDs. Such therapies include anti-psychotic, anti-anxiety and anti-depressant drugs. Such drugs include risperidone (Risperdal®); olanzapine (Zyprexa®); fluoxetine (Prozac®); sertraline (Zoloft®); fluvoxamine (Luvox®); clomipramine (Anafranil®); haloperidol (Haldol®); thioridazine; fluphenazine; chlorpromazine; ziprasidone (Geogon®); carbamazepine (Tegretol®); lamotrigine (Lamictal®); topiramate (Topomax®); valproic acid (Depakote®); methylphenidate (Ritalin®); diazepam (Valium®) and lorazepam (Ativan®).

### Obsessive Compulsive Disorder (OCD)

OCD is a heterogeneous, chronic and disabling disorder belonging to the anxiety disorders. According to the DSM-IV definition, the essential features of OCD are recurrent obsessions and/or compulsions (criterion A) that are severe and time consuming (more than one hour a day) or cause marked distress or significantly interfere with the subject's normal routine, occupational functioning, usual social activities or relationships (criterion C). As some point during the course of the disorder, the person has recognised that the obsessions or compulsions are excessive or unreasonable (criterion B).

Obsessions are defined as recurrent and persistent thoughts, impulses or images that are experienced as intrusive and inappropriate and cause marked anxiety or distress. The thoughts, impulses or images are not simply excessive worries about real-life problems, they are recognised by the patient as a product of his own mind (e.g. fear for contamination, symmetry obsession). The person attempts to ignore, suppress or neutralise the obsessions with some other thoughts or actions.

Compulsions are defined as repetitive behaviours (e.g. hand washing, ordering, hoarding, checking) or mental acts (e.g. praying, counting, repeating words silently) that the person feels driven to perform in response to an obsession or according to rules that must be applied rigidly.

OCD is often associated with co-morbidity rates of other psychiatric diseases including major depressive disorder, other anxiety disorders (generalised anxiety disorder, social anxiety disorder, panic disorder), substance abuse and eating disorders (anorexia and bulimia).

OCD is a psychiatric disorder that may develop or persist due to dysfunction of the microbiota-gut-brain axis. Accordingly, in preferred embodiments, the compositions of the invention are for use in treating or preventing OCD in a subject.

In certain embodiments, the compositions of the invention prevent, reduce or alleviate the essential symptomatic features of OCD. In certain embodiments, the compositions of the invention prevent, reduce or alleviate recurrent obsessions and/or compulsions in a subject. In certain embodiments, the obsessions are recurrent or persistent thoughts, impulses or images that are experiences as intrusive and inappropriate and cause marked anxiety or distress. In certain embodiments, the compulsions are repetitive behaviours that the subject feels driven to perform in response to an obsession or according to rules that must be applied rigidly.

In certain embodiments, the compositions of the invention improve symptoms of OCD in a subject accordingly to the Y-BOCS and/or the NIMH-OC diagnostic and/or symptomatic scales. In some embodiments, the Y-BOCS scale is used to monitor improvement of primary endpoints. In some embodiments, the NIMH-OC scale is used to monitor improvement of secondary parameters.

In some embodiments, the compositions of the invention improve the Clinical Global Impression—Global Improvement (CGI-I) scale for assessing psychiatric and neurological disorders. In some embodiments, the compositions of the invention display a positive effect on global social functioning (relationships, work, etc.) of the subject with ASDs. In some embodiments, the global scale is the Sheehan disability scale.

In preferred embodiments, the compositions of the invention prevent, reduce or alleviate at least one comorbidity of OCD. The comorbidities of OCD include major depressive disorder, other anxiety disorders (generalised anxiety disorder, social anxiety disorder, panic disorder), substance abuse and eating disorders (anorexia and bulimia) Gilles de la Tourette syndrome, ADHD (Attention-Deficit/Hyperactivity Disorder) and developmental disorders.

In some embodiments, the compositions of the invention are particularly effective at preventing, reducing or alleviating OCD when used in combination with another therapy for treating OCD. Such therapies include serotonin and dopamine reuptake inhibitors; clomipramine and anti-psychotics.

### Major Depressive Disorder (MDD)

MDD is associated with substantial psychosocial dysfunction and high individual mental strain as well as with excess morbidity and mortality (the risk of suicide is considerable). The term major depressive disorder encompasses clinical depression, major depression, unipolar depression, unipolar disorder, recurrent depression and simply depression. The term major depressive disorder covers mood disorders; dysthymia; chronic depression; seasonal affective disorder and borderline personality disorder.

According to the DMS-5 criteria, MDD symptoms include a depressed mood, or loss of interest or pleasure in daily activities for more than two weeks; and impaired social, occupational and educational function. Specific symptoms, at least five of the following nine, present nearly every day: depressed mood or irritable most of the day; decreased interest or pleasure in most activities, most of each day; significant weight change or change in appetite; change in sleep (insomnia or hypersomnia); change in activity (psychomotor agitation or retardation); fatigue or loss of energy; guilt or worthlessness (feelings of worthlessness or excessive or inappropriate guilt); reduced concentration (diminished ability to think or concentrate, or more indecisiveness; and suicidality (thoughts of death or suicide, or subject has a suicide plan). In addition, MDD is associated with anxiety symptoms including irrational worry; preoccupation with unpleasant worries; trouble relaxing and/or feeling tense. MDD episodes can be mild, moderate or severe.

MDD episodes are often associated with comorbidity with other psychiatric disorders or with somatic disorders like Parkinson's disease, Alzheimer's disease, cerebrovascular disorders, cancer and chronic pain syndromes. MDD is frequently associated with a wide spectrum of other mental disorders as comorbidities including generalised anxiety disorder; anxiety disorder; substance use disorders; post-traumatic stress disorder (PTSD); personality disorders; pain; stress; irritable bowel syndrome; insomnia; headaches and interpersonal problems.

Major depressive disorder is a psychiatric disorder that may develop or persist due to dysfunction of the microbiota-gut-brain axis. Accordingly, in preferred embodiments, the compositions of the invention are for use in treating or preventing MDD in a subject.

In certain embodiments, the compositions of the invention are for use in treating or preventing acute major depressive episodes and/or the prevention of new episodes (recurrence prevention). In certain embodiments, the compositions of the invention prevent, reduce or alleviate the occurrence of mild, moderate or severe MDD episodes.

In certain embodiments, the compositions of the invention prevent, reduce or alleviate one or more of the symptoms of MDD as classified by the DMS-5 criteria listed herein. In a preferred embodiment, the compositions of the invention prevent, reduce or alleviate a depressed mood in a subject. In a preferred embodiment, the compositions of the invention prevent, reduce or alleviate a decreased interest or pleasure in most activities in a subject. In some embodiments, the compositions of the invention reduce the occurrence of symptoms of MDD within a 2-week period.

In some embodiments, the compositions of the invention improve the symptoms of MDD according to a symptomatic or diagnostic scale. Such scales for assessing symptomatic improvement include the Hamilton Rating Scale of Depression (HAMD) and the Montgomery Asberg Depression Rating Scale. In addition, the Zung Self-Rating Depression Scale (SDS) and Zung Self-Rating Anxiety Scale (SAS) are also suitable symptomatic improvement scales.

In some embodiments, the compositions of the invention improve the Clinical Global Impression—Global Improvement (CGI-I) scale for assessing psychiatric and neurological disorders. In some embodiments, the compositions of the invention display a positive effect on global social and occupational functioning of the subject with MDD.

In certain embodiments, the compositions of the invention are for use in treating or preventing treatment resistant MDD.

In preferred embodiments, the compositions of the invention prevent, reduce or alleviate at least one comorbidity of MDD. The comorbidities of MDD include generalised anxiety disorder; anxiety disorder; substance use disorders; post-traumatic stress disorder (PTSD); personality disorders; pain; stress; IBS; insomnia; headaches and interpersonal problems.

In some embodiments, the compositions of the invention are particularly effective at preventing, reducing or alleviating MDD when used in combination with another therapy for treating MDD. Such therapies include antidepressants, augmentation strategies (e.g. combination therapy, lithium and other mood stabilizers, thyroid hormones and atypical antipsychotics) or even second generation antipsychotics.

### Anxiety Disorders

Anxiety disorders are a group of mental disorders characterised by feelings of anxiety and fear. There are a number of anxiety disorders including generalised anxiety disorder (GAD); specific phobia; social anxiety disorder; separation anxiety disorder; agroraphobia; panic disorder and selective mutism.

GAD is diagnosed according to DMS-5 in six criterion. The first criterion is too much anxiety or worry over more than six months wherein the anxiety or worry is present most of the time in regards to many activities. The second criterion is that the subject is unable to manage the symptoms of the first criterion. The third criterion is that at least three (one in children) of the following occurs: restlessness; tires easily; problems concentrating; irritability; muscle tension and problems with sleep. The final three criterion are that the symptoms results in significant social, occupational and functional impairment; the symptoms are not due to medications, drugs, or other physical health problems; and the symptoms do not fit better with another psychiatric problem such as panic disorder. All other anxiety disorders may be considered as differential diagnoses of GAD.

GAD is frequently associated with a wide spectrum of other mental disorders as comorbidities including depression; substance use disorders; stress; IBS; insomnia; headaches; pain; cardiac events; interpersonal problems and ADHD.

Anxiety disorders are psychiatric disorders that may develop or persist due to dysfunction of the microbiota-gut-brain axis. Accordingly, in preferred embodiments, the compositions of the invention are for use in treating or preventing anxiety disorders in a subject. In certain embodiments, the anxiety disorder is generalised anxiety disorder (GAD); specific phobia; social anxiety disorder; separation anxiety disorder; agoraphobia; panic disorder and selective mutism.

In certain embodiments, the compositions of the invention prevent, reduce or alleviate one or more of the symptoms of GAD in a subject as classified by the DMS-5 criteria listed herein. According to DMS-5, the same symptoms are associated with other anxiety disorders. Therefore, in certain embodiments, the compositions of the invention prevent, reduce or alleviate one or more of the symptoms of anxiety disorders in a subject. In preferred embodiments, the compositions of the invention prevent, reduce or alleviate the anxiety or worry of the subject. In certain embodiments, the compositions of the invention reduce the occurrence of symptoms within a six month period. In certain embodiments, the composition of the invention prevents, reduces or alleviates restlessness; fatigue; loss of concentration; irritability; muscle tension; and/or problems with sleep. In some embodiments, the compositions of the invention prevent, reduce or alleviate social, occupational and functional impairment associated with anxiety disorders.

In some embodiments, the compositions of the invention improve the symptoms of anxiety disorders according to a symptomatic or diagnostic scale. In certain embodiments, the scale for assessing symptomatic improvement includes the Hamilton Anxiety Rating Scale (HAM-A). In some embodiments, the HAM-A total scale is used to assess primary endpoint. In other embodiments, the HAM-A psychic anxiety factor may be useful as a secondary endpoint.

In some embodiments, the compositions of the invention improve the Clinical Global Impression—Global Improvement (CGI-I) scale for assessing psychiatric and neurological disorders. In some embodiments, the compositions of the invention display a positive effect on global social, occupational and functional impairment of the subject with anxiety disorder. In some embodiments, the global scale is the Sheehan disability scale.

In preferred embodiments, the compositions of the invention prevent, reduce or alleviate at least one comorbidity of GAD and anxiety disorders. The comorbidities of GAD include depression; substance use disorders; stress; IBS; insomnia; headaches; pain; cardiac events; interpersonal problems and ADHD.

In some embodiments, the compositions of the invention are particularly effective at preventing, reducing or alleviating anxiety disorders when used in combination with another therapy for treating anxiety disorders. Such therapies include selective serotonin reuptake inhibitors (venlafaxine, duloxetine, escitalopram and paroxetine); benzodiazepines (alprazolam, lorazepam and clonazepam); pregabalin (Lyrica®) and gabapentin (Neurontin®); serotonin receptor partial agonists (buspirone and tandospirone); atypical serotonergic antidepressants (such as imipramine and clomipramine); monoamine oxidase inhibitors (MAOIs) (such as moclobemide and phenelzine); hydroxyzine; propranolol; clonidine; guanfacine and prazosin.

### Post-Traumatic Stress Disorder (PTSD)

PTSD is a severe and disabling disorder, an essential feature of which is the inclusion of a traumatic event as a precipitating factor of this disorder.

The symptoms of PTSD are grouped into four main clusters according to the DMS-V criteria: (i) intrusion: examples include nightmares, unwanted thoughts of the traumatic events, flashbacks, and reacting to traumatic reminders with emotional distress or physiological reactivity; (ii) avoidance: examples include avoiding triggers for traumatic memories including places, conversations, or other reminders; (iii) negative alterations in cognitions and mood: examples include distorted blame of self or others for the traumatic event, negative beliefs about oneself or the world, persistent negative emotions (e.g., fear, guilt, shame), feeling alienated, and constricted affect (e.g., inability to experience positive emotions); (iv) alterations in arousal and reactivity: examples include angry, reckless, or self-destructive behaviour, sleep problems, concentration problems, increased startle response, and hypervigilance.

Symptoms that resolve within 4 weeks of the traumatic event meet the criteria for an Acute Stress Disorder. The DSM distinguishes between acute (duration of symptoms for less than three months) and chronic PTSD (duration of symptoms longer than 3 months). If the symptoms begin more than 6 months after the stressor, the disorder is defined as delayed onset PTSD.

PTSD carries high comorbidities with major depressive disorder and substance use disorders.

PTSD is a psychiatric disorder that may develop or persist due to dysfunction of the microbiota-gut-brain axis. Accordingly, in preferred embodiments, the compositions of the invention are for use in treating or preventing PTSD in a subject. According to a similar pathogenesis, in certain embodiments, the compositions of the invention are for use in treating or preventing stress disorders. In certain embodiments, the compositions of the invention treat acute stress disorder. In some embodiments, the compositions of the invention treat acute and/or chronic PTSD. In some embodiments, the compositions of the invention treat delayed onset PTSD.

In certain embodiments, the compositions of the invention prevent, reduce or alleviate one or more of the symptoms of PTSD (or stress disorder) in a subject as classified by the DMS-5 criteria listed herein. In preferred embodiments, the compositions of the invention prevent, reduce or alleviate intrusive thoughts in a subject with PTSD. In preferred embodiments, the compositions of the invention prevent, reduce or alleviate avoidance behaviour in a subject with PTSD. In preferred embodiments, the compositions of the invention prevent, reduce or alleviate negative alterations in cognitions and mood in a subject with PTSD. In preferred embodiments, the compositions of the invention prevent alterations in arousal and reactivity in a subject with PTSD.

In some embodiments, the compositions of the invention improve the symptoms of PTSD and stress disorders according to a symptomatic or diagnostic scale. In certain embodiments, the scale for assessing symptomatic improvement is the Clinical-Administered PTSD (CAPS) scale.

In some embodiments, the compositions of the invention improve the Clinical Global Impression—Global Improvement (CGI-I) scale for assessing psychiatric and neurological disorders. In some embodiments, the compositions of the invention display a positive effect on global social, occupational and functional impairment of the subject with PTSD and stress disorders. In some embodiments, the global scale is the Sheehan disability scale.

In preferred embodiments, the compositions of the invention prevent, reduce or alleviate at least one comorbidity of PTSD and stress disorders. The comorbidities of PTSD and stress disorders include MDD, substance use disorders; stress and anxiety.

In some embodiments, the compositions of the invention are particularly effective at preventing, reducing or alleviating PTSD and stress disorders when used in combination with another therapy for treating PTSD and stress disorders. Such therapies include serotoninergic agents, tricyclic antidepressants, mood stabilisers, adrenergic inhibiting agents, antipsychotics, benzodiazepines, sertraline (Zoloft®), fluoxetine (Prozac®) and/or paroxetine (Paxil®).

### Schizophrenia Spectrum and Psychotic Disorders

These diseases affect a subject's ability to think clearly, make good judgements, respond emotionally, communicate effectively, understand reality, and behave appropriately. Psychotic diseases include schizophrenia (symptoms listed below); schizoaffective disorder (the subject has symptoms of both schizophrenia and a mood disorder, such as depression or bipolar disorder); schizophreniform disorder (displays the symptoms of schizophrenia, but the symptoms last for a shorter time: between 1 and 6 months); brief psychotic disorder (subjects display a sudden, short period of psychotic behaviour, often in response to a very stressful event, such as a death in the family—recovery is usually less than a month); delusional disorder (delusions last for at least 1 month); shared psychotic disorder; substance-induced psychotic disorder; psychotic disorder due to another medical condition; paraphrenia (displaying symptoms similar to schizophrenia and starting late in life, when people are elderly). The most well-known psychotic disorder is schizophrenia and the majority of psychotic disorders display similar symptoms to schizophrenia.

Schizophrenia is a severe psychiatric disease with a heterogeneous course and symptom profile. Schizophrenia presents clinically with so-called positive and negative symptoms. The positive symptoms include delusions, hallucinations, disorganised speech, and disorganised or catatonic behaviours. Negative symptoms include affective flattening, restriction in the fluency and productivity of thought and speech and in the initiation of goal directed behaviour. The positive symptoms appear to reflect an excess or distortion of normal functions, whereas negative symptoms appear to reflect a diminution or loss of normal function. In addition, cognitive deficits (defects of working memory, information processing, attention/vigilance, learning, reasoning and social cognition) are common. Cognitive deficits generally show poor improvement with current antipsychotic treatment. Schizophrenic patients also suffer from mood symptoms. Besides these predominant symptoms, schizophrenia is associated with a comorbidity with other psychiatric symptoms such as manic and depressive symptoms, anxiety or obsessive-compulsive symptoms, substance abuse and dependence, and personality disorder.

According to the DMS-5, for the diagnosis of schizophrenia, a subject must have at least two of the following symptoms: delusions; hallucinations; disorganised speech; disorganised or catatonic behaviour and negative symptoms. At least one of the symptoms must be the presence of delusions, hallucinations or disorganised speech. Continuous signs of disturbance must persist for at least 6 months, during which the subject must experience at least 1 month of active symptoms, with social or occupational deterioration problems occurring over a significant amount of time.

Schizophrenia spectrum and psychotic disorders are psychiatric disorders that may develop or persist due to dysfunction of the microbiota-gut-brain axis. Therefore, in preferred embodiments, the compositions of the invention are for use in treating or preventing schizophrenia spectrum and/or psychotic disorders in a subject. In certain embodiments, the schizophrenia spectrum and psychotic disorder is selected from schizophrenia; schizoaffective disorder; schizophreniform disorder; brief psychotic disorder; delusional disorder; shared psychotic disorder; substance-induced psychotic disorder; psychotic disorder due to another medical condition and paraphrenia. In preferred embodiments, the compositions of the invention are for use in treating or preventing schizophrenia. In certain embodiments, the schizophrenia is selected from paranoid, disorganised, catatonic, undifferentiated and residual schizophrenia.

In certain embodiments, the compositions of the invention prevent, reduce or alleviate one or more of the symptoms of schizophrenia in a subject as classified by the DMS-5 criteria listed herein. These embodiments apply to the prevention, reduction or alleviation of symptoms of other schizophrenia spectrum and psychotic disorders. In certain embodiments, the compositions of the invention prevent, reduce or alleviate negative symptoms of schizophrenia. In certain embodiments, the compositions of the invention prevent, reduce or alleviate positive symptoms of schizophrenia. In certain embodiments, the compositions of the invention prevent, reduce or alleviate negative and positive symptoms of schizophrenia. In preferred embodiments, the compositions of the invention prevent, reduce or alleviate delusions, hallucinations, disorganised speech, and disorganised or catatonic behaviours in a subject with schizophrenia. In preferred embodiments, the compositions of the invention prevent, reduce or alleviate affective flattening, restriction in the fluency and productivity of thought and speech and in the initiation of goal directed behaviour in a subject with schizophrenia. In preferred embodiments, the compositions of the invention prevent, reduce or alleviate the cognitive defects and/or mood disorders in a subject with schizophrenia.

In certain embodiments, the compositions of the invention reduce the occurrence of positive and/or negative symptoms of schizophrenia in a subject within a 6 month period. In certain embodiments, the compositions of the invention improve social and/or occupational functionality in a subject with schizophrenia spectrum or psychotic disorder.

In some embodiments, the compositions of the invention improve the symptoms of schizophrenia spectrum or psychotic disorders according to a symptomatic or diagnostic scale. In certain embodiments, the scale for assessing symptomatic improvement is the Positive and Negative Symptom Scale (PANSS) and Brief Psychiatric Rating Scale (BPRS). In certain embodiments, the Scale for Assessment of Negative Symptoms (SANS) is used.

In some embodiments, the compositions of the invention improve the Clinical Global Impression—Global Improvement (CGI-I) scale for assessing psychiatric and neurological disorders. In some embodiments, the compositions of the invention display a positive effect on global social and occupational impairment of the subject with schizophrenia spectrum or psychotic disorders.

In preferred embodiments, the compositions of the invention prevent, reduce or alleviate at least one comorbidity of schizophrenia spectrum or psychotic disorder. In certain embodiments, the comorbidity is as manic and depressive symptoms, anxiety or obsessive-compulsive symptoms, substance abuse and dependence, and personality disorder.

In certain embodiments, the compositions of the invention are for use in treating or preventing treatment resistant of refractory schizophrenia.

In some embodiments, the compositions of the invention are particularly effective at preventing, reducing or alleviating schizophrenia spectrum or psychotic disorders when used in combination with another therapy for treating PTSD and stress disorders. In certain embodiments, such therapies include first generation antipsychotics including chlorpromazine, fluphenazine, haloperidol and/or perphenazine. In certain embodiments, such therapies include second generation therapies including aripiprazole (Abilify®); asenapine (Saphris®); brexpiprazole (Rexulti®); cariprazine (Vraylar®); clozapine (Clozaril®); iloperidone (Fanapt®); lurasidone (Latuda®); olanzapine (Zyprexa®); paliperidone (Invega); quetiapine (Seroquel®); risperidone (Risperdal®); ziprasidone (Geodon®).

### Bipolar Disorder

Bipolar disorder in general is a chronic disease. Mania is the cardinal symptom of bipolar disorder. There are several types of bipolar disorder based upon the specific duration and pattern of manic and depressive episodes. In DMS-5, a distinction is made between bipolar I disorder, bipolar II disorder, cyclothymic disorder, rapid-cycling bipolar disorder and bipolar disorder NOS.

According to the DSM, mania is a distinct period of abnormally and persistently elevated, expansive, or irritable mood. The episode must last a week, and the mood must have at least three of the following symptoms: high self-esteem; reduced need for sleep; increase rate of speech; rapid jumping of ideas; easily distracted; an increased interest in goals or activities; psychomotor agitation; increased pursuit of activities with a high risk of danger.

Bipolar I disorder involves one or more manic or mixed (mania and depression) episodes and at least one major depressive episode (see above for symptoms of MDD episodes). Bipolar II disorder has one or more major depressive episodes accompanied by at least one hypomanic episode. There are no manic or mixed episodes. Hypomania is a lesser form of mania. The symptoms are responsible for significant social, occupational and functional impairments. Cyclothymia is characterized by changing low-level depression along with periods of hypomania. The symptoms must be present for at least two years in adults or one year in children before a diagnosis can be made. Symptom free periods in adults and children last no longer than two months or one month, respectively. Rapid cycling bipolar disorder is a severe form of bipolar disorder. It occurs when a person has at least four episodes of major depression, mania, hypomania, or mixed states within a year. Not-otherwise specified (NOS) bipolar disorder classified bipolar symptoms that do not clearly fit into other types. NOS is diagnosed when multiple bipolar symptoms are present but not enough to meet the label for any of the other subtypes.

Bipolar disorder is associated with the following comorbidities: ADHD; anxiety disorders; substance disorders; obesity and metabolic syndrome.

Bipolar disorder is a psychiatric disorder that may develop or persist due to dysfunction of the microbiota-gut-brain axis. Therefore, in preferred embodiments, the compositions of the invention are for use in treating or preventing bipolar disorder in a subject. In certain embodiments, the bipolar disorder is bipolar I disorder. In certain embodiments, the bipolar disorder is bipolar II disorder. In certain embodiments, the bipolar disorder is cyclothymic disorder. In certain embodiments, the bipolar disorder is rapid-cycling bipolar disorder. In certain embodiments, the bipolar disorder is bipolar disorder NOS.

In preferred embodiments, the compositions of the invention prevent, reduce or alleviate one or more of the symptoms of bipolar disorder in a subject. In certain embodiments, the compositions of the invention prevent, reduce or alleviate the occurrence of manic episodes in a subject. In certain embodiments, the compositions of the invention prevent, reduce or alleviate the occurrence of an abnormally and persistently elevated, expansive, or irritable mood. In certain embodiments, the compositions of the invention prevent, reduce or alleviate one or more of the following symptoms: high self-esteem; reduced need for sleep; increase rate of speech; rapid jumping of ideas; easily distracted; an increased interest in goals or activities; psychomotor agitation; increased pursuit of activities with a high risk of danger. In certain embodiments, the compositions of the invention prevent, reduce or alleviate the occurrence of one or more manic or mixed episodes in a subject. In certain embodiments, the compositions of the invention reduce the occurrence of at least one major depressive episode in a subject. In certain embodiments, the compositions of the invention prevent, reduce or alleviate the occurrence of at least one major depressive episode accompanied by at least one hypomanic episode.

In preferred embodiments, the compositions of the invention treat the acute phase of bipolar disorder and/or prevent the occurrence of further episodes. In certain embodiments, the compositions of the invention treat the acute phase of manic/depressive episodes in a subject with bipolar disorder and prevent occurrence of further manic/depressive episodes.

In some embodiments, the compositions of the invention improve the symptoms of bipolar disorder according to a symptomatic or diagnostic scale. In certain embodiments, the scale for assessing symptomatic improvement of manic episodes is the Manic State Rating Scale and the Young Mania Rating Scale. In certain embodiments, the scale is the Bech-Rafaelsen Mania Scale (BRMAS). In certain embodiments, scales for assessing symptomatic improvement of the switch from manic to depressive episodes include the Hamilton Depression Rating Scale, the Montgomery-Asberg Rating Scale, and the Bech-Rafaelsen Depression Scale.

In some embodiments, the compositions of the invention improve the Clinical Global Impression—Global Improvement (CGI-I) scale for assessing psychiatric and neurological disorders. In some embodiments, the compositions of the invention display a positive effect on global social, occupational and functional impairments of the subject with bipolar disorder.

In preferred embodiments, the compositions of the invention prevent, reduce or alleviate at least one comorbidity of bipolar disorder. In certain embodiments, the comorbidity is selected from ADHD, anxiety disorders, substance disorder, obesity and metabolic syndrome.

In certain embodiments, the compositions of the invention are for use in treating or preventing manic-depressive illness and bipolar disorder unresponsive to lithium and divalproex.

In some embodiments, the compositions of the invention are particularly effective at preventing, reducing or alleviating bipolar disorder when used in combination with another therapy for treating bipolar disorder. In certain embodiments, such therapies include lithium carbonate, anticonvulsant drugs (including valproate, divalproex, carbamazepine and lamotrigine) and antipsychotic drugs (including aripiprazole, olanzapine, quetiapine and risperidone).

### Neurocognitive Disorders and Alzheimer's Disease

In DSM-5, the term dementia was replaced with the terms major neurocognitive disorder and mild neurocognitive disorder. Neurocognitive disorder is a heterogeneous class of psychiatric diseases. The most common neurocognitive disorder is Alzheimer's disease, followed by vascular dementias or mixed forms of the two. Other forms of neurodegenerative disorders (e.g. Lewy body disease, frontotemporal dementia, Parkinson's dementia, Creutzfeldt-Jakob disease, Huntington's disease, and Wernicke-Korsakoff syndrome) are accompanied by dementia.

The symptomatic criteria for dementia under DSM-5 are evidence of significant cognitive decline from a previous level of performance in one or more cognitive domains selected from: learning and memory; language; executive function; complex attention; perceptual-motor and social cognition. The cognitive deficits must interfere with independence in everyday activities. In addition, the cognitive deficits do not occur exclusively in the context of a delirium and are not better explained by another mental disorder (for example MDD or schizophrenia).

In addition to the primary symptom, subjects with neurocognitive disorders display behavioural and psychiatric symptoms including agitation, aggression, depression, anxiety, apathy, psychosis and sleep-wake cycle disturbances.

Neurocognitive disorders are psychiatric disorders that may develop or persist due to dysfunction of the microbiota-gut-brain axis. Therefore, in preferred embodiments, the compositions of the invention are for use in treating or preventing neurocognitive disorders in a subject. In preferred embodiments, the neurocognitive disorder is Alzheimer's disease. In other embodiments, the neurocognitive disorder is selected from vascular dementias; mixed form Alzheimer's disease and vascular dementia; Lewy body disease; frontotemporal dementia; Parkinson's dementia; Creutzfeldt-Jakob disease; Huntington's disease; and Wernicke-Korsakoff syndrome.

In preferred embodiments, the compositions of the invention prevent, reduce or alleviate one or more of the symptoms of neurocognitive disorders in a subject. In certain embodiments, the compositions of the invention prevent, reduce or alleviate the occurrence of cognitive decline in a subject. In certain embodiments, the compositions of the invention improve the level of performance of a subject with neurocognitive disorders in one or more cognitive domains selected from: learning and memory; language; executive function; complex attention; perceptual-motor and social cognition. In some embodiments, the compositions of the invention prevent, reduce or alleviate the occurrence of one or more behavioural and psychiatric symptoms associated with neurocognitive disorders selected from agitation, aggression, depression, anxiety, apathy, psychosis and sleep-wake cycle disturbances.

In certain embodiments, the compositions of the invention prevent, reduce or alleviate symptomatic disease by intervention in suspected pathogenic mechanisms at a preclinical stage. In certain embodiments, the compositions of the invention improve disease modification, with slowing or arrest of symptom progression. In some embodiments, the slowing or arrest of symptom progression correlates with evidence in delaying the underlying neuropathological process. In preferred embodiments, the compositions of the invention improve symptoms of neurocognitive disorders comprising enhanced cognitive and functional improvement. In preferred embodiments, the compositions of the invention improve the behavioural and psychiatric symptoms of dementia (BPSD). In preferred embodiments, the compositions of the invention improve the ability of a subject with neurocognitive disorder to undertake everyday activities.

In preferred embodiments, the compositions of the invention improve both cognition and functioning in a subject with Alzheimer's disease. In some embodiments, the composition of the invention improve the cognitive endpoint in a subject with Alzheimer's disease. In some embodiments, the compositions of the invention improve the functional endpoint in a subject with Alzheimer's disease. In preferred embodiments, the compositions of the invention improve the cognitive and functional endpoint in a subject with Alzheimer's disease. In yet further preferred embodiments, the compositions of the invention improve the overall clinical response (the global endpoint) in a subject with Alzheimer's disease.

In some embodiments, the compositions of the invention improve the symptoms of neurocognitive disorders according to a symptomatic or diagnostic test. In certain embodiments, the tests for assessing symptomatic improvement of Alzheimer's disease (and other neurocognitive disorders) are selected from objective cognitive, activities of daily living, global assessment of change, health related quality of life tests and tests assessing behavioural and psychiatric symptoms of neurocognitive disorders.

In certain embodiments, the objective cognitive tests for assessment of symptomatic improvement use the Alzheimer's disease Assessment Scale cognitive subscale (ADAS-cog) and the classic ADAS scale. In certain embodiments, symptomatic improvement of cognition is assessed using the Neurophysiological Test Battery for Use in Alzheimer's Disease (NTB).

In some embodiments, the global assessment of change test uses the Clinical Global Impression—Global Improvement (CGI-I) scale for assessing psychiatric and neurological disorders. In some embodiments, the global scale is the Clinician's Interview Based Impression of Change plus (CIBIC-plus). In some embodiments, the global scale is the Alzheimer's Disease Cooperative Study Unit Clinician's Global Impression of Change (ADCS-CGIC).

In certain embodiments, the health related quality of life measures are the Alzheimer's Disease-Related QOL (ADRQL) and the QOL-Alzheimer's Disease (QOL-AD).

In certain embodiments, the tests assessing behavioural and psychiatric symptoms of neurocognitive disorders are selected from the Behavioural pathology in Alzheimer's Disease Rating Scale (BEHAVE-AD); the Behavioural Rating Scale for Dementia (BRSD); the Neuropsychiatric Inventory (NPI); and the Cohen-Mansfield Agitation Inventory (CMAI).

In some embodiments, the compositions of the invention are particularly effective at preventing, reducing or alleviating neurocognitive disorders when used in combination with another therapy for treating neurocognitive disorders. In certain embodiments, such therapies include acetylcholinesterase inhibitors including donepezil (Aricept®), galantamine (Razadyne®) and rivastigmine (Exelon ®), and memantine.

### Parkinson's Disease

Parkinson's disease is a common neurodegenerative disease neuropathologically characterised by degeneration of heterogeneous populations of neural cells (dopamine-producing cells). The clinical diagnosis of Parkinson's disease requires bradykinesia and at least one of the following core symptoms: resting tremor; muscle rigidity and postural reflex impairment. Other signs and symptoms that may be present or develop during the progression of the disease are autonomic disturbances (sialorrhoea, seborrhoea, constipation, micturition disturbances, sexual functioning, orthostatic hypotension, hyperhydrosis), sleep disturbances and disturbances in the sense of smell or sense of temperature. Depressive symptoms and cognitive dysfunction comorbidities develop in many Parkinson's disease patients, as well as neurocognitive disorders related to Lewy Bodies.

Parkinson's disease is a psychiatric disorder that may develop or persist due to dysfunction of the microbiota-gut-brain axis. Therefore, in preferred embodiments, the compositions of the invention are for use in treating or preventing Parkinson's disease in a subject.

In preferred embodiments, the compositions of the invention prevent, reduce or alleviate one or more of the symptoms of Parkinson's disease in a subject. In preferred embodiments, the compositions of the invention prevent, reduce or alleviate one or more core symptoms of Parkinson's disease in a subject. In certain embodiments, the compositions of the invention prevent, reduce or alleviate bradykinesia in a subject. In certain embodiments, the compositions of the invention prevent, reduce or alleviate resting tremor; muscle rigidity and/or postural reflex impairment in a subject. In certain embodiments, the compositions of the invention prevent, reduce or alleviate one or more symptoms associated with Parkinson's disease progression selected from autonomic disturbances (sialorrhoea, seborrhoea, constipation, micturition disturbances, sexual functioning, orthostatic hypotension, hyperhydrosis), sleep disturbances and disturbances in the sense of smell or sense of temperature.

In preferred embodiments, the compositions of the invention prevent, reduce or alleviate depressive symptoms comorbid with Parkinson's disease. In certain embodiments, the compositions of the invention improve verbal memory and/or executive functions. In certain embodiments, the compositions of the invention improve attention, working memory, verbal fluency and/or anxiety.

In other preferred embodiments, the compositions of the invention prevent, reduce or alleviate cognitive dysfunctions comorbid with Parkinson's disease.

In certain embodiments, the compositions of the invention prevent, reduce or alleviate Parkinson's disease progression. In certain embodiments, the compositions of the invention prevent, reduce or alleviate later motor complications. In certain embodiments, the compositions of the invention prevent, reduce or alleviate late motor fluctuations. In certain embodiments, the compositions of the invention prevent, reduce or alleviate neuronal loss. In certain embodiments, the compositions of the invention improve symptoms of Parkinson's disease dementia (PDD). In certain embodiments, the compositions of the invention prevent, reduce or alleviate impairment of executive function, attention and/or working memory. In certain embodiments, the compositions of the invention improve dopaminergic neurotransmission. In certain embodiments, the compositions of the invention prevent, reduce or alleviate impaired dopaminergic neurotransmission.

In some embodiments, the compositions of the invention improve the symptoms of Parkinson's disease according to a symptomatic or diagnostic scale. In certain embodiments, the tests for assessing symptomatic improvement of motor function in Parkinson's disease is the Unified Parkinson's Disease Rating Scale. In particular, UPDRS II considers the activity of daily life and UPDRS III considers motor-examination.

In some embodiments, the compositions of the invention improve the symptoms associated the PDD according to a symptomatic or diagnostic test and/or scale. In certain embodiments, the test or scale is selected from the Hopkins Verbal Learning Test—Revised (HVLT-R); the Delis-Kaplan Executive Function System (D-KEFS) Color-Word Interference Test; the Hamilton Depression Rating Scale (HAM-D 17; depression); the Hamilton Anxiety Rating Scale (HAM-A; anxiety) and the Unified Parkinson's Disease Rating Scale (UPDRS; PD symptom severity).

In some embodiments, the compositions of the invention improve the Clinical Global Impression—Global Improvement (CGI-I) scale for assessing psychiatric and neurological disorders. In some embodiments, the compositions of the invention display a positive effect on global social and occupational impairment of the subject with Parkinson's disease.

In some embodiments, the compositions of the invention are particularly effective at preventing, reducing or alleviating neurocognitive disorders when used in combination with another therapy for treating neurocognitive disorders. In certain embodiments, such therapies include dopamine agonists (including L-Dopa+); monoamine oxidase inhibitors, catecholamine-O-methyl transferase inhibitors; anticholinergics and glutamate modulators.

### Other Central Nervous System Disorders

In preferred embodiments, the compositions of the invention are for use in treating or preventing a central nervous system disorder associated with dysfunction of the microbiota-gut-brain axis. In addition to the embodiments above, the compositions of the invention are for use in treating or preventing psychosis; chronic fatigue syndrome (myalgic encephalomyelitis) and/or chronic pain. In further embodiments, the compositions of the invention may be useful for treating or preventing motor neuron disease; Huntington's disease; Guillain-Barre syndrome and/or meningitis.

### Neurochemical Factors, Neuropeptides and Neurotransmitters and the Microbiota-Gut-Brain Axis

As outlined above, the microbiota-gut-brain axis is modulated by a number of different physiological systems. The microbiota-gut-brain axis is modulated by a number of signalling molecules. Alterations in the levels of these signalling molecules results in defects in central nervous system development and/or functionality. Indeed, many of the molecules disclosed in this section have been implicated in the functionality of the microbiota-gut-brain axis and the pathogenesis of central nervous system disorders or conditions ([10], [14], [38], [39]). The experiments performed by the inventors indicate that behavioural changes can be triggered by administration of Enterococcus faecium. This effect may be mediated by an effect on levels of the signalling molecules, in particular those listed in this section. These alterations may be responsible for the therapeutic benefits associated with Enterococcus faecium. Accordingly, due to the fact that the central nervous system disorders and conditions disclosed herein display a similar fundamental biochemical and physiological pathogenesis (i.e. via the microbiota-gut-brain axis), a similar therapeutic benefit of Enterococcus faecium may be also achieved for these disorders and conditions.

The signalling of the microbiota-gut-brain axis is modulated by levels of neurochemical factors, neuropeptides and neurotransmitters. Accordingly, in certain embodiments, the compositions of the invention modulates levels of neurochemical factors, neuropeptides and neurotransmitters.

Accordingly, in certain preferred embodiments, the compositions of the invention directly alter CNS biochemistry. In preferred embodiments, the compositions of the invention modulate the levels of brain-derived neurotrophic factor (BDNF). In certain embodiments, the compositions of the invention modulate the levels of monoamines. In certain embodiments, the monoamines are serotonin (5-hydroxytryptamine (5-HT)), dopamine, norepinephrine and/or epinephrine. In certain embodiments, the monoamines are catecholamines. In certain embodiments, the catecholamines are dopamine, norepinephrine and epinephrine. In certain embodiments, the monoamines are tryptamines. In certain embodiments, the tryptamines are serotonin and melatonin. In certain embodiments, the compositions of the invention modulate the levels of acetylcholine.

In certain preferred embodiments, the compositions of the invention modulate the levels of oxytoxin. Oxytocin is associated with emotional, social, cognitive and neuroendocrine physiologies as well as autoregulation. In particular, oxytocin release is involved in anxiolysis; positive mood; maternal behaviour, pair bonding; sexual behaviour; social memory; olfactory memory; anorexiant effects; attenuation of the HPA axis response to stress; autoexcitation during birth and suckling as well as other physiological and psychological processes. In certain embodiments, the compositions of the invention increase the levels of oxytocin. In certain embodiments, the compositions of the invention decrease the levels of oxytocin. In certain embodiments, the compositions of the invention increase or decrease oxytocin signalling. In certain embodiments, the compositions of the invention modulate the levels of oxytocin receptors. In certain embodiments, the compositions of the invention modulate the flux of calcium ions into or out of neuronal, muscle and gastrointestinal cells. In preferred embodiments, the compositions of the invention treat and prevent neurodevelopmental and neuropsychiatric disorders and diseases associated with the microbiota-gut-brain axis by modulating the levels of oxytocin.

In certain embodiments, the compositions of the invention modulate the levels of brain monoamines and metabolites thereof. In preferred embodiments, the monoamine is serotonin. In certain embodiments, the compositions of the invention modulate the serotonergic and/or kynurenine routes of tryptophan metabolism. In certain embodiments, the compositions of the invention modulate the levels of serotonin metabolites, such as 5-Hydroxyindoleacetic acid (5-HIAA). In certain embodiments, the compositions of the invention modulate the levels of dopamine metabolites, such as Homovanillic acid (HVA). Modulation of these neurotransmitters and neurochemical factors is useful for treating stress, depression and anxiety-related disorders.

The signalling of the microbiota-gut-brain axis is modulated by levels of γ-aminobutyric acid (GABA). Accordingly, in preferred embodiments, the compositions of the invention modulate the levels of GABA. GABA is an inhibitory neurotransmitter that reduces neuronal excitability. In certain embodiments, the compositions of the invention increase the levels of GABA. In certain embodiments, the compositions of the invention decrease the levels of GABA. In certain embodiments, the compositions of the invention alter GABAergic neurotransmission. In certain embodiments, the compositions of the invention modulate the level of GABA transcription in different regions of the central nervous system. In certain embodiments, the commensal derived GABA crosses the blood-brain barrier and affects neurotransmission directly. In certain embodiments, the compositions of the invention lead to a reduction of GABA in the hippocampus, amygdala and/or locus coeruleus. In certain embodiments, the compositions of the invention lead to an increase of GABA in cortical regions.

The levels of neuroactive molecules, such as serotonin, melatonin, GABA, histamines and acetylcholine are linked to the pathophysiology of central nervous system diseases such as dementia, Alzheimer's disease and Huntington's disease.

The signalling of the microbiota-gut-brain axis is modulated by levels of histamines. Accordingly, in certain embodiments, the compositions of the invention modulate the levels of histamines. In certain embodiments, the histamines has an immunoregulatory effect. In certain embodiments, histamine levels enable translocation of bacteria from the lumen into systemic circulation. Therefore, in some embodiments, the compositions of the invention alter gastrointestinal tract permeability and/or barrier function. In certain other embodiments, the histamine acts as a neurotransmitter linked to central processes.

The signalling of the microbiota-gut-brain axis is modulated by the HPA axis. Accordingly, in certain embodiments, the compositions of the invention modulate HPA activity. In certain embodiments, the compositions of the invention attenuate the HPA stress response. In certain preferred embodiments, the compositions of the invention modulate inflammatory responses associated with HPA activity. In certain embodiments, the compositions of the invention modulate the levels of glucocorticoids. In certain preferred embodiments, the compositions of the invention modulate the levels of corticosterone and adrenaline. In certain embodiments, the compositions of the invention modulate the levels of corticotrophin-releasing factor and/or vasopressin. In certain embodiments, the compositions of the invention modulate the levels of vasopressin and/or other neurohypophysial or antidiuretic hormones. Alterations in HPA axis activity are associated with anxiety and stress disorders.

The signalling of the microbiota-gut-brain axis is modulated by alterations in the immune response and inflammatory factors and markers. Accordingly, in certain embodiments, the compositions of the invention may modulate the immune response. In certain embodiments, the compositions of the invention modulate the systemic levels of circulating neuroimmune signalling molecules. In certain preferred embodiments, the compositions of the invention modulate pro-inflammatory cytokine production and inflammation. In certain embodiments, the compositions of the invention modulate the inflammatory state. In certain embodiments, the compositions of the invention modulate the splenocyte proliferative response. In certain embodiments, the compositions of the invention modulate the systemic and/or plasma levels of C-reactive protein; IL-1 family cytokines; IL-1β; IL-2; IL-4; IL-6; IL-8; IL-10; IL-12p40; IL-17; IL-17A; IL-21; IL-23; TNF-α and IFN-γ. In some embodiments the compositions of the invention module the levels of anti-inflammatory cytokines, for example IL-10. In preferred embodiments, the compositions of the invention increase the levels of IL-10. In some embodiments, the compositions of the invention modulate the levels of TNF-α. In preferred embodiments, the compositions of the invention modulate the levels of IFN-γ. In some embodiments, the compositions of the invention modulate the IFN-γ:IL-10 ratio. In certain preferred embodiments, the compositions of the invention decrease the IFN-γ:IL-10 ratio. In preferred embodiments, the compositions of the invention decrease the levels of the pro-inflammatory cytokines TNF-α and IFN-γ. Increased circulating levels of cytokines are closely associated with various neuropsychiatric disorders, including depression, anxiety, schizophrenia and ASD. Evidence of inflammatory state alteration is highlighted in disorders such as schizophrenia, major depressive disorder and bipolar disorder.

In certain embodiments, the compositions of the invention modulates the levels of tolerance-mediating dendritic cells and reciprocally regulate pro and anti-inflammatory cytokine responses. In certain embodiments, the compositions of the invention decrease the systemic level of myeloperoxidase (a marker for inflammation and oxidation).Therapeutic modulators of the immune system and of inflammatory responses are useful for treating autism spectrum disorders and mood disorders.

In certain embodiments, the compositions of the invention modulate the immune response to an infection or vaccination. In certain embodiments, the compositions of the invention modulate the level of inflammation in response to infection or vaccination. In certain preferred embodiments, the compositions of the invention modulate maternal immune activation in response to an infection or vaccination during pregnancy. Accordingly, the compositions of the invention can be administered during pregnancy in order to treat or prevent a central nervous system disorder in the offspring.

The signalling of the microbiota-gut-brain axis is modulated by levels commensal metabolites. Accordingly, in certain embodiments, the compositions of the invention modulate the systemic levels of microbiota metabolites. In certain preferred embodiments, the compositions of the invention modulate the level of short chain fatty acids (SCFAs). In certain embodiments the level of SCFAs is increased or decreased. In some embodiments, the SCFA is butyric acid (BA) (or butyrate). In some embodiments, the SCFA is propionic acid (PPA). In some embodiments, the SCFA is acetic acid. In certain embodiments, the compositions of the invention modulate the ability of SCFAs to cross the blood-brain barrier. In certain embodiments, the compositions of the invention modulate the level of Polysaccharide A (PSA). In certain embodiments, the compositions of the invention modulate the levels of the potent pro-inflammatory endotoxin lipopolysaccharide (LPS). LPS leads to the production of inflammatory cytokines that alter physiological brain activity and modulate neuropeptide synthesis. LPS has an important influence on the modulation of the CNS, increasing the activity of areas devoted to the control of emotions (e.g. the amygdala). In certain embodiments, the compositions of the invention modulate the level of tryptophan and/or its metabolites. In certain embodiments, the compositions of the invention modulate the levels of 4-ethylphenylsulphate (4EPS; a uremic toxic associated with ASD-related behavioural abnormalities). In preferred embodiments, the compositions of the invention decrease the levels of 4-ethylphenylsulphate in a subject. The signals generated by the stimulation of neuronal signalling pathways caused by intraluminal gut stimuli strongly modulate brain activity, including pain perception, immune-response modulation, emotional control and other homeostatic functions. Accordingly, a composition able to modulate levels of these factors would have broad therapeutic applications for treating or preventing CNS disorders.

The signalling of the microbiota-gut-brain axis is modulated by levels gastrointestinal permeability. Accordingly, in some embodiments, the compositions of the invention alter the integrity of the gastrointestinal tract epithelium. In certain embodiments, the compositions of the invention modulate the permeability of the gastrointestinal tract. In certain embodiments, the compositions of the invention modulate the barrier function and integrity of the gastrointestinal tract. In certain embodiments, the compositions of the invention modulate gastrointestinal tract motility. In certain embodiments, the compositions of the invention modulate the translocation of commensal metabolites and inflammatory signalling molecules into the bloodstream from the gastrointestinal tract lumen.

The signalling of the microbiota-gut-brain axis is modulated by microbiome composition in the gastrointestinal tract. Accordingly, in certain embodiments, the compositions of the invention modulates the microbiome composition of the gastrointestinal tract. In certain embodiments, the compositions of the invention prevents microbiome dysbiosis and associated increases in toxic metabolites (e.g. LPS). In certain embodiments, the compositions of the invention modulate the levels of Clostridium in the gastrointestinal tract. In preferred embodiments, the compositions of the invention reduce the level of Clostridium in the gastrointestinal tract. In certain embodiments, the compositions of the invention reduce the levels of Campylobacter jejuni. In certain embodiments, the compositions of the invention modulate the proliferation of harmful anaerobic bacteria and the production of neurotoxins produced by these bacteria. In certain embodiments, the compositions of the invention modulate the microbiome levels of Lactobacillus and/or Bifidobacterium. In certain embodiments, the compositions of the invention modulate the microbiome levels of Sutterella, Prevotella, Ruminoccucs genera and/or the Alcaligenaceae family. In certain embodiments, the compositions of the invention increase the level of Lactobacillus plantarum and/or Saccharomyces boulardii.

In certain embodiments, the compositions of the invention prevent the dysregulation of the composition of the microbiome by extensive antibiotic use. In certain preferred embodiments, the compositions of the invention maintain a functional maternal microbiome composition upon administration of antibiotics during pregnancy. Accordingly, the compositions of the invention can be administered during pregnancy in order to treat or prevent a central nervous system disorder in the offspring.

Modulation of the microbiome has been shown to be effective at improving psychiatric disorder-related behaviours, including anxiety, depression, autism spectrum disorder, obsessive-compulsive disorder and memory abilities (including spatial and non-spatial memory), as well as other CNS-related disorders including Parkinson's disease. Certain studies have suggested that probiotics can reduce psychological stress, somatisation, depression and anger-hostility. The levels of Lactobacillus are associated with depression and have been implicated in pain signalling associated with gastrointestinal discomfort.

In certain embodiments, the compositions of the invention prevent, reduce or alleviate at least one of the behavioural symptoms associated with a central nervous system disorder described herein. In preferred embodiments, the compositions of the invention improve the overall clinical response in a subj ect.

In preferred embodiments, the compositions of the invention prevent, reduce or alleviate stereotyped, repetitive behaviour in a subject. In preferred embodiments, the compositions of the invention prevent, reduce or alleviate the occurrence of unusually restrictive behaviours and/or interests. In certain embodiments, the compositions of the invention prevent, reduce or alleviate recurrent obsessions and/or compulsions in a subject. In preferred embodiments, the compositions of the invention prevent, reduce or alleviate deficits in social behaviour in a subject. In preferred embodiments, the compositions of the invention prevent, reduce or alleviate avoidance behaviour in a subject. In preferred embodiments, the compositions of the invention prevent, reduce or alleviate deficits in communication behaviour in a subject.

In preferred embodiments, the compositions of the invention prevent, reduce or alleviate negative alterations in cognitions and mood in a subject. In preferred embodiments, the compositions of the invention prevent, reduce or alleviate anxiety-related behaviour in a subject. In preferred embodiments, the compositions of the invention prevent, reduce or alleviate stress-related behaviour in a subject. In preferred embodiments, the compositions of the invention prevent, reduce or alleviate depression-related behaviour in a subject. In preferred embodiments, the compositions of the invention prevent, reduce or alleviate aggressive behaviour in a subject. In preferred embodiments, the compositions of the invention prevent, reduce or alleviate the occurrence of an abnormally and persistently elevated, expansive, or irritable mood.

In preferred embodiments, the compositions of the invention prevent, reduce or alleviate intrusive thoughts in a subject. In preferred embodiments, the compositions of the invention prevent alterations in arousal and reactivity in a subject. In preferred embodiments, the compositions of the invention prevent, reduce or alleviate delusions, hallucinations, disorganised speech, and disorganised or catatonic behaviours in a subject. In preferred embodiments, the compositions of the invention prevent, reduce or alleviate affective flattening, restriction in the fluency and productivity of thought and speech and in the initiation of goal directed behaviour in a subject. In preferred embodiments, the compositions of the invention prevent, reduce or alleviate one or more of the following symptoms: high self-esteem; reduced need for sleep; increase rate of speech; rapid jumping of ideas; easily distracted; an increased interest in goals or activities; psychomotor agitation; increased pursuit of activities with a high risk of danger.

In preferred embodiments, the compositions of the invention improve spatial and/or non-spatial memory deficits in a subject. In preferred embodiments, the compositions of the invention improve both cognition and functioning in a subject. In preferred embodiments, the compositions of the invention improve locomotor activity in a subject. In preferred embodiments, the compositions of the invention prevent, reduce or alleviate bradykinesia in a subject. In preferred embodiments, the compositions of the invention prevent, reduce or alleviate resting tremor; muscle rigidity and/or postural reflex impairment in a subject.

In preferred embodiments, the compositions of the invention prevent, reduce or alleviate at least one comorbidity associated with a CNS disorder disclosed herein.

In preferred embodiments, the compositions of the invention improve the scores of a subject on at least one of the symptomatic and/or diagnostic scales for CNS disorders described herein. In certain other embodiments, the symptomatic and/or diagnostic scale is selected from the General Health Questionnaire (GHQ); the Depression Anxiety and Stress Scale (DASS); the Leiden Index of Depression Sensitivity-Revised (LEIDS-r); the Positive and Negative Symptom Scale (PANSS); the State-Trait Anxiety Inventory (STAI); the Development Behavior Checklist (DBC); the Beck Depression Inventory (BDI); the Beck Anxiety Inventory (BAI); the Hopkins Symptom Checklist (HSCL-90); the Hospital Anxiety and Depression Scale (HADS); the Perceived Stress Scale (PSS); the Coping Checklist (CCL) (also used to counter the stress of daily life); and the questionnaire-based Profile of Mood State (POMS).

In certain embodiments, the compositions of the invention may improve the symptomatic and/or diagnostic scale when assessing therapeutic efficacy in other animal models of CNS disorders known to a person skilled in the art. In addition to the behavioural assays disclosed in the examples, the compositions of the invention may improve reciprocal social interactions; olfactory communication; ultrasonic vocalisation; motor stereotypes (such as circling and vertical jumping), repetitive behaviour such as self-grooming and diffing; and perseverance in spatial tasks.

In addition, the compositions of the invention will be useful in treating and/or preventing CNS disorders in other animal models of CNS disorders. Other mouse models include inbred mice strains (including BALB/cJ and C58/J) and also genetically modified mice strains (including NEUREXIN1, NEUROLIGIN3, NEUROLIGIN4, SHANK2, SHANKS, CNTNAP2, Tsc1/2 and Fmr1 gene mutant mice strains).

In certain embodiments, the compositions of the invention improve social behaviour of a subject. In preferred embodiments, the compositions of the invention improve the recognition of social novelty in a subject. In preferred embodiments, the compositions of the invention improve the ability to discriminate between familiar and novel objects and familiar and novel subjects. In preferred embodiments, the composition of the invention improve ability to recognise other subjects.

In certain embodiments, the compositions of the invention regulate plasma levels of amino acids. In certain embodiments, the compositions of the invention regulate the biosynthesis or catabolism of amino acids. In preferred embodiments, the compositions of the invention regulate plasma levels of proline. In preferred embodiments, the compositions of the invention reduce the plasma levels of proline. Elevated proline is known to negatively affect brain function by an increase in dopamine in the prefrontal cortex [40]. In addition, proline is considered to be a neurotransmitter that modulates glutamatergic neurotransmission in the hippocampus, and neurotransmission elsewhere in the brain. Accordingly, proline has been implicated in CNS disorders and psychiatric disorders, in particular psychosis. In preferred embodiments, the reduction in plasma levels of proline treats or prevents CNS disorders, in particular, ADHD, OCD, mood disorders, autism spectrum disorder, psychosis and schizophrenia.

In certain embodiments, the compositions of the invention prevent, reduce or alleviate the symptoms of psychiatric disorders, for example schizophrenia and bipolar disorder, associated with 22q11.2 deletion syndrome (22q11DS) [40]. In certain embodiments, the compositions of the invention improve the social behavioural and social cognitive problems in subjects with 22q11DS. In preferred embodiments, the compositions of the invention modulate the associated cognitive and behavioural outcomes in 22q11DS subjects. In preferred embodiments, the modulation of these outcomes is a consequence of reduced plasma levels of proline. In certain embodiments, the compositions of the invention modulate the activity of proline hydrogenase.

In certain embodiments, the compositions of the invention modulate the levels of NMDA receptors and/or the subunits thereof. In preferred embodiments, the compositions of the invention modulate the levels of the NMDA receptor 2B. In certain embodiments, the compositions of the invention increase the levels of the NMDA receptor 2B. In preferred embodiments, the compositions of the invention decrease the levels of the NMDA receptor 2B. Dysregulation of NMDA receptors have been associated with CNS disorders, in particular ASD and schizophrenia. There have been suggestions that NMDA receptor antagonists may be effective in treating ASD [41]. In addition, suppression of NMDA receptor function has been demonstrated to improve social deficits and reduce repetitive behaviour in valproic acid induced models of ASD [42]. In certain embodiments, the compositions of the invention cause hypofunction of the NMDA receptor 2B. In certain embodiments, the compositions of the invention cause hyperfunction of the NMDA receptor 2B. In certain embodiments, the compositions of the invention prevent, reduce or alleviate the symptoms of CNS disorders, for example ASD or schizophrenia as a consequence of the modulation of NMDA receptor 2B activity. In preferred embodiments, the compositions of the inventions suppress NMDA receptor activity and reduce social deficits and stereotypical behaviour in subjects with CNS disorders.

In certain embodiments, the compositions of the invention modulate the levels of BDNF. In preferred embodiments, the compositions of the invention reduce the levels of BDNF. In certain embodiments, the reduction in BDNF is localised to the amygdalar. Meta-analyses of ASD populations have shown that higher levels of BDNF are detected in ASD subjects compared to controls [43]. In preferred embodiments, the compositions of the invention prevent, reduce or alleviate the symptoms of CNS disorders, in particular ASD, as a consequence of the reduction in levels of BDNF. Altered levels of BDNF have been associated with a number of neurodevelopmental disorders, as well as psychosis and schizophrenia. In certain embodiments, the compositions of the invention modulate levels of BDNF in order to prevent, reduce or alleviate the symptoms of neurodevelopmental and psychiatric disorders.

In certain embodiments, the compositions of the invention modulate the levels of inflammatory markers produced in response to an antigen challenge. In preferred embodiments, the compositions of the invention increase the levels of IL-1β in response to a viral antigen challenge. In certain embodiments, the compositions of the invention modulate the innate immune response. In certain embodiments, the compositions of the invention modulate the adaptive immune response. In certain embodiments, the compositions of the invention modulate the inflammatory response.

### Modes of Administration

Preferably, the compositions of the invention are to be administered to the gastrointestinal tract in order to enable delivery to and/or partial or total colonisation of the intestine with the bacterial strain of the invention. Generally, the compositions of the invention are administered orally, but they may be administered rectally, intranasally, or via buccal or sublingual routes.

In certain embodiments, the compositions of the invention may be administered as a foam, as a spray or a gel.

In certain embodiments, the compositions of the invention may be administered as a suppository, such as a rectal suppository, for example in the form of a theobroma oil (cocoa butter), synthetic hard fat (e.g. suppocire, witepsol), glycero-gelatin, polyethylene glycol, or soap glycerin composition.

In certain embodiments, the composition of the invention is administered to the gastrointestinal tract via a tube, such as a nasogastric tube, orogastric tube, gastric tube, jejunostomy tube (J tube), percutaneous endoscopic gastrostomy (PEG), or a port, such as a chest wall port that provides access to the stomach, jejunum and other suitable access ports.

The compositions of the invention may be administered once, or they may be administered sequentially as part of a treatment regimen. In certain embodiments, the compositions of the invention are to be administered daily.

In certain embodiments of the invention, treatment according to the invention is accompanied by assessment of the patient's gut microbiota. Treatment may be repeated if delivery of and/or partial or total colonisation with the strain of the invention is not achieved such that efficacy is not observed, or treatment may be ceased if delivery and/or partial or total colonisation is successful and efficacy is observed.

In certain embodiments, the composition of the invention may be administered to a pregnant animal, for example a mammal such as a human in order to prevent an inflammatory or autoimmune disease developing in her child in utero and/or after it is born.

The compositions of the invention may be administered to a patient that has been diagnosed with a central nervous system disorder or condition, in particular a central nervous system disorder or condition mediated by the microbiota-gut-brain axis, or that has been identified as being at risk of a central nervous system disorder or condition, in particular central nervous system disorder or condition mediated by the microbiota-gut-brain axis. The compositions may also be administered as a prophylactic measure to prevent the development of central nervous system disorders or conditions, in particular central nervous system disorders or conditions mediated by the microbiota-gut-brain axis in a healthy patient.

The compositions of the invention may be administered to a patient that has been identified as having an abnormal gut microbiota. For example, the patient may have reduced or absent colonisation by Enterococcus faecium.

The compositions of the invention may be administered as a food product, such as a nutritional supplement.

Generally, the compositions of the invention are for the treatment of humans, although they may be used to treat animals including monogastric mammals such as poultry, pigs, cats, dogs, horses or rabbits. The compositions of the invention may be useful for enhancing the growth and performance of animals. If administered to animals, oral gavage may be used.

### Compositions

Generally, the composition of the invention comprises bacteria. In preferred embodiments of the invention, the composition is formulated in freeze-dried form. For example, the composition of the invention may comprise granules or gelatin capsules, for example hard gelatin capsules, comprising a bacterial strain of the invention.

Preferably, the composition of the invention comprises lyophilised bacteria. Lyophilisation of bacteria is a well-established procedure and relevant guidance is available in, for example, references [44], [ ], and [46].

Alternatively, the composition of the invention may comprise a live, active bacterial culture.

In some embodiments, the bacterial strain in the composition of the invention has not been inactivated, for example, has not been heat-inactivated. In some embodiments, the bacterial strain in the composition of the invention has not been killed, for example, has not been heat-killed. In some embodiments, the bacterial strain in the composition of the invention has not been attenuated, for example, has not been heat-attenuated. For example, in some embodiments, the bacterial strain in the composition of the invention has not been killed, inactivated and/or attenuated. For example, in some embodiments, the bacterial strain in the composition of the invention is live. For example, in some embodiments, the bacterial strain in the composition of the invention is viable. For example, in some embodiments, the bacterial strain in the composition of the invention is capable of partially or totally colonising the intestine. For example, in some embodiments, the bacterial strain in the composition of the invention is viable and capable of partially or totally colonising the intestine.

In some embodiments, the composition comprises a mixture of live bacterial strains and bacterial strains that have been killed.

In preferred embodiments, the composition of the invention is encapsulated to enable delivery of the bacterial strain to the intestine. Encapsulation protects the composition from degradation until delivery at the target location through, for example, rupturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule. Guidance on encapsulation that may be useful for preparing compositions of the invention is available in, for example, references [47] and [48].

The composition may be administered orally and may be in the form of a tablet, capsule or powder. Encapsulated products are preferred because Enterococcus faecium are anaerobes. Other ingredients (such as vitamin C, for example), may be included as oxygen scavengers and prebiotic substrates to improve the delivery and/or partial or total colonisation and survival in vivo. Alternatively, the probiotic composition of the invention may be administered orally as a food or nutritional product, such as milk or whey based fermented dairy product, or as a pharmaceutical product.

The composition may be formulated as a probiotic.

A composition of the invention includes a therapeutically effective amount of a bacterial strain of the invention. A therapeutically effective amount of a bacterial strain is sufficient to exert a beneficial effect upon a patient. A therapeutically effective amount of a bacterial strain may be sufficient to result in delivery to and/or partial or total colonisation of the patient's intestine.

A suitable daily dose of the bacteria, for example for an adult human, may be from about 1×10³ to about 1×10¹¹ colony forming units (CFU); for example, from about 1×10⁷ to about 1×10¹⁰ CFU; in another example from about 1×10⁶ to about 1×10¹⁰ CFU.

In certain embodiments, the composition contains the bacterial strain in an amount of from about 1×10⁶ to about 1×10¹¹ CFU/g, respect to the weight of the composition; for example, from about 1×10⁸ to about 1×10¹⁰ CFU/g. The dose may be, for example, 1 g, 3 g, 5 g, and 10 g.

Typically, a probiotic, such as the composition of the invention, is optionally combined with at least one suitable prebiotic compound. A prebiotic compound is usually a non-digestible carbohydrate such as an oligo- or polysaccharide, or a sugar alcohol, which is not degraded or absorbed in the upper digestive tract. Known prebiotics include commercial products such as inulin and transgalacto-oligosaccharides.

In certain embodiments, the probiotic composition of the present invention includes a prebiotic compound in an amount of from about 1 to about 30% by weight, respect to the total weight composition, (e.g. from 5 to 20% by weight). Carbohydrates may be selected from the group consisting of: fructo- oligosaccharides (or FOS), short-chain fructo-oligosaccharides, inulin, isomalt-oligosaccharides, pectins, xylo-oligosaccharides (or XOS), chitosan-oligosaccharides (or COS), beta-glucans, arable gum modified and resistant starches, polydextrose, D-tagatose, acacia fibers, carob, oats, and citrus fibers. In one aspect, the prebiotics are the short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded.

In certain embodiments, the compositions of the invention are used in combination with another therapeutic compound for treating or preventing the central nervous system disorder. In some embodiments, the compositions of the invention are administered with nutritional supplements that modulate central neurotransmitters and neuropeptides. In preferred embodiments, the nutritional supplements comprise or consist of nutritional vitamins. In certain embodiments, the vitamins are vitamin B6, magnesium, dimethylglycine (vitamin B16) and vitamin C. In certain embodiments, the compositions of the invention are administered in combination with another probiotic. In certain preferred embodiments, the probiotic comprises or consists of Trichuris suis ova.

The compositions of the invention may comprise pharmaceutically acceptable excipients or carriers. Examples of such suitable excipients may be found in the reference [49]. Acceptable carriers or diluents for therapeutic use are well known in the pharmaceutical art and are described, for example, in reference [50]. Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

The compositions of the invention may be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. In certain embodiments, the composition of the invention is formulated as a milk-based product. The term “milk-based product” means any liquid or semi-solid milk- or whey-based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Another important group includes milk beverages, such as whey beverages, fermented milks, condensed milks, infant or baby milks; flavoured milks, ice cream; milk-containing food such as sweets.

In some embodiments, the compositions of the invention comprise one or more bacterial strains of the species Enterococcus faecium and do not contain bacteria from any other species, or which comprise only de minimis or biologically irrelevant amounts of bacteria from another species. Thus, in some embodiments, the invention provides a composition comprising one or more bacterial strains of the species Enterococcus faecium, which does not contain bacteria from any other species or which comprises only de minimis or biologically irrelevant amounts of bacteria from another species, for use in therapy.

In some embodiments, the compositions of the invention comprise one or more bacterial strains of the species Enterococcus faecium and do not contain bacteria from any other Enterococcus species, or which comprise only de minimis or biologically irrelevant amounts of bacteria from another Enterococcus species. Thus, in some embodiments, the invention provides a composition comprising one or more bacterial strains of the species Enterococcus faecium, which does not contain bacteria from any other Enterococcus species or which comprises only de minimis or biologically irrelevant amounts of bacteria from another Enterococcus species, for use in therapy.

In certain embodiments, the compositions of the invention contain a single bacterial strain or species and do not contain any other bacterial strains or species. Such compositions may comprise only de minimis or biologically irrelevant amounts of other bacterial strains or species. Such compositions may be a culture that is substantially free from other species of organism.

In some embodiments, the invention provides a composition comprising a single bacterial strain of the species Enterococcus faecium, which does not contain bacteria from any other strains or which comprises only de minimis or biologically irrelevant amounts of bacteria from another strain for use in therapy.

In some embodiments, the compositions of the invention comprise more than one bacterial strain. For example, in some embodiments, the compositions of the invention comprise more than one strain from within the same species (e.g. more than 1, 2, 3, 4, 5, 6, 7, 8, 9, 10, 15, 20, 25, 30, 35, 40 or 45 strains), and, optionally, do not contain bacteria from any other species. In some embodiments, the compositions of the invention comprise less than 50 strains from within the same species (e.g. less than 45, 40, 35, 30, 25, 20, 15, 12, 10, 9, 8, 7, 6, 5, 4 or 3 strains), and, optionally, do not contain bacteria from any other species. In some embodiments, the compositions of the invention comprise 1-40, 1-30, 1-20, 1-19, 1-18, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same species and, optionally, do not contain bacteria from any other species. The invention comprises any combination of the foregoing.

In some embodiments, the composition comprises a microbial consortium. For example, in some embodiments, the composition comprises the Enterococcus faecium bacterial strain as part of a microbial consortium. For example, in some embodiments, the Enterococcus faecium bacterial strain is present in combination with one or more (e.g. at least 2, 3, 4, 5, 10, 15 or 20) other bacterial strains from other genera with which it can live symbiotically in vivo in the intestine. For example, in some embodiments, the composition comprises a bacterial strain of Enterococcus faecium in combination with a bacterial strain from a different genus. In some embodiments, the microbial consortium comprises two or more bacterial strains obtained from a faeces sample of a single organism, e.g. a human. In some embodiments, the microbial consortium is not found together in nature. For example, in some embodiments, the microbial consortium comprises bacterial strains obtained from faeces samples of at least two different organisms. In some embodiments, the two different organisms are from the same species, e.g. two different humans. In some embodiments, the two different organisms are an infant human and an adult human. In some embodiments, the two different organisms are a human and a non-human mammal.

In some embodiments, the composition of the invention additionally comprises a bacterial strain that has the same safety and therapeutic efficacy characteristics as strain MRX010, but which is not MRX010 deposited as NCIMB 42487, or which is not a Enterococcus faecium.

In some embodiments, the composition of the invention does not comprise a bacterial strain of the genus Bacillus. In some embodiments, the composition of the invention does not comprise Bacillus subtilis and/or does not comprise Bacillus coagulans. In some embodiments, the CNS disorder to be treated by the composition of the invention is not bipolar disorder. In some embodiments, the patient to be treated by the composition of the invention does not have a fungal infection. In some embodiments, the patient to be treated by the composition of the invention does not suffer from candidiasis. In some embodiments, the patient to be treated by the composition of the invention has not been diagnosed as having a fungal infection and/or has not been diagnosed as suffering from candidiasis. In preferred such embodiments, the patient to be treated by the composition of the invention has never been diagnosed as having a fungal infection and/or has never been diagnosed as suffering from candidiasis.

In some embodiments in which the composition of the invention comprises more than one bacterial strain, species or genus, the individual bacterial strains, species or genera may be for separate, simultaneous or sequential administration. For example, the composition may comprise all of the more than one bacterial strain, species or genera, or the bacterial strains, species or genera may be stored separately and be administered separately, simultaneously or sequentially. In some embodiments, the more than one bacterial strains, species or genera are stored separately but are mixed together prior to use.

In some embodiments, the bacterial strain for use in the invention is obtained from human adult faeces. In some embodiments in which the composition of the invention comprises more than one bacterial strain, all of the bacterial strains are obtained from human adult faeces or if other bacterial strains are present they are present only in de minimis amounts. The bacteria may have been cultured subsequent to being obtained from the human adult faeces and being used in a composition of the invention.

As mentioned above, in some embodiments, the one or more Enterococcus faecium bacterial strains is/are the only therapeutically active agent(s) in a composition of the invention. In some embodiments, the bacterial strain(s) in the composition is/are the only therapeutically active agent(s) in a composition of the invention.

The compositions for use in accordance with the invention may or may not require marketing approval.

In certain embodiments, the invention provides the above pharmaceutical composition, wherein said bacterial strain is lyophilised. In certain embodiments, the invention provides the above pharmaceutical composition, wherein said bacterial strain is spray dried. In certain embodiments, the invention provides the above pharmaceutical composition, wherein the bacterial strain is lyophilised or spray dried and wherein it is live. In certain embodiments, the invention provides the above pharmaceutical composition, wherein the bacterial strain is lyophilised or spray dried and wherein it is viable. In certain embodiments, the invention provides the above pharmaceutical composition, wherein the bacterial strain is lyophilised or spray dried and wherein it is capable of partially or totally colonising the intestine. In certain embodiments, the invention provides the above pharmaceutical composition, wherein the bacterial strain is lyophilised or spray dried and wherein it is viable and capable of partially or totally colonising the intestine.

In some cases, the lyophilised or spray dried bacterial strain is reconstituted prior to administration. In some cases, the reconstitution is by use of a diluent described herein.

The compositions of the invention can comprise pharmaceutically acceptable excipients, diluents or carriers.

In certain embodiments, the invention provides a pharmaceutical composition comprising: a bacterial strain as used in the invention; and a pharmaceutically acceptable excipient, carrier or diluent; wherein the bacterial strain is in an amount sufficient to treat a disorder when administered to a subject in need thereof; and wherein the disorder is selected from the group consisting of: autism spectrum disorders (ASDs); child developmental disorder; obsessive compulsive disorder (OCD); major depressive disorder; depression; seasonal affective disorder; anxiety disorders; schizophrenia spectrum disorders; schizophrenia; bipolar disorder; psychosis; mood disorder; chronic fatigue syndrome (myalgic encephalomyelitis); stress disorder; post-traumatic stress disorder; dementia; Alzheimer's; Parkinson's disease; and/or chronic pain; motor neuron disease; Huntington's disease; Guillain-Barre syndrome and/or meningitis.

In certain embodiments, the invention provides pharmaceutical composition comprising: a bacterial strain as used in the invention; and a pharmaceutically acceptable excipient, carrier or diluent; wherein the bacterial strain is in an amount sufficient to treat or prevent a central nervous system disorder or condition, in particular central nervous system disorder or condition mediated by the microbiota-gut-brain axis. In preferred embodiments, said disease or condition is selected from the group consisting of: autism spectrum disorders (ASDs); child developmental disorder; obsessive compulsive disorder (OCD); major depressive disorder; depression; seasonal affective disorder; anxiety disorders; schizophrenia spectrum disorders; schizophrenia; bipolar disorder; psychosis; mood disorder; chronic fatigue syndrome (myalgic encephalomyelitis); stress disorder; post-traumatic stress disorder; dementia; Alzheimer's; Parkinson's disease; and/or chronic pain. In further embodiments, the compositions of the invention may be useful for treating or preventing motor neuron disease; Huntington's disease; Guillain-Barre syndrome and/or meningitis.

In certain embodiments, the invention provides the above pharmaceutical composition, wherein the amount of the bacterial strain is from about 1×10³ to about 1×10¹¹ colony forming units per gram with respect to a weight of the composition.

In certain embodiments, the invention provides the above pharmaceutical composition, wherein the composition is administered at a dose of 1 g, 3 g, 5 g or 10 g.

In certain embodiments, the invention provides the above pharmaceutical composition, wherein the composition is administered by a method selected from the group consisting of oral, rectal, subcutaneous, nasal, buccal, and sublingual.

In certain embodiments, the invention provides the above pharmaceutical composition, comprising a carrier selected from the group consisting of lactose, starch, glucose, methyl cellulose, magnesium stearate, mannitol and sorbitol.

In certain embodiments, the invention provides the above pharmaceutical composition, comprising a diluent selected from the group consisting of ethanol, glycerol and water.

In certain embodiments, the invention provides the above pharmaceutical composition, comprising an excipient selected from the group consisting of starch, gelatin, glucose, anhydrous lactose, free-flow lactose, beta-lactose, corn sweetener, acacia, tragacanth, sodium alginate, carboxymethyl cellulose, polyethylene glycol, sodium oleate, sodium stearate, magnesium stearate, sodium benzoate, sodium acetate and sodium chloride.

In certain embodiments, the invention provides the above pharmaceutical composition, further comprising at least one of a preservative, an antioxidant and a stabilizer.

In certain embodiments, the invention provides the above pharmaceutical composition, comprising a preservative selected from the group consisting of sodium benzoate, sorbic acid and esters of p-hydroxybenzoic acid.

In certain embodiments, the invention provides the above pharmaceutical composition, wherein when the composition is stored in a sealed container at about 4.0 or about 25.0 and the container is placed in an atmosphere having 50% relative humidity, at least 80% of the bacterial strain as measured in colony forming units, remains after a period of at least about: 1 month, 3 months, 6 months, 1 year, 1.5 years, 2 years, 2.5 years or 3 years.

In some embodiments, the composition of the invention is provided in a sealed container comprising a composition as described herein. In some embodiments, the sealed container is a sachet or bottle. In some embodiments, the composition of the invention is provided in a syringe comprising a composition as described herein.

The composition of the present invention may, in some embodiments, be provided as a pharmaceutical formulation. For example, the composition may be provided as a tablet or capsule. In some embodiments, the capsule is a gelatine capsule (“gel-cap”).

In some embodiments, the compositions of the invention are administered orally. Oral administration may involve swallowing, so that the compound enters the gastrointestinal tract, and/or buccal, lingual, or sublingual administration by which the compound enters the blood stream directly from the mouth. Pharmaceutical formulations suitable for oral administration include solid plugs, solid microparticulates, semi-solid and liquid (including multiple phases or dispersed systems) such as tablets; soft or hard capsules containing multi- or nano-particulates, liquids (e.g. aqueous solutions), emulsions or powders; lozenges (including liquid-filled); chews; gels; fast dispersing dosage forms; films; ovules; sprays; and buccal/mucoadhesive patches.

In some embodiments the pharmaceutical formulation is an enteric formulation, i.e. a gastro-resistant formulation (for example, resistant to gastric pH) that is suitable for delivery of the composition of the invention to the intestine by oral administration. Enteric formulations may be particularly useful when the bacteria or another component of the composition is acid-sensitive, e.g. prone to degradation under gastric conditions.

In some embodiments, the enteric formulation comprises an enteric coating. In some embodiments, the formulation is an enteric-coated dosage form. For example, the formulation may be an enteric-coated tablet or an enteric-coated capsule, or the like. The enteric coating may be a conventional enteric coating, for example, a conventional coating for a tablet, capsule, or the like for oral delivery. The formulation may comprise a film coating, for example, a thin film layer of an enteric polymer, e.g. an acid-insoluble polymer.

In some embodiments, the enteric formulation is intrinsically enteric, for example, gastro-resistant without the need for an enteric coating. Thus, in some embodiments, the formulation is an enteric formulation that does not comprise an enteric coating. In some embodiments, the formulation is a capsule made from a thermogelling material. In some embodiments, the thermogelling material is a cellulosic material, such as methylcellulose, hydroxymethylcellulose or hydroxypropylmethylcellulose (HPMC). In some embodiments, the capsule comprises a shell that does not contain any film forming polymer. In some embodiments, the capsule comprises a shell and the shell comprises hydroxypropylmethylcellulose and does not comprise any film forming polymer (e.g. see [51]). In some embodiments, the formulation is an intrinsically enteric capsule (for example, Vcaps® from Capsugel).

In some embodiments, the formulation is a soft capsule. Soft capsules are capsules which may, owing to additions of softeners, such as, for example, glycerol, sorbitol, maltitol and polyethylene glycols, present in the capsule shell, have a certain elasticity and softness. Soft capsules can be produced, for example, on the basis of gelatine or starch. Gelatine-based soft capsules are commercially available from various suppliers. Depending on the method of administration, such as, for example, orally or rectally, soft capsules can have various shapes, they can be, for example, round, oval, oblong or torpedo-shaped. Soft capsules can be produced by conventional processes, such as, for example, by the Scherer process, the Accogel process or the droplet or blowing process.

### Culturing Methods

The bacterial strains for use in the present invention can be cultured using standard microbiology techniques as detailed in, for example, references [52]-[54].

The solid or liquid medium used for culture may be YCFA agar or YCFA medium. YCFA medium may include (per 100 ml, approximate values): Casitone (1.0 g), yeast extract (0.25 g), NaHCO₃ (0.4 g), cysteine (0.1 g), K₂HPO₄ (0.045 g), KH₂PO₄ (0.045 g), NaCl (0.09 g), (NH₄)₂SO₄ (0.09 g), MgSO₄.7H₂O (0.009 g), CaCl₂ (0.009 g), resazurin (0.1 mg), hemin (1 mg), biotin (1 μg), cobalamin (1 μg), p-aminobenzoic acid (3 μg), folic acid (5 μg), and pyridoxamine (15 μg).

### Bacterial Strains for Use in Vaccine Compositions

The inventors have identified that the bacterial strains of the invention are useful for treating or preventing central nervous system disorders or conditions, in particular central nervous system disorders or conditions mediated by the microbiota-gut-brain axis. This is likely to be a result of the effect that the bacterial strains of the invention have on the host central, autonomic and/or enteric nervous system; the activity of the HPA pathway; the neuroimmune and neuroendocrine pathways; and the level of commensal metabolites in the host gastrointestinal tract and/or gastrointestinal permeability of the host. Therefore, the compositions of the invention may also be useful for preventing central nervous system disorders or conditions, in particular central nervous system disorders or conditions mediated by the microbiota-gut-brain axis, when administered as vaccine compositions. In certain such embodiments, the bacterial strains of the invention are viable. In certain such embodiments, the bacterial strains of the invention are capable of partially or totally colonising the intestine. In certain such embodiments, the bacterial strains of the invention are viable and capable of partially or totally colonising the intestine. In other certain such embodiments, the bacterial strains of the invention may be killed, inactivated or attenuated. In certain such embodiments, the compositions may comprise a vaccine adjuvant. In certain embodiments, the compositions are for administration via injection, such as via subcutaneous injection.

### General

The practice of the present invention will employ, unless otherwise indicated, conventional methods of chemistry, biochemistry, molecular biology, immunology and pharmacology, within the skill of the art. Such techniques are explained fully in the literature. See, e.g., references [55] and [56]-[62], etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

In certain embodiments the term “modulate” means increase or activate. In alternative embodiments, the term “modulate” means decrease or suppress.

The word “substantially” does not exclude “completely” e.g. a composition which is “substantially free” from Y may be completely free from Y. Where necessary, the word “substantially” may be omitted from the definition of the invention.

References to a percentage sequence identity between two nucleotide sequences means that, when aligned, that percentage of nucleotides are the same in comparing the two sequences. This alignment and the percent homology or sequence identity can be determined using software programs known in the art, for example those described in section 7.7.18 of ref. [63]. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref. [64].

Unless specifically stated, a process or method comprising numerous steps may comprise additional steps at the beginning or end of the method, or may comprise additional intervening steps. Also, steps may be combined, omitted or performed in an alternative order, if appropriate.

Various embodiments of the invention are described herein. It will be appreciated that the features specified in each embodiment may be combined with other specified features, to provide further embodiments. In particular, embodiments highlighted herein as being suitable, typical or preferred may be combined with each other (except when they are mutually exclusive).

### Modes for Carrying Out the Invention

The present study aimed to assess the effect of live biotherapeutics on the treatment of central nervous system disorders or conditions in two different mouse models that display behavioural characteristics associated with neurodevelopmental and psychiatric disorders. In particular, the study focuses on autistic-related behaviour in (i) a maternal immune activation (MIA) mouse model and (ii) a black and tan, brachyuric (BTBR) genetically modified, inbred mouse model. The effects of chronic MRX010 versus vehicle treatment across anxiety, depression, and cognitive and social domains of behaviour in the two mouse model were investigated.

### EXAMPLE 1

### The Maternal Immune Activation (MIA) Mouse Model

The MIA mouse model uses an environmental immune challenge in pregnant mice in order to trigger the core symptoms of autism spectrum disorder in the offspring. MIA mice typically display stereotyped behaviour (as shown by the grooming and marble burying tests) and deficits in social communication (as shown by the social play, 3-chamber social interaction, and social transmission of food preference tests). The offspring display the three core symptoms of autism (reduced communication; reduced sociability; and increased repetitive or stereotyped behaviour) and therefore provide a suitable model in which to determine whether administration of a therapeutic can alleviate the behavioural phenotypes associated with autistic spectrum disorders and indeed in a number of other neurological disorders. It is well established that alteration of behavioural phenotypes in animal models is indicative of a potentially clinically relevant intervention, irrespective of an understanding of the underlying biological or physiological mechanism ([65]).

### EXAMPLE 1a

### Materials and Methods for MIA Mouse Model

### Mice

Maternal immune activation (environmental ASD mouse model) protocol was conducted as previously described in [66]Error! Bookmark not defined. Briefly, pregnant C57BL/6N mice (ENVIGO, UK) were injected i.p. on E12.5 with saline or 20 mg/kg poly(I:C) according to methods described in [66]. These mice are listed in the experiments below as MIA mice. Male mice started behaviour at 8 weeks old. The animals were housed in a temperature- and humidity-controlled room on a 12 hr dark cycle (lights on from 7:00-19:00 hr). All experiments were conducted in accordance with the European Directive 2010/63/EEC, the requirements of S.I. No 543 of 2012, and approved by the Animal Experimentation Ethics Committee of University College Cork.

### Strain

MRX010: Enterococcus faecium, bacteria deposited under accession number NCIMB 42487.

Live biotherapeutics were grown in the facility in anaerobic conditions.

### Live Biotherapeutic Administration

Dosing with MRX010 or vehicle commenced when the mice were 8 weeks old. These mice were treated once daily with MRX010 or phosphate buffer solution (PBS) for 3 weeks before the beginning of the behavioural battery. Mice were further treated once daily for 7 weeks during the behavioural battery. MRX010 (1×107 to 1×109 CFU oral administration) was dissolved in PBS prior to administration.

### Administration Schedule

The treatment groups for the study are shown below. The vehicle for oral administration is PBS. Daily oral administration occurs via oral gavage.

| Group | Treatment | Number |
|---|---|---|
| 1 | Control (PBS, oral gavage) | 11 |
| 2 | Vehicle MIA (PBS, oral gavage) | 10 |
| 3 | MRX010 MIA (oral gavage in PBS) | 11 |

### Fecal Collection

Fresh fecal samples were collected from individual mice every week until the end of the study. At least 20 mg of fresh faeces were placed in a microcentrifuge tube, place immediately on ice and then stored at −80° C.

### Experimental Design and Methods

As outlined above, dosing with MRX010 or vehicle commenced when the mice were 8 weeks old. The behavioural battery occurred in the following order: marble burying test at week 5; social transmission of food preference at week 6 and the forced swimming test at week 8. The carmine red gastrointestinal motility assay and gastrointestinal permeability assay tail bleeds occurred during weeks 7 and 8 respectively. Finally, in week 9, the mice were killed for splenocyte stimulation and ex vivo measurement of FITC in the ileum and colon.

The effects of live biotherapeutic treatment in the MIA model on stereotyped, social and depression-like behaviours, along with gastrointestinal parameters (permeability and motility) are outlined in the following examples.

Group 2, listed in the table above, represents the maternal immune activation mice, the mothers of which were treated with poly (I:C) during pregnancy. These mice would be expected to show phenotypes associated with autistic spectrum disorders compared to the control mice (Group 1)—this control ensures that the poly (I:C) administration did cause the expected behavioural symptoms in the maternal mouse offspring. Any effect of treatment on the behavioural symptoms of autistic spectrum disorders would be identified by differences between Group 2 and Group 3.

### Graphical Design and Statistical Analysis

All graphs were generated on graphpad prism software (version 5). Data were analysed using IBM SPSS Statistic 22.0 (EEUU). Data distribution was analysed using the Kolmogorov-Smirnov normality test. Data comparing vehicle group versus the MRX010 group were analysed using one-way ANOVA and Fisher's least significant difference (LSD) post hoc test. If ANOVA did not reveal a significant effect of treatment, a priori pairwise comparisons test against the control group was conducted. Non-normally distributed data were analysed by the Kruskal-Wallis and non-parametric Mann-Whitney U test. P<0.05 was the criterion for statistical significance.

### EXAMPLE 1b

### Assessment of Stereotyped Behaviours—the Marble Burying Test

### Rationale

This test assesses for repetitive, compulsive and anxious behaviour. A higher number of marbles buried is indicative of greater anxious or stereotyped behaviours. Indeed, Mice treated with pharmacological agents such as anxiolytics show decreased marble burying behaviour, compared to the control mice.

### Methods

Mice were individually placed into a novel polypropylene cage (35×28×18.5 cm, L×W×H), containing standard rodent (hard wood) bedding (5 cm) and 20 marbles on top of it (five rows of marbles regularly spaced 2 cm away from the walls and 2 cm apart). Experiments were conducted under a light intensity of 1000 lux. 30 minutes later, mice were removed from these cages and the number of marbles buried for more than 2/3rds of their surface was scored.

### Results

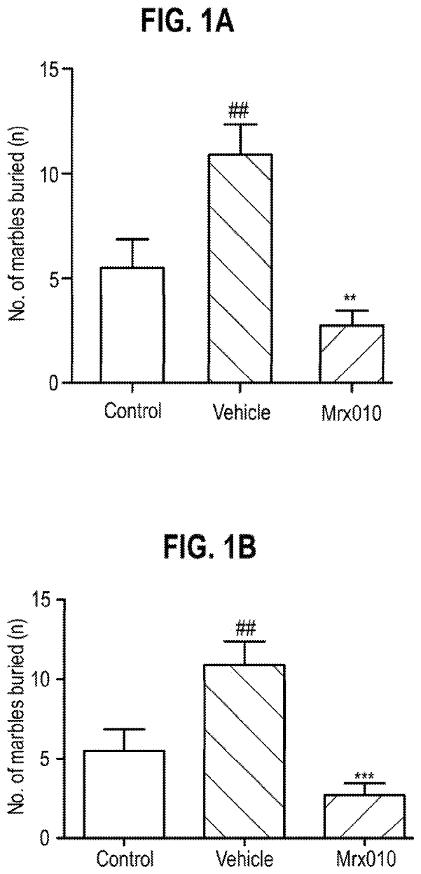

Student's t-test analysis between the control group and the vehicle MIA group revealed that the vehicle MIA mice buried more marbles compared to the control group (t(19)=3.00, P=0.007; FIG. 1A). ANOVA of the number of marbles buried revealed an effect of treatment [F(3,42)=6.37, P=0.001]. Post-hoc tests revealed that chronic treatment with Mrx0010 decreased the number of marbles buried (p<0.01; FIG. 1A). A priori pairwise comparisons revealed that MIA mice treated with Mrx010 buried less marbles than MIA vehicle mice (p<0.001; FIG. 1B).

### Conclusions

The vehicle MIA group showed significantly more marbled buried than the control group, indicating that the MIA model successfully triggered autistic spectrum disorder-like symptoms in the mice. Chronic treatment with MRX010 reduces repetitive, compulsive and anxious behaviour in MIA mice.

### EXAMPLE 1c

### Assessment of Social Behaviours—Social Transmission of Food Preference

### Rationale

Social transmission food preference is a relevant test of olfactory memory that is used in mice to assess social behaviour. In this test, observer mice interact with a demonstrator mouse that has recently eaten novel food. When observer mice are presented with a choice between the food eaten by the demonstrator and some other novel food, observer mice should prefer the food eaten by the demonstrator. Reduced food preference would indicate reduced sociability.

### Methods

This test was performed as previously described ([67]). Briefly, 18 hours prior to testing, mice were deprived of food, whereas water was available ad libitum. Food choices consisted of either 1% ground cinnamon or 2% powdered cocoa made with grounded mouse chow. A demonstrator mouse was randomly selected from each cage and the tail was marked using a blue marker to enable identification during subsequent social interactions. Demonstrator food containers were weighed before and after the 1 hour sampling sessions. A minimum of 0.2 g of consumed food was required for inclusion in the test. Demonstrator mice were placed back into their respective home cages for a 30 minute interaction period with cage-mates. Subsequently, cage-mates were individually tested for preference of cued food or novel food. Containers were weighed immediately before and after each choice session. Observed mice were then placed back into their respective home cages and the choice session was repeated 24 hours later. The test mice should smell the cinnamon or cocoa off the demonstrator mouse as a social cue, and preferentially choose the same food when given a choice between the two.

### Results

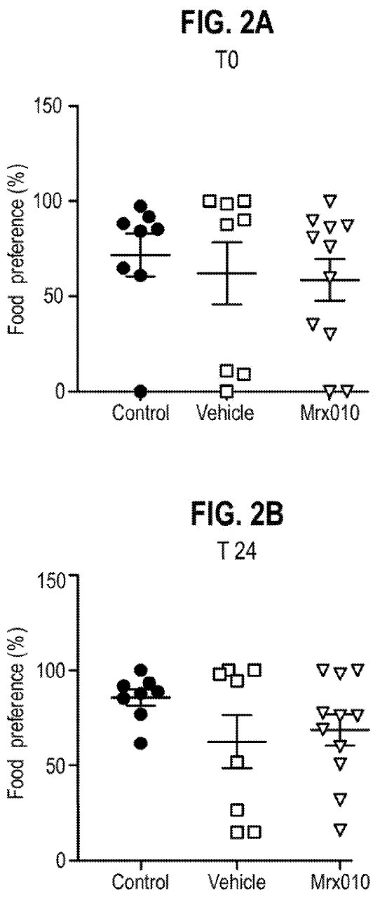

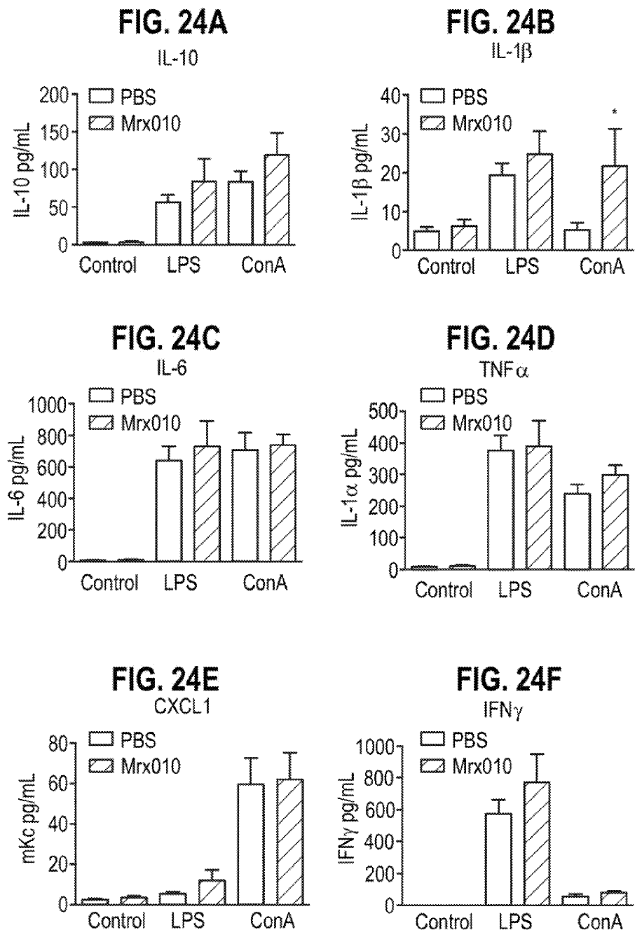

ANOVA of demonstrator cued food preference revealed no significant difference when observers were exposed to food choice immediately after demonstrator interaction (T0) (F(3,34)=0.38, P=0.77; FIG. 2A) or 24 hrs later (F(3,34)=0.85, P=0.48; FIG. 2B), irrespective of vehicle or MRX010 administration.

### Conclusions

The vehicle MIA group did not display reduced social transmission food preference (the MIA vehicle displayed no alteration in food preference compared to the control), suggesting the MIA model has not triggered the reduced sociability phenotype. Accordingly, it is not possible to determine the effects of chronic treatment with MRX010 on sociability using the MIA mouse model.

### EXAMPLE 1d

### Assessment of Depression-Like Behaviours—the Forced Swimming Test

### Rationale

The forced swim test (FST) is the most widely used experimental paradigm to assess antidepressant activity ([68]). In this test, mice are forced to swim for 6 min and the behavioural parameter scored is immobility during the last 4 min of the 6-min test. Naive animals will display escape behaviour in the form of swimming, climbing and diving before adapting an immobile floating posture. The duration of immobility is indicative of behavioural despair. Antidepressant drugs decrease the time spent immobile in this test.

### Methods

Mice are forced to swim for 6 min in a glass cylinder (24×21 cm) filled with 23-25° C. tap water to a depth of 17 cm. The FST was videotaped from a ceiling camera. The behavioural parameter scored is immobility during the last 4 min of the 6-min test.

### Results

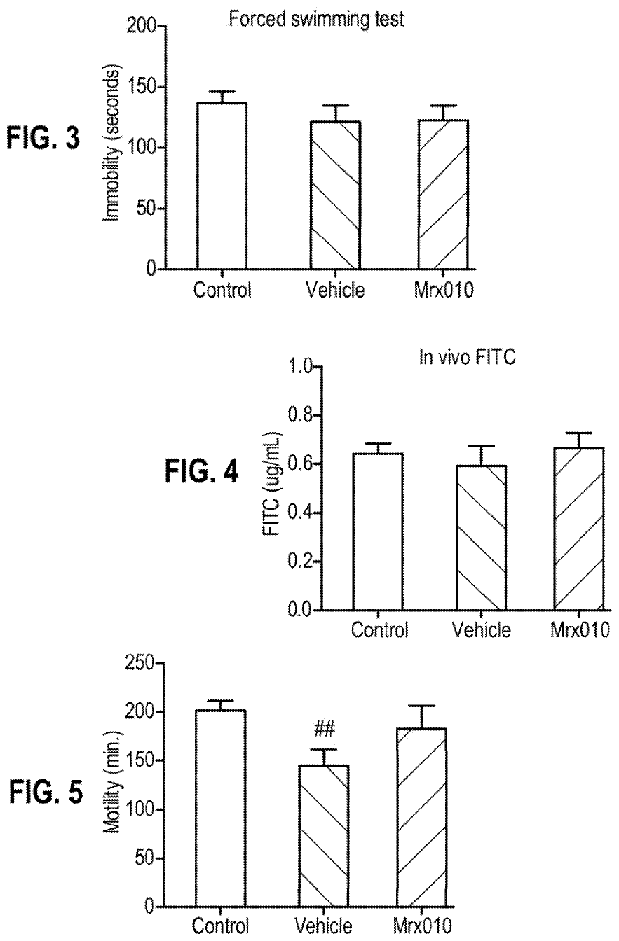

Student's t test analysis revealed no significant differences on immobility time between the control group and vehicle MIA group (t=0.8968 df=20; 0.3805). ANOVA of immobility time did not reveal an effect of treatment with MRX010 [F(3,42)=1.803; P=0.1625; FIG. 3].

### Conclusions

The vehicle MIA group did not display increased immobility time in the forced swim test (the MIA vehicle displayed no alteration in immobility time compared to the control), suggesting the MIA model has not increased depressive-like symptoms. Accordingly, it is not possible to determine the effects of chronic treatment with MRX010 on depressive-like behaviour using the MIA mouse model.

### EXAMPLE 1e

### In Vivo Intestinal Permeability Assay

### Rationale

The MIA model has been reported to lead to changes in gut barrier function. Therefore, it was important to ascertain whether chronic treatment with the biotherapeutic affects intestinal permeability.

### Methods

Test mice were single caged and food was removed overnight. Next day (at around 9 am) mice were administered by oral gavage with FITC dextran (Fluroscein-isothiocynate; MW: 4 kDa, Sigma; concentration: 600 mg/kg per animal of 80 mg/ml FITC in PBS (pH7.4)). Two hours following FITC administration, 100 μl of blood sample, from tail bleeds, was collected in heparin-coated capillary tubes and transferred to amber eppendorf and placed on ice. Samples were centrifuged 3500×g for 15 minutes, plasma was aspirated and samples were stored at −80° D for long storage.

Undiluted plasma was used to quantify FITC concentration. 25 μl of FITC was pipetted in duplicated in 384 well plate (Greiner bio one). FITC was measured with a Victor spectrometer between the ranges of 490 nm-520 nm. For a standard curve, a serial dilution of FITC was prepared in PBS (pH7.4).

In addition, after the cull of the mice in week 9, ex vivo measurements of FITC in the ileum and colon are performed.

### Results

Student's t test analysis revealed no differences between the control group and the MIA vehicle group (t(20)=0.56, P=0.58; FIG. 4). ANOVA of FITC concentrations did not reveal a significant effect of treatment [F(3,39)=2.23, P=0.08].

### Conclusions

In this experiment, the vehicle MIA group did not display altered intestinal permeability (the MIA vehicle displayed no alteration in permeability compared to the control). Furthermore, chronic treatment with MRX010 did not affect intestinal permeability in MIA mice.

### EXAMPLE 1f

### In Vivo Intestinal Motility Assay

### Rationale

The MIA model has been reported to lead to changes in gut barrier function. Therefore, it was important to ascertain whether chronic treatment with the biotherapeutic alters intestinal motility.

### Methods

Mice are singly housed prior to the commencement of the test. Mice were orally gavaged with a non-absorbable, coloured dye (Carmine Red). The time to excretion of the first coloured faecal bolus was recorded and used as an index of peristaltic motility of the whole intestine.

### Results

Student's t test analysis revealed that the vehicle MIA group exhibited increased intestinal motility (red pellet detected in less time) when compared to the control group (t19)=3.00, P=0.007). ANOVA of motility time revealed no effect of treatment [F(3,38)=0.74, P=0.54; FIG. 5].

### Conclusions

In this experiment, the vehicle MIA group displayed increased intestinal motility compared to the control. Chronic treatment with MRX010 did not affect intestinal motility compared to the control.

### Discussion of Results From the MIA Mouse Model

Chronic treatment with MRX010 was able to reverse the phenotype observed in the marble burying test in MIA mice. Chronic treatment with MRX0010 was able to reduce the number of marbles buried suggesting a reduction in stereotyped-like behaviour. No significant differences between all the groups were observed in the social transmission food test suggesting no directly observable effects in social behaviour in this model. Similarly, no significant effects of MIA protocol or live biotherapeutic treatment were observed in the forced swimming test suggesting no observable effects in depression-like behaviour in this model. The live biotherapeutic tested did not affect intestinal motility or permeability. Therefore, the MIA model has proven useful for assessing stereotyped-like, repetitive and anxious behaviour, but it did not recreate a number of other symptoms associated with autistic spectrum disorders. Nevertheless, the results display that chronic treatment with MRX010 may have a positive impact on the symptoms of autistic spectrum disorders.

### EXAMPLE 2

### The BTBR Mouse Model

The BTBR mouse model uses genetically modified, inbred mice, without any genetic modifications, that display a robust autistic-like phenotype. BTBR mice typically display stereotyped behaviours (shown by the grooming and marble burying tests); deficits in social communication (shown by the 3 chamber social interaction, resident intruder and social transmission of food preference tests); cognitive deficits (shown by the novel object recognition test); gastrointestinal abnormalities (shown by increased gut permeability of FITC); and reduced circulating oxytocin levels in the plasma. Deficits in social behaviours, increased repetitive behaviours and increased anxiety-related behaviours have been reported in this strain ([27]). Due to this robust behavioural phenotype, the BTBR mouse is an ideal animal model to assess the efficacy of novel therapeutic agents for the treatment of autistic-related behaviours. Alleviation of such symptoms by a live biotherapeutic can also be indicative of efficacy of the biotherapeutic in the treatment of other psychiatric or neurological diseases.

### EXAMPLE 2a

### Materials and Methods for BTBR Mouse Model

### Mice

Male BTBR mice were bred in house. The animals were housed in a temperature- and humidity-controlled room on a 12 hr dark cycle (lights on from 7:00-19:00 hr). All experiments were conducted in accordance with the European Directive 2010/63/EEC, the requirements of S.I. No 543 of 2012, and approved by the Animal Experimentation Ethics Committee of University College Cork.

### Strain

MRX010: Enterococcus faecium, bacteria deposited under accession number NCIMB 42487.

Biotherapeutic was provided in glycerol stock. Microbiological growth media (YCFA) was used for the culture of the agent. Live biotherapeutics were grown in the facility in anaerobic conditions.

### Live Biotherapeutic Administration

Dosing with MRX010 or vehicle commenced when the mice were 8 weeks old. These mice were treated once daily with MRX010 or phosphate buffer solution (PBS) for 3 weeks before the beginning of the behavioural battery. Mice were further treated once daily during the behavioural battery. MRX010 (1×107 to 1×109 CFU oral administration) was dissolved in PBS prior to administration.

### Administration Schedule

The treatment groups for the study are shown below. The vehicle for oral administration is PBS. Daily oral administration occurs via oral gavage.

| Group | Treatment | Number |
|---|---|---|
| 1 | Control (PBS, oral gavage) | 10 |
| 2 | MRX010 (oral gavage in PBS) | 10 |

### Fecal Collection

Fresh fecal samples were collected from individual mice every week until the end of the study. At least 20 mg of fresh faeces were placed in a microcentrifuge tube, place immediately on ice and then stored at −80° C.

### Experimental Design and Methods

As outlined above, dosing with MRX010 commenced when the mice were 8 weeks old. The initial dosing took place for 3 weeks before the behavioural experiments encompassing tests of sociability, anxiety, stereopathy and cognition. The behavioural battery occurred in the following order: marble burying test at week 4; the elevated plus maze at week 5; the open field and novel object recognition tests, and the social transmission of food preference tests at week 6; the female urine sniffing and social interaction tests at week 7, and the forced swimming test at week 9. The carmine red gastrointestinal motility assay and gastrointestinal permeability assay tail bleeds occurred during weeks 8 and 9 respectively. Finally, in weeks 10 to 11, the mice were killed for splenocyte stimulation and ex vivo measurement of FITC in the ileum and colon.

The effects of live biotherapeutic treatment in the BTBR model on stereotyped, social and depression-like behaviours, along with gastrointestinal parameters (permeability and motility) are outlined in the following examples.

Group 1, listed in the table above, represents the control BTBR mice, which would be expected to show phenotypes associated with autistic spectrum disorders. Any effect of treatment on the behavioural symptoms of autistic spectrum disorders would be identified by differences between Group 1 and Group 2.

### Graphical Design and Statistical Analysis

All graphs were generated on graphpad prism software (version 5). Data were analysed using IBM SPSS Statistic 22.0 (EEUU). Data distribution was analysed using the Kolmogorov-Smirnov normality test. Data comparing vehicle group versus the MRX010 group were analysed using one-way ANOVA and Fisher's least significant difference (LSD) post hoc test. If ANOVA did not reveal a significant effect of treatment, a priori pairwise comparisons test against the control group was conducted. Non-normally distributed data were analysed by the Kruskal-Wallis and non-parametric Mann-Whitney U test. P<0.05 was the criterion for statistical significance.

### EXAMPLE 2b

### Assessment of Social Behaviours—Social Transmission of Food Preference

### Rationale

Social transmission food preference is a relevant test of olfactory memory that is used in mice to assess social behaviour. In this test, observer mice interact with a demonstrator mouse that has recently eaten novel food. When observer mice are presented with a choice between the food eaten by the demonstrator and some other novel food, observer mice should prefer the food eaten by the demonstrator. Reduced food preference would indicate reduced sociability.

### Methods

This test was performed as previously described ([67]). Briefly, 18 hours prior to testing, mice were deprived of food, whereas water was available ad libitum. Food choices consisted of either 1% ground cinnamon or 2% powdered cocoa made with grounded mouse chow. A demonstrator mouse was randomly selected from each cage and the tail was marked using a blue marker to enable identification during subsequent social interactions. Demonstrator food containers were weighed before and after the 1 hour sampling sessions. A minimum of 0.2 g of consumed food was required for inclusion in the test. Demonstrator mice were placed back into their respective home cages for a 30 minute interaction period with cage-mates. Subsequently, cage-mates were individually tested for preference of cued food or novel food. Containers were weighed immediately before and after each choice session. Observed mice were then placed back into their respective home cages and the choice session was repeated 24 hours later. The test mice should smell the cinnamon or cocoa off the demonstrator mouse as a social cue, and preferentially choose the same food when given a choice between the two.

### Results

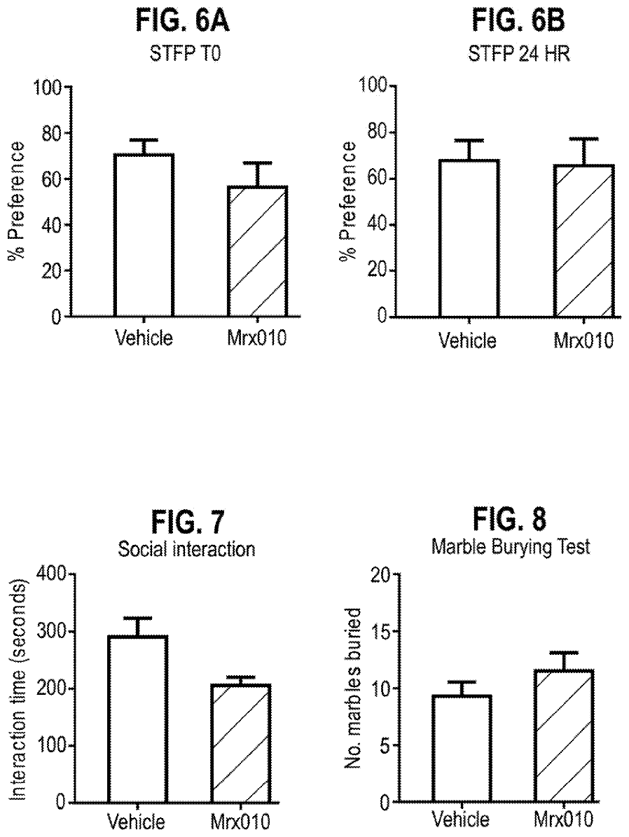

ANOVA of demonstrator cued food preference revealed no significant difference when observers were exposed to food choice immediately after demonstrator interaction (T0) (F(3,36)=1.123; P=0.354; FIG. 6A) or 24 hrs later (F(3,38)=0.138; P=0.936; FIG. 6B).

### Conclusions

Treatment with MRX010 did not affect the sociability of BTBR mice in the social transmission food preference test.

### EXAMPLE 2c

### Assessment of Social Behaviours—Forced Intruder Test

### Rationale

This procedure evaluates social interaction behaviour between rodents. By placing an intruder mouse into the resident mouse's home-cage, one can assess social interaction and aggressive behaviour.

### Methods

Each session consisted of placing an intruder mouse into a resident mouse's home-cage for a period of 10 minutes. Experiments were videotaped using a ceiling camera to allow for measuring several behavioural parameters. The amount of time that the animals spent interacting was recorded.

### Results

ANOVA of interaction time did not reveal an effect of treatment [F(3,36)=1.905; P=0.1462; FIG. 7].

### Conclusions

Treatment with Mrx010 did not influence social behaviour of BTBR mice in the social interaction test.

### EXAMPLE 2d

### Assessment of Stereotyped Behaviours—the Marble Burying Test

### Rationale

This test assesses for repetitive, compulsive and anxious behaviour. A higher number of marbles buried is indicative of greater anxious or stereotyped behaviours. Indeed, Mice treated with pharmacological agents such as anxiolytics show decreased marble burying behaviour, compared to the control mice.

### Methods

Mice were individually placed into a novel polypropylene cage (35×28×18.5 cm, L×W×H), containing standard rodent (hard wood) bedding (5 cm) and 20 marbles on top of it (five rows of marbles regularly spaced 2 cm away from the walls and 2 cm apart). Experiments were conducted under a light intensity of 1000 lux. 30 minutes later, mice were removed from these cages and the number of marbles buried for more than ⅔rds of their surface was scored.

### Results

ANOVA of the number of marbles buried did not reveal an effect of treatment [F(3,39)=0.835; P=0.483; FIG. 8], suggesting that treatment with Mrx010 did not affect stereotyped behaviour in BTBR mice.

### Conclusions

Chronic treatment with MRX010 did not affect repetitive, compulsive and anxious behaviour in BTBR mice.

### EXAMPLE 2e

### Assessment of Anxiety-Like Behaviour—the Elevated Plus Maze

### Rationale

The elevated plus maze (EPM) is a widely used test to assess anxiety-like behaviours in rodents. The EPM assesses general anxiety behaviour, with less anxious mice spending more time in the open arms of the maze. An increase in open arm activity (duration) reflects anti-anxiety behaviour.

### Methods

The set up consisted of a grey plastic cross-shaped maze 1 meter elevated from the floor, comprising two open (aversive) and two closed (safe) arms (50×5×15 cm walls). Experiments occurred under red light (7 lux). Mice were placed into the centre of the maze facing an open arm (to avoid direct entrance into a closed arm) and were allowed to explore the arena for a duration of five minutes. Experiments were videotaped using a ceiling camera to allow for measuring several behavioural parameters. The percentage of time spent and the number of entries in each arm was measured for anxiety-like behaviour and locomotor activity, respectfully. Entrance into an arm was defined as all four paws inside the arm.

### Results

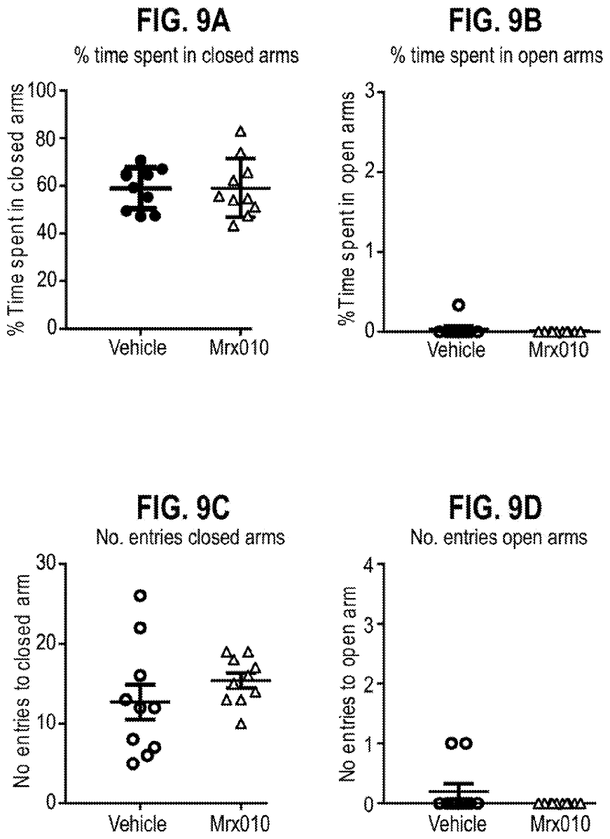

ANOVA of percentage time spent in closed arms revealed no effect of treatment [F(3,39)=0.556; P=0.647; FIG. 9A]. Kruskal Wallis non-parametric analysis of percentage time spent in open arms [Chi squared: 10.831; df=3; P=0.013; FIG. 9B] followed by non-parametric Mann-Whitney U test revealed that mice treated with MRX010 spent no more time in the open arms compared to the vehicle group. ANOVA of the number of entries into the closed arms revealed no effect of treatment [F(3,39)=0.556; P=0.647; FIG. 9C]. Kruskal Wallis non-parametric analysis of number of the entries into the open arms [chi-squared=10.315; df=3; P=0.016; FIG. 9D] followed by non-parametric Mann-Whitney U test revealed no effect of treatment on the number of entries in the open arms.

### Conclusions

Chronic treatment with MRX010 did not affect anxiety-like behaviour in the elevated plus maze in BTBR mice.

### EXAMPLE 2f

### Assessment of Anxiety-Like Behaviour—the Open Field Arena

### Rationale

The open field arena is used to assess the response of exposure to a novel stressful environment and locomotor activity. Naïve mice naturally spend most of their time alongside the walls of the arena, as it is less exposed than the centre of the arena. An increase in duration of time spent in the centre represents a decrease in anxiety-like behaviour.

### Methods

Mice were individually placed into an open field arena (43×35×25, L×w×h) and allowed to explore for 10 minutes. Experiments occurred under a light intensity of 60 lux. Experiments were videotaped using a ceiling camera to allow for measuring several behavioural parameters using Ethovision software. The distance travelled was scored for locomotor activity.

### Results

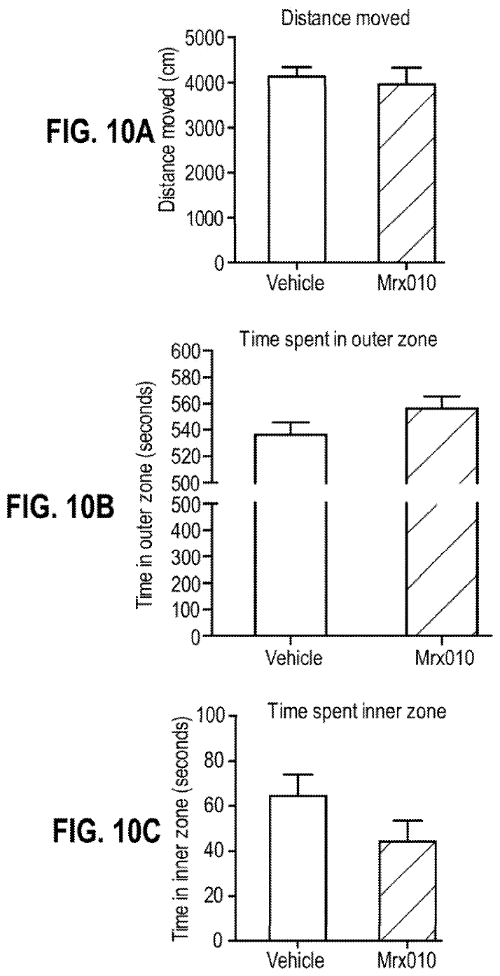

ANOVA of distance moved did not reveal an effect of treatment upon locomotor activity in the open field arena [F(3,37)=1.325; P=0.282, FIG. 10A]. ANOVA of time spent in the outer zone did not reveal an effect of treatment [F(3,37)=1.598; P=0.208; FIG. 10B]. ANOVA of time spent in the inner zone did reveal an effect of treatment [F(3,36)=3.636; P=0.023; FIG. 10C].

### Conclusions

Treatment with Mrx010 had no effect upon locomotor activity and anxiety-like behaviour in the open field arena in BTBR mice.

### EXAMPLE 2g

### Assessment of Depression-Like Behaviour—the Forced Swimming Test

### Rationale

The forced swim test (FST) is the most widely used experimental paradigm to assess antidepressant activity. Naïve animals will display escape behaviour in the form of swimming, climbing and diving before adapting an immobile floating posture. The duration of immobility is indicative of behavioural despair. Antidepressant drugs decrease the time spent immobile in this test.

### Methods

Mice are forced to swim for 6 min in a glass cylinder (24×21 cm) filled with 23-25° C. tap water to a depth of 17 cm. The FST was videotaped from a ceiling camera. The behavioural parameter scored is immobility during the last 4 min of the 6-min test.

### Results

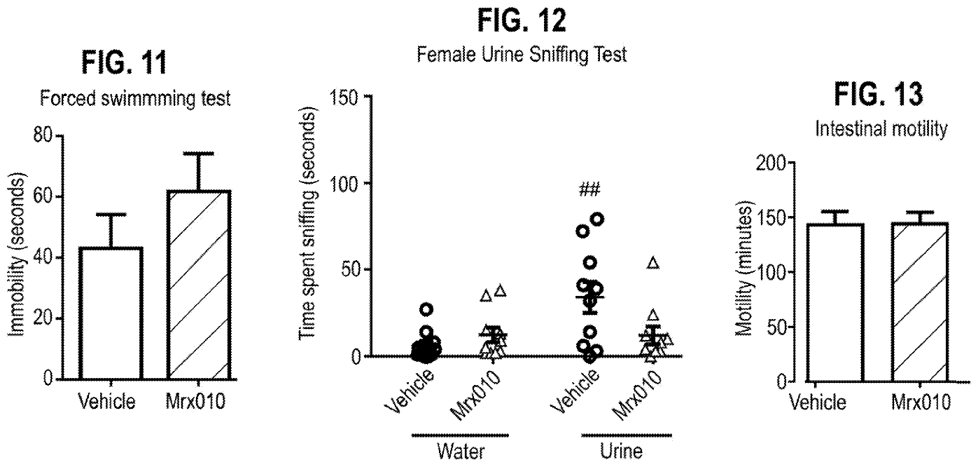

ANOVA of immobility time did not reveal an effect of treatment on immobility time of BTBR mice in the FST [F(3,38)=1.879; P=0.151; FIG. 11].

### Conclusions

Treatment with MRX010 did not influence immobility time of BTBR mice in the forced swimming test.

### EXAMPLE 2h

### Assessment of Depression-Like Behaviour—the Female Urine Sniffing Test

### Rationale

The female urine sniffing test (FUST) is used to assess anhedonic-like behaviour in rodents. A reduction in sniffing time suggests social avoidance/anhedonia while an increase represents an increase in social behaviour/hedonic behaviour.

### Methods

Experimental mice are singly housed one week prior to the test. During the test, a cotton tip applicator, dipped in sterile water, is placed into the home cage and mice are allowed to sniff/investigate for three minutes. Following this three minute test, the cotton tip applicator is removed. 45 minutes later, a new cotton tip applicator is dipped into female urine (collected from female mice of the same strain that are in the estrous stage of their cycle), and placed into the cage. Mice are allowed to sniff/investigate this for a further three minutes. The amount of time spent sniffing the water and urine is recorded.

### Results

For the vehicle group, Mann-Whitney U test revealed a significant increase in the time spent sniffing urine relative to the time spent sniffing water [t=2.976 df=18; P=0.0081]. For exposure to water, Kruskal Wallis non-parametric analysis of time spent sniffing did not reveal an effect of treatment in the water group [Chi squared: 6.352;df=3; P=0.096]. For exposure to urine, Kruskal Wallis non-parametric analysis of time spent sniffing did not reveal an effect of treatment [Chi squared: 3.639; df=3; P=0.303, FIG. 12].

### Conclusions

Treatment with MRX010 had no effect upon the time spent sniffing urine in BTBR mice.

### EXAMPLE 2i

### In Vivo Gastrointestinal Motility Assay

### Rationale

This procedure is used to assess in vivo intestinal motility.

### Methods

Mice are singly housed prior to the commencement of the test. Mice were orally gavaged with a non-absorbable, coloured dye (Carmine Red). The time to excretion of the first coloured faecal bolus was recorded and used as an index of peristaltic motility of the whole intestine.

### Results

ANOVA of motility time revealed no effect of treatment [F(3,39)=2.072; P=0.121; FIG. 13].

### Conclusions

Treatment with MRX010 did not influence intestinal motility.

### EXAMPLE 2j

### Organ Weight and Colon Length

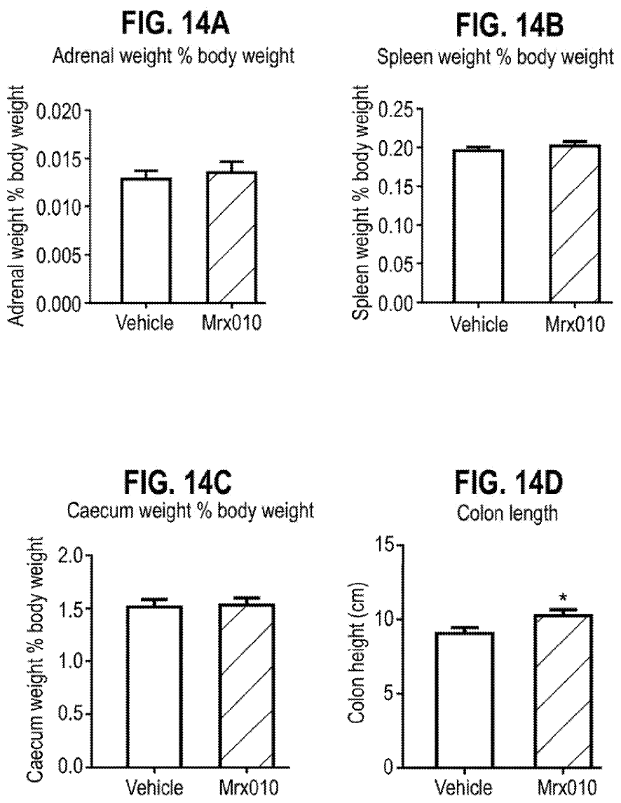

ANOVA of organ weight as a percentage of body weight did not reveal an effect of treatment for the adrenals [F(3,37)=0.208; P=0.890; FIG. 14A}, spleen F(3,35)=0.629; P=0.601; FIG. 14B] or caecum [F(3,37)=0.883; P=0.460; FIG. 14C]. ANOVA of colon length revealed an effect of treatment [F(3,37)=5.635; P=0.003; FIG. 14D]. Post-hoc analysis revealed that chronic treatment with MRX010 (p<0.05 relative to the vehicle group) increased colon length in BTBR mice.

### Discussion of Results From BTBR Mouse Model

Overall, treatment with MRX010 did not have significant effects in tests for stereotyped behaviour, social behaviour, anxious behaviour and depression-related behaviours in BTBR mice. With the exception of colon length, the live biotherapeutic assessed in the current study did not affect the several physiological parameters measured (adrenal weight, spleen weight, caecum weight and carmine red). Treatment with MRX010 increased colon length when compared to vehicle treated mice, however, no changes in intestinal motility were detected in carmine red test.

### Overall Conclusions Regarding MRX010 in the Treatment of Autistic Spectrum Disorders

MRX010 was shown to be effective in the treatment of stereotyped, repetitive and anxious behaviour in the MIA mice model. Therapies that reverse behavioural and biological phenotypes in mouse models of autism are expected to be effective against human disease. The resolution of behavioural phenotypes might not be expected in all animal models of autism. It is possible that the symptoms exhibited by the BTBR were not severe enough for any beneficial effect of MRX010 to be measurable.

The EMA Guidelines on the clinical development of medicinal products for the treatment of autism spectrum disorder state that, due to the heterogeneity of the diseases, it may not be possible to achieve a significant effect on all core symptoms with a single compound, and so short term efficacy has to be demonstrated on at least one core symptom. The MRX010 live biotherapeutic has shown effective treatment of at least one core symptom or autistic spectrum disorder, so it and related E. faceium strains are expected to be effective against human disease.

### EXAMPLE 3

### Stability Testing

A composition described herein containing at least one bacterial strain described herein is stored in a sealed container at 25° C. or 4° C. and the container is placed in an atmosphere having 30%, 40%, 50%, 60%, 70%, 75%, 80%, 90% or 95% relative humidity. After 1 month, 2 months, 3 months, 6 months, 1 year, 1.5 years, 2 years, 2.5 years or 3 years, at least 50%, 60%, 70%, 80% or 90% of the bacterial strain shall remain as measured in colony forming units determined by standard protocols.

### EXAMPLE 4

### Assessing the Effects of Subchronic Treatment With MRX010 Upon Central and Peripheral Oxytocin Levels in C57BL/6 Mice

The bacterial strains were prepared and administered as outlined in Examples 1 and 2 above. The C57BL/6 mice were treated with live biotherapeutic for six days in 7 experimental treatment groups each with 10-12 mice. Subsequently, the hypothalamus was dissected from the mice and the levels of oxytocin in the hypothalamus were detected by radioimmunoassay (RIA), In addition, levels of oxytocin in the plasma were detected by RIA. Furthermore, the levels of oxytocin receptors, interleukins and other inflammatory markers, and vasopressin hormones were detected by RIA and other analytical methods.

### EXAMPLE 5

### Maternal Immune Activation (MIA) Mouse Model

### EXAMPLE 5a

### Materials and Methods for MIA Mouse Model Mice

Female C57/B16 mice (8 weeks old) and age matched males were purchased from Harlan UK. After 1-week habituation these animals were mated. At embryonic day 12.5, females received either an injection of the viral mimetic poly-IC to activate the maternal immune system, or a saline vehicle injection. The male offspring from these animals were separated from their mothers at 3 weeks old.

All experiments were conducted in accordance with the European Directive 2010/63/EEC, the requirements of the S.I No 543 of 2012, and approved by the Animal Experimentation Ethics Committee of University College Cork.

### Strain

MRX010: Enterococcus faecium, bacteria deposited under accession number NCIMB 42487. Live biotherapeutics were grown in the facility in anaerobic conditions.

### Live Biotherapeutic Administration

For dosing, male offspring received daily oral gavage of PBS or the live biotherapeutic MRX010 prepared to 10⁹ cfu/mL in PBS. Daily administration of the live biotherapeutic or control commenced at 8 weeks of age (3 weeks before the beginning of the behavioural battery). Dosing continued daily throughout the behavioural paradigm.

### Administration Schedule

The vehicle for oral administration is PBS. Daily oral administration occurs via oral gavage.

### EXAMPLE 5b

### Assessment of Stereotyped Behaviours—the Self-Grooming Test

### Rationale

This test assesses for repetitive, compulsive and anxious behaviour. A longer duration spent grooming suggests an increased stereotype behaviour; or higher levels of anxiety in response to a new environment.

### Methods

Self-grooming is evaluated in a 6.5 cm diameter×10 cm tall, clear glass beaker covered with a filter top. Experimental animals will be brought to the test room to habituate up to 1 hour prior to the test. The test is approximately 20 minutes in duration. The investigator records the cumulative time spent by the test animal grooming. Between tests the beakers are cleaned thoroughly for next use.

### Results

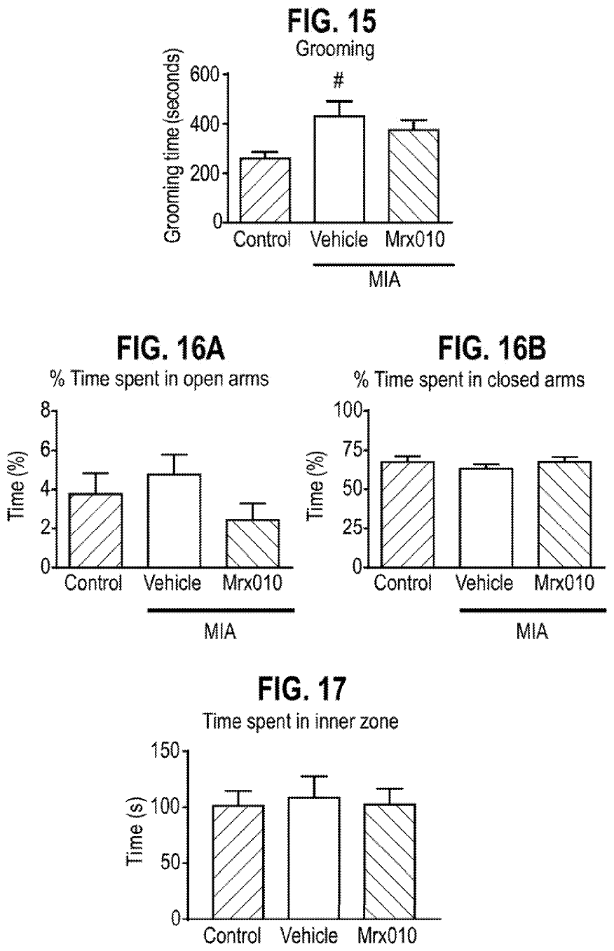

Student's t-test analysis between the control group and the vehicle MIA group revealed that the vehicle MIA mice displayed an increased duration of grooming compared to the control group (t(18)=2.628, P=0.017; FIG. 15). Treatment with MRX010 attenuated the increase in grooming behaviour (t(18)=0.767, p=0.453).

### Conclusions

### MRX010 Appeared to Reduce the Stereotyped/Neophobic Behaviour of MIA Mice in the Self-Grooming Test.

### EXAMPLE 5c

### Assessment of Anxiety-Like Behaviours—the Elevated Plus Maze

### Rationale

The elevated plus maze (EPM) is a widely used test to assess anxiety-like behaviours in rodents. The EPM assesses general anxiety behaviour, with less anxious mice spending more time in the open arms of the maze. An increase in time or number of entries into an open arm is an index of reduced anxiety.

### Methods

The set up was made of a grey plastic cross-shaped maze 1 m elevated from the floor, comprising two open (fearful) and two closed (safe) arms (50×5×15 cm walls or 1 cm no wall). Experiments occurred under red light (˜5 lux). Mice were individually placed into the center of the maze facing an open arm (to avoid direct entrance into a closed one) and were allowed 5-min free exploration. Experiments were videotaped using a ceiling camera for further parameters analysis using Ethovision software (3.1 version, Noldus, TrackSys, Nottingham, UK). The percentage of time spent, distance moved and the number of entries in each arm were measured, for anxiety behaviour and locomotor activity, respectively (entrance in an arm was defined as all four paws inside the arm).

### Results

No significant increase in anxiety-like behaviour in MIA mice was observed in the elevated plus maze. In particular, the vehicle MIA mice did not display more time in the closed arms (t18=2.628, p=0.017) and less time on the open arms (t18=2.628, p=0.017) (indicative of an increase in anxiety-like behaviour). Chronic treatment with MRX010 did not observably affect the anxiety-like behaviour as there was no change in the duration on the open arms (t18=2.628, p=0.017) compared to the closed arms (t18=2.628, p=0.017) (see FIGS. 16A and B).

### Conclusions

MRX010 appeared not to affect the anxiety of MIA mice in the elevated plus maze. As the vehicle MIA group did not display increased anxiety-like behaviour, it appears that the MIA model did not trigger an increased anxiety phenotype. Accordingly, it is not possible to determine the effects of chronic treatment with MRX010 on anxiety in the elevated plus maze using the MIA mouse model.

### EXAMPLE 5d

### Assessment of Anxiety-Like Behaviours—the Open Field Arena

### Rationale

The open field arena is used to assess the response of exposure to a novel stressful environment and locomotor activity. Naïve mice naturally spend most of their time alongside the walls of the arena, as it is less exposed than the centre of the arena. Mice naturally like to stick to the sides of an arena, so the longer they spend in the centre is an index of a reduction in anxiety-like behaviour.

### Methods

To assess the response to a novel stressful environment and locomotor activity, mice were placed into open arena (40×32×23 cm, L×w×h) with ˜60 lux lighting and allowed to explore for 10-mins. Experiments were videotaped using a ceiling camera for further parameter analysis using Ethovision software (3.1 version, Noldus, TrackSys, Nottingham, UK). The distance travelled and the latency to enter a virtual central zone (defined at 50% away from the edges) was scored.

### Results

There was no change in anxiety like behaviour in MIA mice in the open field arena. In particular, there was no reduction in the time spent in the inner zone (an indicator of increased anxiety) compared to the control (t(19)=3.061, p=0.763). In addition, MRX010 did not observably affect the duration of time spent in the inner zone (t(17)=0.253, p=0.803) (FIG. 17).

### Conclusions

MRX010 appeared not to affect the anxiety of MIA mice in the open field arena. As the vehicle MIA group did not display increased anxiety-like behaviour, it appears that the MIA model did not trigger an increased anxiety phenotype. Accordingly, it is not possible to determine the effects of chronic treatment with MRX010 on anxiety in the open field arena using the MIA mouse model.

### EXAMPLE 5e

### Assessment of Social Behaviours—Female Urine Sniffing Test

### Rationale

The female urine sniffing test (FUST) is used to assess anhedonic-like behaviour in rodents. An increase in time spent sniffing the urine odour suggests an increased interest in social behaviour.

### Methods

On the morning of the test, vaginal smears from 20 C57BL/6J female mice were taken and analyzed for the cycle stage of the animal. Only urine from mice in estrus was collected for the test. Male mice were transferred to a quiet, dimly lit room prior to the test, and habituated to an empty cotton tip applicator inserted into their homecage. One hour later a cotton tip dipped in sterile water was presented to the animal for three minutes and sniffing time was measured. After a 45-min intertrial interval, during which mice were left undisturbed, presentation of a cotton tip infused with 60 μl of fresh urine from a female mouse in estrus was carried out for three minutes and sniffing duration was timed.

### Results

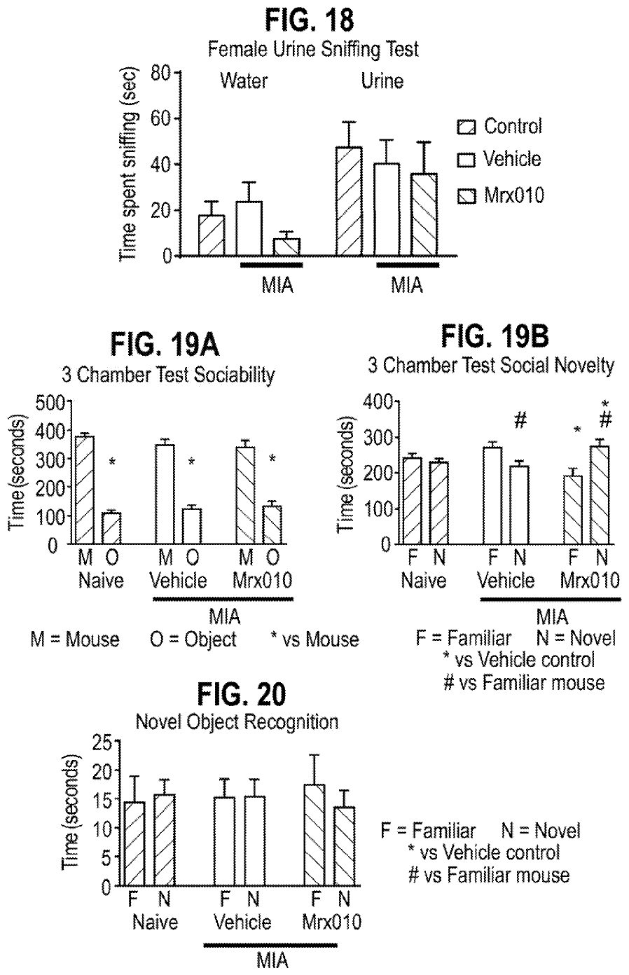

Mice in all groups spent more time sniffing urine (as a social cue) compared to water (as a neutral cue) (F(1,57)=9.971, p=0.003). There was no deficit in this social response in MIA mice (F(1,39)=0.002, p=0.959), nor was there any observable effect of Mrx010 in this animal model (F(1,36)=0.364, p=0.550) (FIG. 18).

### Conclusions

The vehicle MIA group did not display a significant reduction in urine sniffing, and so the MIA model did not trigger a reduced level of social behaviour. Accordingly, it is not possible to determine the effects of chronic treatment with MRX010 on social behaviour in the female urine sniffing test using the MIA mouse model.

### EXAMPLE 5f

### Assessment of Social Behaviours—Three-Chambered Social Approach Task (Three-Chamber Test)

### Rationale

The 3-Chamber Social Interaction Test (3-CSIT) is a well validated ethologically relevant model that assesses social interaction between sex-matched conspecifics and allows for readouts of social novelty and social preference in mice. The test allows mice to freely explore between an inanimate object or sex-matched conspecific mice. Some animal models, as used here, have deficits in this social paradigm; and live biotherapeutics were investigated for whether they could ameliorate these deficits.

### Methods

The social testing apparatus was a rectangle, three-chambered box. Each chamber was 20 cm L×40 cm W×22 cm H. Dividing walls were made with small circular openings (5 cm in diameter) allowing access into each chamber. Two identical wire cup-like cages, with a bottom diameter of 10 cm, 13 cm in height and bars spaced 1.2 cm, allowing nose contact between the bars, but prevented fighting, were placed inside each side chamber in bilaterally symmetric positions. The test has three phases of 10 min each: 1) habitation 2) mouse versus object 3) novel mouse versus familiar mouse. Experiments were videotaped using a ceiling camera for further parameters analysis using Ethovision software (3.1 version, Noldus, TrackSys, Nottingham, UK). For the first phase the test mouse was placed into the middle chamber and allowed to explore the entire box with empty small wire cages inside for a 10-min habituation session. After the habituation period, the test mouse is removed from the testing box for a short interval while an object is placed in one side chamber and an unfamiliar conspecific male mouse (no prior contact with the test subject) in the other side chamber, both enclosed in a wire cup-like cage. During phase two, the test mouse is placed in the middle chamber and allowed to explore the entire box for 10 min. The amount of time spent exploring the object or mouse in each chamber and the number of entries into each chamber were evaluated. The location of the unfamiliar mouse in the left vs right side chamber was systematically alternated between trials. An entry was defined as all four paws in one chamber. During the third phase an object was replaced with an unfamiliar mouse serving as a novel mouse and in the other chamber the mouse used in phase two was kept the same, now serving as familiar mouse. After every trial, all chambers and cup-like wire cages were cleaned with 10% ethanol, dried and ventilated for a few minutes to prevent olfactory cue bias and to ensure proper disinfection. Lack of innate side preference was confirmed during the initial 10 min of habituation to the entire arena. Control animals naturally are more interested in a conspecific mouse more than an inanimate object (sociability). In a similar vein, control animals spend more time interacting with a novel unfamiliar mouse then one they have already had interactions with.

### Results

There was a clear preference for test animals to spend more time interacting with a conspecific mouse than an inanimate object (F(1,46)=261.1, p<0.001) (FIG. 19A). In the MIA mice there was no effect on sociability as the MIA mice preferred to interact with the mouse over the object to the same extent as the naïve control (F(1,32)=1.984, p=0.169). In line with the lack of effect on sociability, there was no observable effect of MRX010 on sociability (F(1,32)=0.1905, p=0.665). In the test for social novelty (i.e. interaction with a familiar or novel mouse), there was no overall difference between the time spent exploring novel and familiar mice (F(1,48)=0.206, p=0.652). However, the vehicle MIA mice tended to prefer interaction with a familiar rather than a novel mouse (F(2,48)=8.825, p=0.005) (FIG. 19B). MRX010 reversed this deficit in social behaviour (social novelty or recognition) observed in the vehicle MIA group (F(1,34)=13.44, p=0.001), displaying increased time interacting with the novel mouse compared to the familiar mouse.

### Conclusions

The MIA model did not demonstrate an effect on sociability (object compared to mouse interaction) and so it is not possible to comment on the impact of MRX010 treatment in this test. However, the MIA model did affect social novelty interaction. Importantly, MRX010 reversed the deficit of social novelty interaction, and therefore, chronic treatment with MRX010 increases sociability of MIA mice in the three-chamber test.

### EXAMPLE 5g

### Assessment of Cognitive Performance—Novel Object Recognition Test Rationale

This test is used to test recognition memory and assess cognitive performance. Control animals will discriminate between an object they have had time to explore and a new object. Some animal models have deficits in their ability to recognise these novel objects. Live biotherapeutics were investigated for their ability to ameliorate these deficits in cognitive performance.

### Methods

Mice were placed in the middle of a grey plastic rectangular box (40×32×23 cm, L×W×H) under a dimly light, 60 lux at the level of the arena, for 10 min. 24 h after mice were placed in the box with the two identical objects for a total time of 10 min (acquisition phase). After a 24 h, one of the two identical objects were substituted with a novel object and mice were placed in the middle of the box at the mid-point of the wall opposite the sample objects for a total time of 10 min (retention phase). Animals were acclimatized to the testing room for 30 min prior each experiment. Box and objects were cleaned with alcohol 10% to avoid any cue smell between each trial. Experiments were videotaped using a ceiling camera for further parameter analysis. Directed contacts with the objects, include any contact with mouth, nose or paw or minimal defined distance were counted as an interaction.

### Results

There was no discrimination between familiar and novel objects in any of the groups tested (F(2,60)=0.273, p=0.762). There was no deficit in cognitive performance in MIA mice (F(1,40)=0.028, p=0.867), nor was there any observable effect of MRX010 on cognitive performance when discriminating between a familiar and a novel object (F(1,38)=0.291, p=0.593) (FIG. 20).

### Conclusions

The MIA mice displayed no defects in cognitive performance behaviour and so this model did not trigger the expected deficits in cognition. Therefore, it is not possible to determine the effects of chronic MRX010 treatment on cognitive performance of MIA mice in the novel object recognition test.

### Discussion of Results From MIA Mouse Model

MRX010 ameliorated the increase in grooming/stereotyped behaviour in MIA mice. In the three-chamber test for social novelty/discrimination, MIA mice did not discriminate between a novel mouse and a familiar mouse, demonstrating reduced sociability and recognition. However, chronic treatment with MRX010 reversed this deficit in social behaviour.

Taken together with the data disclosed above (Example 1) these data suggest effects for MRX010 in the amelioration of deficits in stereotypical, anxiety-like and social behaviour in an environmental animal model of autism.

### EXAMPLE 6

### Ex Vivo Preclinical Study

In this preclinical study the effect of 6 day administration of the live biotherapeutic MRX010 on a number of readouts including gut permeability and function, metabolic profile (SCFAs), systemic immune activation (splenocyte assay), plasma levels of amino acids, central neurotransmitter release and gene expression for inflammatory, endocrine and neurotransmitter markers in the hippocampus, amygdala and prefrontal cortex was assessed.

### EXAMPLE 6a

### Materials and Methods for Preclinical Study

### Mice

BALBc (Envigo, UK) adult male mice were group housed under a 12 h light-dark cycle; standard rodent chow and water were available ad libitum. All experiments were performed in accordance with European guidelines following approval by University College Cork Animal Ethics Experimentation Committee. Animals were 8 weeks old at the start of the experiment.

### Strain

MRX010: Enterococcus faecium, bacteria deposited under accession number NCIMB 42487. Live biotherapeutics were grown in the facility in anaerobic conditions.

### Study Design and Live Biotherapeutic Administration

Mice were allowed to habituate to their holding room for one week after arrival into the animal unit. They receive oral gavage (2000 μL, dose) of live biotherapeutics at a dose of 1×10⁹ CFU for 6 consecutive days between 15:00 and 17:00. On day 7, the animals are decapitated and tissues are harvested for experimentation.

MRX010 administration at this concentration for this dosing regime does not negatively impact on systemic and central physiological events. From a translational perspective, these data suggest that this live biotherapeutic may have a high tolerability profile with minimal non-desirable side-effects.

### Tissue Collection

Animals were sacrificed in a random fashion regarding treatment and testing condition; sampling occurred between 9.00 a.m. and 2:30 p.m. Trunk blood was collected in potassium EDTA (Ethylene Diamine Tetra Acetic Acid) tubes and spun for 15 min at 4000 g. Plasma was isolated and stored at −80° C. for further analysis. The brain was quickly excised, dissected and each brain region was snap-frozen on dry ice and stored at −80° C. for further analysis. Spleen was removed, collected in 5 mL RPMI media (with L-glutamine and sodium bicarbonate, R8758 Sigma +10% FBS (F7524, Sigma)+1% Pen/Strep (P4333, Sigma)) and processed immediately after culls for ex-vivo immune stimulation. Intestinal tissue (2 cm segments of ileum and colon closest to the caecum were excised, and the furthest 1 cm of tissue from the caecum were used) were mounted into the Ussing chambers for intestinal permeability assay. A further 1 cm of ileum and colon tissue was taken for tight junction gene expression analysis. The caecum was removed, weighed and stored at −80° C. for SCFAs analysis.

### Intestinal Permeability

Mice were euthanized by cervical dislocation, and the distal ileum and colon were removed, placed in chilled Krebs solution opened along the mesenteric line and carefully rinsed. Preparations were then placed in Ussing chambers (Harvard Apparatus, Kent, UK, exposed area of 0.12 cm²) as described previously (Hyland and Cox, 2005) with oxygenated (95% O₂, 5% CO₂) Krebs buffer maintained at 37° C. 4 kDa FITC-dextran was added to the mucosal chamber at a final concentration of 2.5 mg/mL; 200 μL samples were collected from the serosal chamber every 30 min for the following 3 h.

### Short Chain Fatty Acids Analysis in the Caecal Content

Caecum content was mixed and vortexed with MilliQ water and incubated at room temperature for 10 min. Supernatants were obtained by centrifugation (10000 g, 5 min, 4° C.) to pellet bacteria and other solids and filtration by 0.2 μm. It was transferred to a clear GC vial and 2-Ethylbutyric acid (Sigma) was used as the internal standard. The concentration of SCFA was analyzed using a Varian 3500 GC flame-ionization system, fitted with a with a ZB-FFAP column (30 m×0.32 mm×0.25 mm; Phenomenex). A standard curve was built with different concentrations of a standard mix containing acetate, propionate, iso-butyrate, n-butyrate, isovalerate and valerate (Sigma). Peaks were integrated by using the Varian Star Chromatography Workstation version 6.0 software. All SCFA data are expressed as μmol/g.

### Plasma Levels of Amino Acids

Animals were sacrificed in a random fashion regarding treatment and testing condition; sampling occurred between 9.00 a.m. and 2:30 p.m. Trunk blood was collected in potassium EDTA (Ethylene

Diamine Tetra Acetic Acid) tubes and spun for 15 min at 4000 g. Plasma was isolated and stored at −80° C. for further analysis. Plasma was diluted with 0.2 mol/L sodium citrate buffer, pH 2.2 to yield 250 nmol of each amino acid residue. Samples were diluted with the internal standard norleucine, to igve a final concentration of 125 nm/mL. Amino acids were quantified using a Jeol JLC-500/V amino acid analyser (Jeol Ltd, Garden City, Herts, UK) fitted with a Jeol Na+ high performance cation exchange column.

### Spleen Cytokine Assay

Spleens were collected immediately in 5 mL RPMI media following sacrifice and cultured immediately. Spleen cells were first homogenised in the RPMI media. The homogenate step was followed by RBC lysis step where the cells were incubated for 5 mins in lml of RBC lysis buffer (11814389001 ROCHE, Sigma). 10 ml of the media was added to stop the lysis and followed by 200g centrifugation for 5 mins. This was followed by final step where the cells were passed through 40 um strainer. The homogenate was then filtered over a 40 um strainer, centrifuged at 200 g for 5 min and resuspended in media. Cells were counted and seeded (4,000,000/mL media). After 2.5 h of adaptation, cells were stimulated with lipopolysaccharide (LPS-2 μg/ml) or concanavalin A (ConA-2.5 μg/ml) for 24 h. Following stimulation, the supernatants were harvested to assess the cytokine release using Proinflammatory Panel 1 (mouse) V-PLEX Kit (Meso Scale Discovery, Md., USA) for TNFα, IL-10, IL-1β, Interferon γ, CXCL2 and IL6. The analyses were performed using MESO QuickPlex SQ 120, SECTOR Imager 2400, SECTOR Imager 6000, SECTOR S 600.

### Monoamine Analysis

Neurotransmitter concentration was analysed by HPLC on samples from the brainstem. Briefly, brainstem tissue was sonicated in 500 μl of chilled mobile phase spiked with 4 ng/40 μl of N-Methyl 5-HT (Sigma Chemical Co., UK) as internal standard. The mobile phase contained 0.1 M citric acid, 5.6 mM octane-1-sulphonic acid (Sigma), 0.1 M sodium dihydrogen phosphate, 0.01 mM EDTA (Alkem/Reagecon, Cork) and 9% (v/v) methanol (Alkem/Reagecon), and was adjusted to pH 2.8 using 4 N sodium hydroxide (Alkem/Reagecon). Homogenates were then centrifuged for 15 min at 22,000×g at 4° C. and 40 μl of the supernatant injected onto the HPLC system which consisted of a SCL 10-Avp system controller, LECD 7 electrochemical detector (Shimadzu), a LC-10AS pump, a CTO-10A oven, a SIL-10A autoinjector (with sample cooler maintained at 40 C) and an online Gastorr Degasser (ISS, UK). A reverse-phase column (Kinetex 2.6 u C18 100×4.6 mm, Phenomenex) maintained at 30° C. was employed in the separation (Flow rate 0.9 ml/min). The glassy carbon working electrode combined with an Ag/AgCl reference electrode (Shimdazu) operated a +0.8 V and the chromatograms generated were analyzed using Class-VP 5 software (Shimadzu). The neurotransmitters were identified by their characteristic retention times as determined by standard injections, which run at regular intervals during the sample analysis. The ratios of peak heights of analyte versus internal standard were measured and compared with standard injection. Results were expressed as ng of neurotransmitter per g fresh weight of tissue.

Central and Gastrointestinal Gene Expression Analysis

Total RNA was extracted using the mirVanaTM miRNA Isolation kit (Ambion/Llife technologies, Paisley, UK) and DNase treated (Turbo DNA-free, Ambion/life technologies) according to the manufacturers recommendations. RNA was quantified using NanoDrop™ spectrophotometer (Thermo Fisher Scientific Inc., Wilmington, Del., USA) according to the manufacturer's instructions. RNA quality was assessed using the Agilent Bioanalyzer (Agilent, Stockport, UK) according to the manufacturer's procedure and an RNA integrity number (RIN) was calculated. RNA with RIN value >7 was used for subsequent experiments. RNA was reverse transcribed to cDNA using the Applied Biosystems High Capacity cDNA kit (Applied Biosystems, Warrington, UK) according to manufacturer's instructions. Briefly, Multiscribe Reverse Transcriptase (50 U/μL) (1)(2)(1)(10) was added as part of RT master mix, incubated for 25° C. for 10 min, 37° C. for 2 h, 85° C. for 5 min and stored at 4° C. Quantitative PCR was carried out using probes (6 carboxy fluorescein—FAM) designed by Applied Biosystems to mouse specific targeted genes, while using (3-actin as an endogenous control. Amplification reactions contained 1 μl cDNA, 5 μl of the 2X PCR Master mix (Roche), 900 nM of each primer and were brought to a total of 10 μl by the addition of RNase-free water. All reactions were performed in triplicate using 96-well plates on the LightCycler®480 System. Thermal cycling conditions were as recommended by the manufacturer (Roche) for 55 cycles. To check for amplicon contamination, each run contained no template controls in triplicate for each probe used. Cycle threshold (Ct) values were recorded. Data was normalized using β-actin and transformed using the 2-ΔΔCT method and presented as a fold change vs. control group.

### Statistical Analysis

Normally distributed data are presented as mean±SEM; Non-parametric datasets are presented as median with inter-quartile range. Unpaired two-tailed t-test were applied to analyse parametric data and Mann-Whitney test was used for non-parametric. Spearman's rank correlation coefficient was employed for the correlation analysis in the pooled datasets. A p value<0.05 was deemed significant in all cases.

### EXAMPLE 6b

### Results of Preclinical Study

### Intestinal Permeability and Gastrointestinal Gene Expression

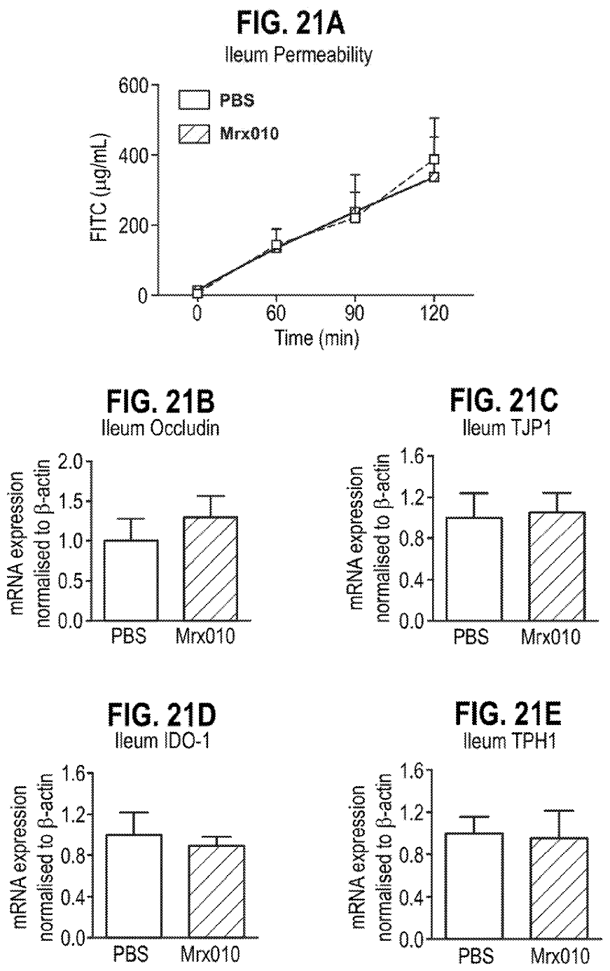

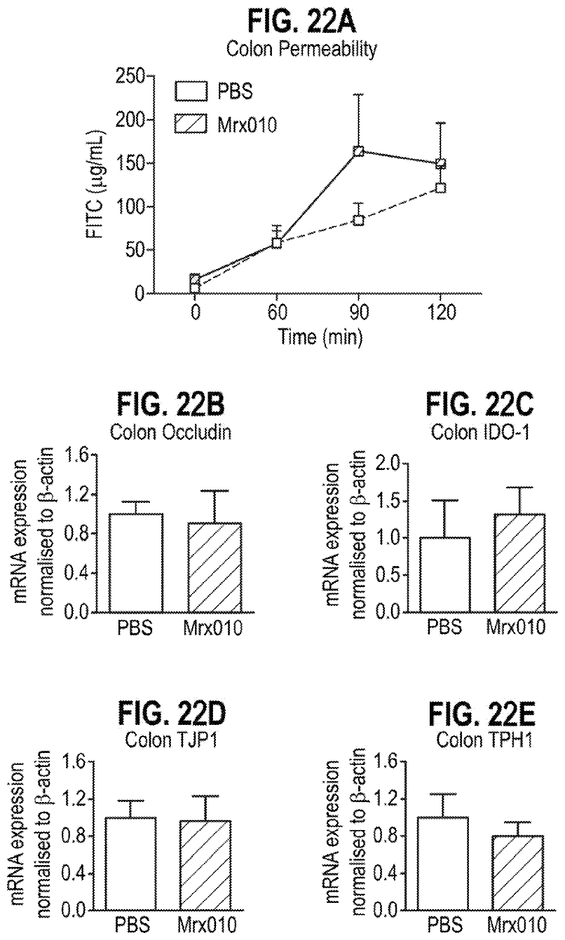

Using the passage of FITC from the luminal to the serosal side of the Ussing chamber as an index of gut permeability, it was determined that MRX010 had no observable effect on ileum (FIG. 21A (F(3,24)=0.107, p=0.96)) or colon (FIG. 22A (F(3,27)=1.141, p=0.351)) tissue permeability. Furthermore, MRX010 had no significant effect on mRNA expression of the tight junction proteins (involved in maintaining the integrity of the gut barrier) TJP1 (FIG. 21C (t(12)=0.16, p=0.876) and FIG. 22D (t(8)=0.114, p=0.912)) or occludin (FIG. 21B (t(11)=0.72, p=0.487) and FIG. 22B (t(8)=0.272, p=0.972)); the enzyme IDO-1(the first and rate-limiting enzyme in the tryptophan/kynurenine pathway) (FIG. 21D (t(12)=0.398, p=0.698) and FIG. 22C (t(8)=0.51, p=0.623)); nor TPH1 (an isoform of the enzyme tryptophan hydroxylase, responsible for the synthesis of serotonin) (FIG. 21E ((t(12)=0.157, p=0.878) or FIG. 22E ((t(9)=0.533, p=0.611)) in ileum or colon tissue.

### SCFA Expression in Caecal Content

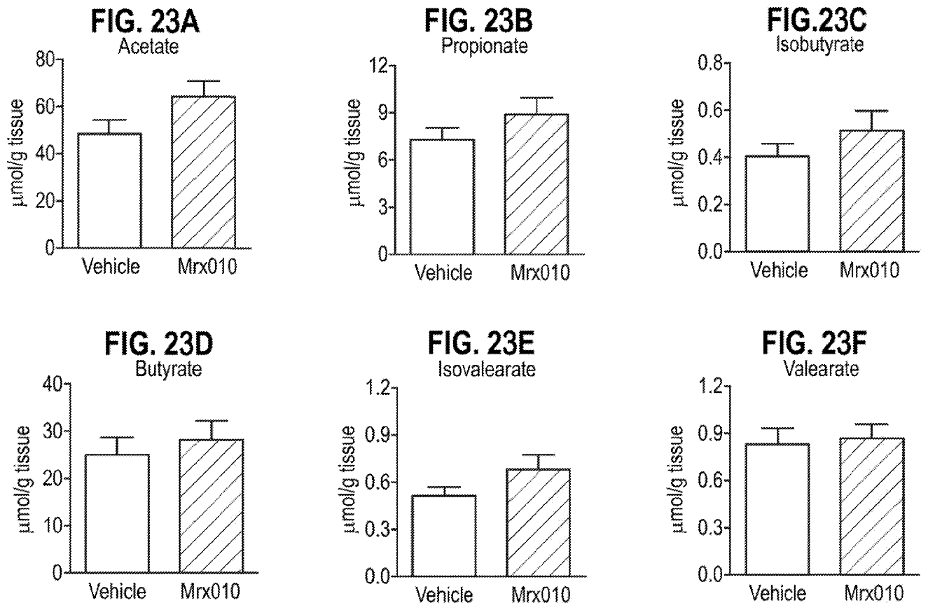

Short chain fatty acids (SCFAs) are produced when non-digestible fibres from the diet are fermented by bacteria in the gut. Chronic administration of MRX010 had no observable effect on the caecal production of the short chain fatty acids acetate (t(12)=1.787, p=0.099), proprionate (t(12)=1.29, p=0.222), isobutyrate (t(11)=1.152, p=0. 174), butyrate (t(12)=0.577, p=0.575), isovalearate (t(11)=1.584, p=0.142) or valearate (t(12)=0.27, p=0.292), when compared to vehicle PBS administration (FIG. 23A-F).

### Spleen Cytokine Assay

The ex vivo splenocyte assay involves challenging the splenocytes (cells isolated from the spleen—a main organ involved in immune defence), with a bacteriomimetic (lipopolysaccharide) or viral mimetic (concavalin A) challenge. MRX010 increased IL-1β expression in response to concavalin A compared to the PBS control, suggesting a facilitatory role in inflammation in response to viral challenge. However, MRX010 appeared not to affect splenocyte release of proinflammatory (IFNγ, TNFα) nor anti-inflammatory (IL-10, IL-6) or CXCL1 (marker of immune response activation) in response to LPS or concavalin A stimulation when compared to administration of the PBS control (FIGS. 24A-24F and the table below outlines the significant differences).

| | IL-10 | IL-Iβ | IL-6 | TNFα | CXCL1 | IFNγ |
|---|---|---|---|---|---|---|
| Control | t(12) = 0.461, | t(11) = 0.718, | t(12) = 0.394, | t(12) = 0.969, | t(12) = 0.873, | t(10) = 0.836, |
| | p = 0.653 | p = 0.488 | p = 0.701 | p = 0.352 | p = 0.400 | p = 0.423 |
| LPS | t(12) = 0.995, | t(11) = 0.830, | t(12) = 0.518, | t(12) = 0.155, | t(11) = 1.309, | t(10) = 1.12, |
| | p = 0.340 | p = 0.424 | p = 0.614 | p = 0.880 | p = 0.217 | p = 0.291 |
| ConA | t(11) = 1.117, | t(8) = 2.54, | t(12) = 0.226, | t(12) = 1.323, | t(12) = 0.123, | t(7) = 1.06, |
| | p = 0.288 | p = 0.035 * | p = 0.825 | p = 0.210 | p = 0.904 | p = 0.324 |

### Plasma Levels of Amino Acids

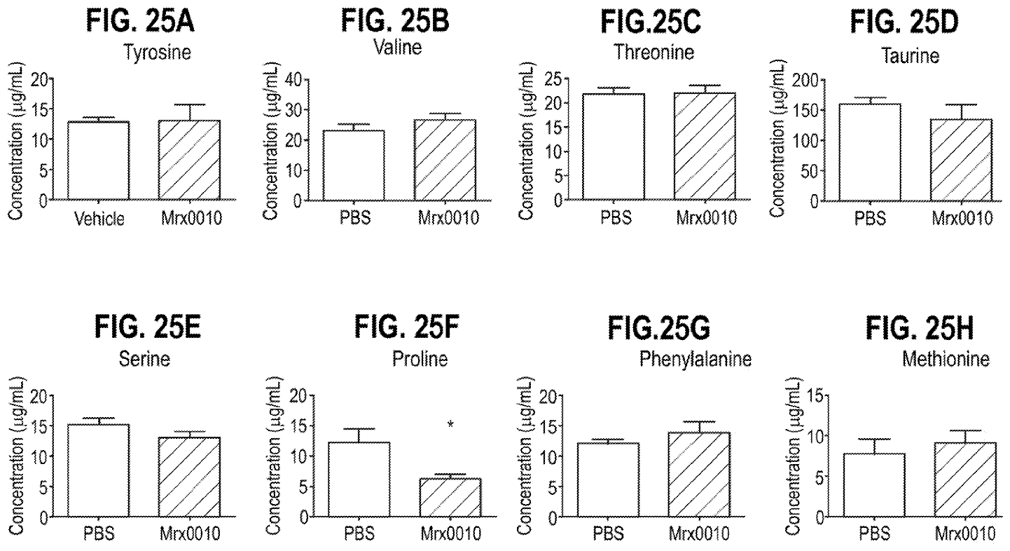

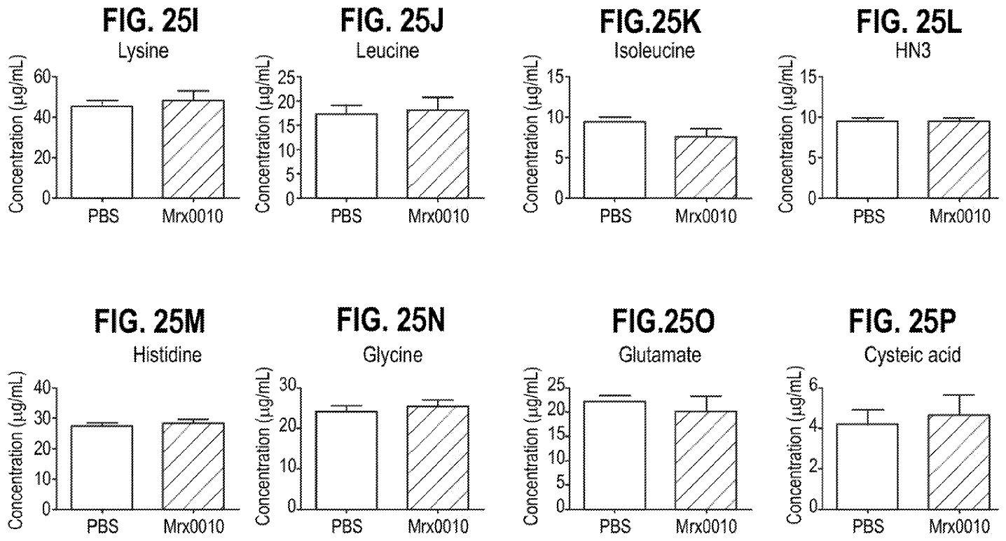

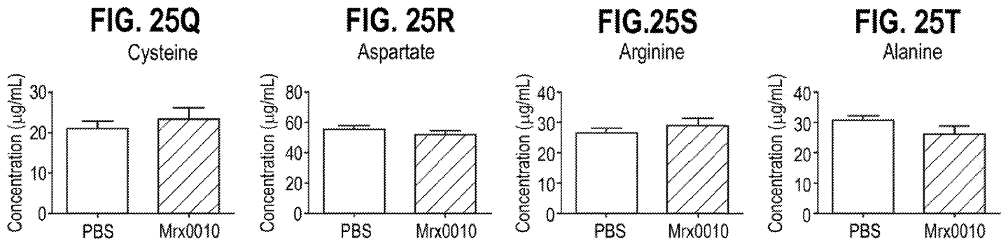

Trunk blood was collected for amino acid analysis in the plasma to give an index of the biosynthesis and catabolism of essential amino acids by changes in microbiota. MRX010 decreased proline levels in the plasma (t(9)=2.733, p=0.023), but appeared not to alter levels of tyrosine (t(12)=0.078, p=0.39), valine (t(12)=1.152, p=0.272), threonine (t(11)=0.072, p=0.944), taurine (t(12)=1.03, p=0.323), serine (t(12)=1.334, p=0.207), phenylalanine (t(12)=0.086, p=0.343), methionine (t(11)=0.564, p=0.584), lysine (t(12)=0.496, p=0.629), leucine (t(12)=0.289, p=0.778), isoleucine (t(12)=0.169, p=0.107), HN3 (t(12)=0.021, p=0.984), histidine (t(12)=0.516, p=0.615), glycine (t(12)=0.608, p=0.555), glutamate (t(12)=0.674, p=0.513), cysteic acid (t(11)=0.375, p=0.715), cysteine (t(12)=0.718, p=0.487), aspartate (t(12)=1.009, p=0.313), arginine (t(12)=0.883, p=0.395) or alanine (t(12)=4.525, p=0.153) (see FIGS. 25A-25T).

### Monoamine Changes in Brainstem

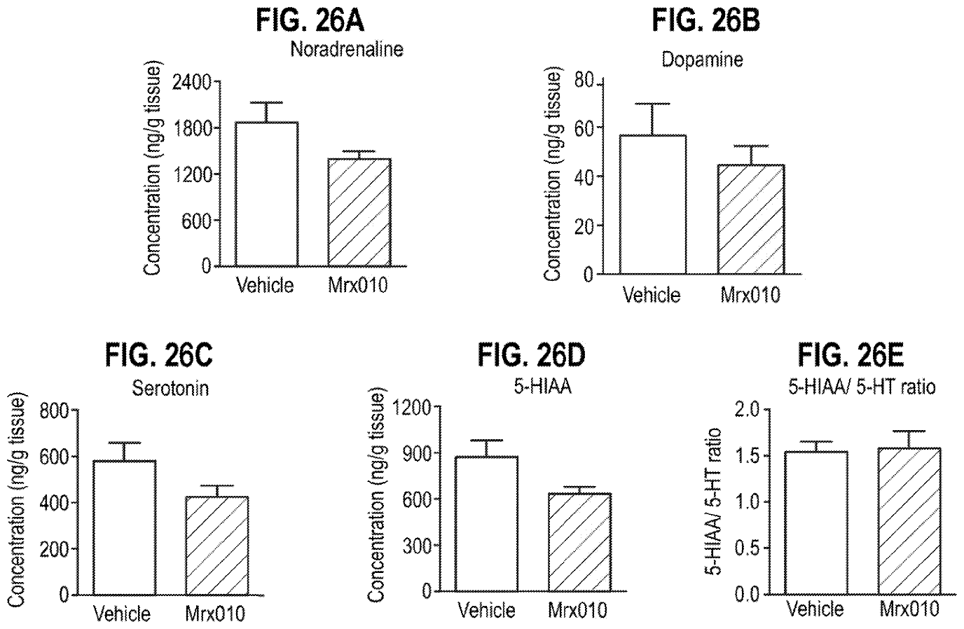

Chronic administration of MRX010 appeared not to alter levels of noradrenaline (t(12)=1.551, p=0.147), dopamine (t(12)=0.731, p=0.479), serotonin (t(12)=0.154, p=0.149), 5-HIAA (5-hydroxy-indole-acetic acid; a metabolite of 5-HT) (t(12)=1.858, p=0.088), or serotonin turnover (the ratio of 5-HIAA:5-HT) (t(12)=0.202, p=0.844) as determined by unpaired 2-tailed t-test (FIG. 26A-26E).

### Central Gene Expression

Expression of genes for neurotransmitter receptors (serotonin receptor 1a(5-HT1a), dopamine D1 receptor, GABAB receptor subunit B1, GABAA receptor, NMDA2A and NMDA2B receptor), inflammatory markers (IL-1β, IL6, CD11b, TNFα and TLR4), and endocrine markers (corticosterone releasing factor (CRF), corticosterone releasing factor receptors 1 and 2 (CRFR1, CRFR2), brain-derived neurotrophin factor (BDNF), vasopressin receptor, oxytocin receptor, glucocorticoid receptor and mineralocorticoid receptor) were analysed in brain tissue from the amygdala, prefrontal cortex and hippocampus.

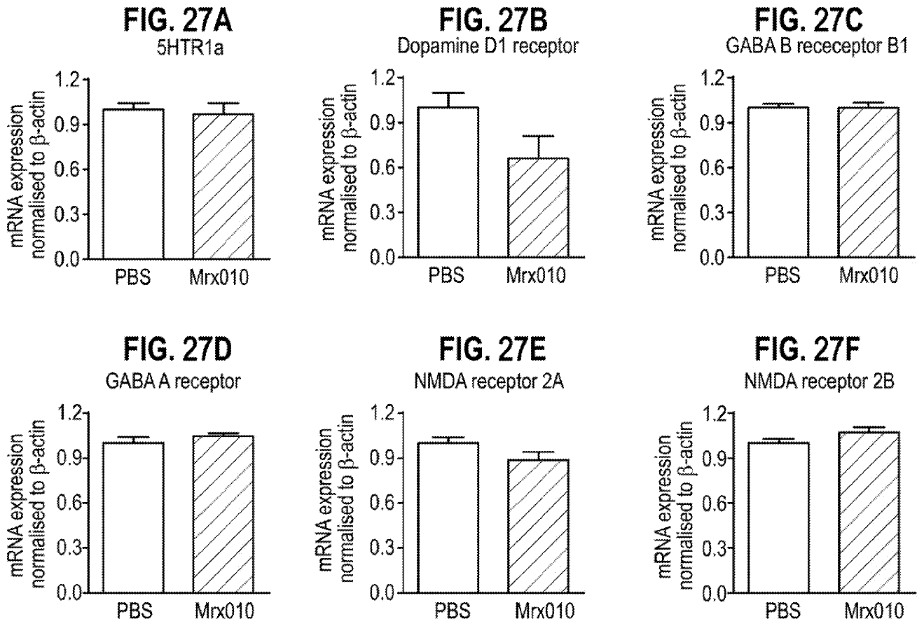

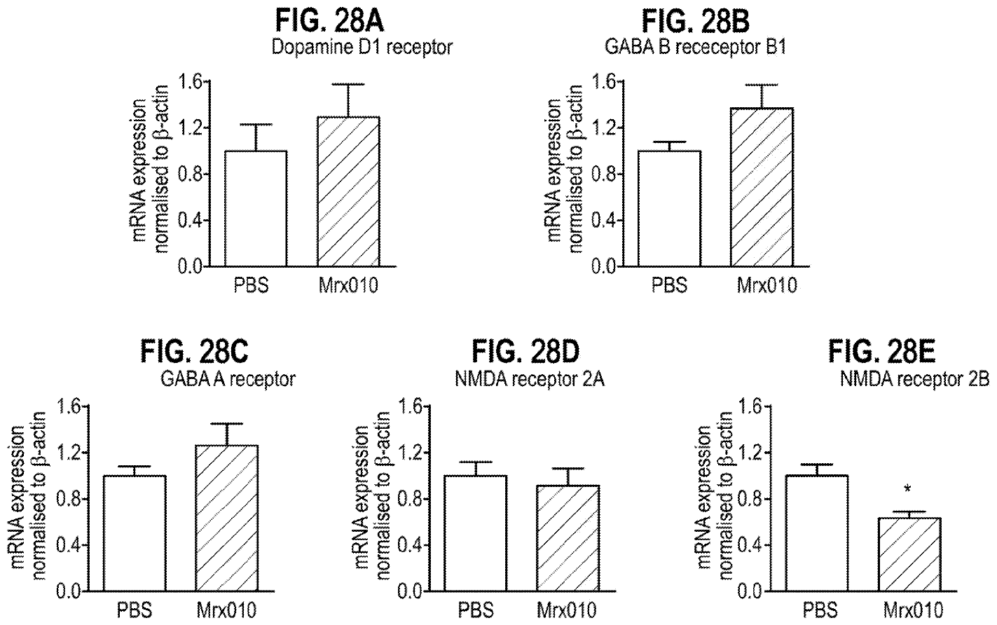

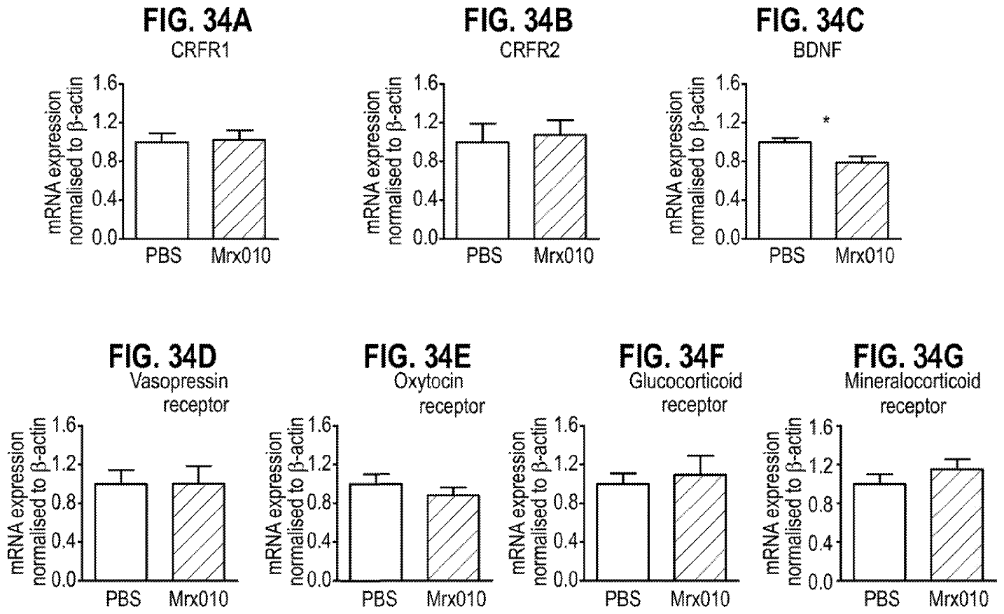

Chronic administration of MRX010 did not show an appreciable effect on mRNA expression of neurotransmitter receptors, inflammatory markers or endocrine markers in the hippocampus or prefrontal cortex (FIGS. 27A-27F, 29A-29E, 30A-30E, 32A-32C, 33A-33H and 35A-35F). In the amygdala, a limbic region of the brain associated with anxiety, fear and memory consolidation, mRNA expression of the neurotransmitter receptor, NMDA receptor 2B, and the endocrine marker, BDNF, were decreased (FIGS. 28A-28E and 34A-34G) while there was no observable effect on the other inflammatory, neurotransmitter or endocrine targets (FIGS. 28A-28E, 31A-31C and 34A-34G).

MRX010 did not show an appreciable effect on hippocampal gene expression of the neurotransmitter receptors serotonin 1a (5-HT1a) (t(11)=0.382, p=0.710), dopamine D1 receptor (t(10)=1.906, p=0.086), GABAB receptor B1 subunit (t(12)=0.029, p=0. 978), GABAA receptor (t(12)=0.926, p=0.373), NMDA receptor subunit 2A (t(11)=1.757, p=0.107), NMDA receptor subunit 2B (t(10)=1.52, p=0.159) (FIG. 27A-27F).

MRX010 did not show an appreciable effect on amygdala gene expression of the neurotransmitter receptors dopamine D1 receptor (t(12)=0.808, p=0.435), GABAB receptor B1 subunit (t(11)=1.793, p=0.100), GABAA receptor (t(11)=1.352, p=0.204), NMDA receptor subunit 2A (t(12)=0.455, p=0.658), but did decrease the expression of NMDA receptor subunit 2B (t(11)=2.737, p=0. 019) (FIG. 28A-28E).

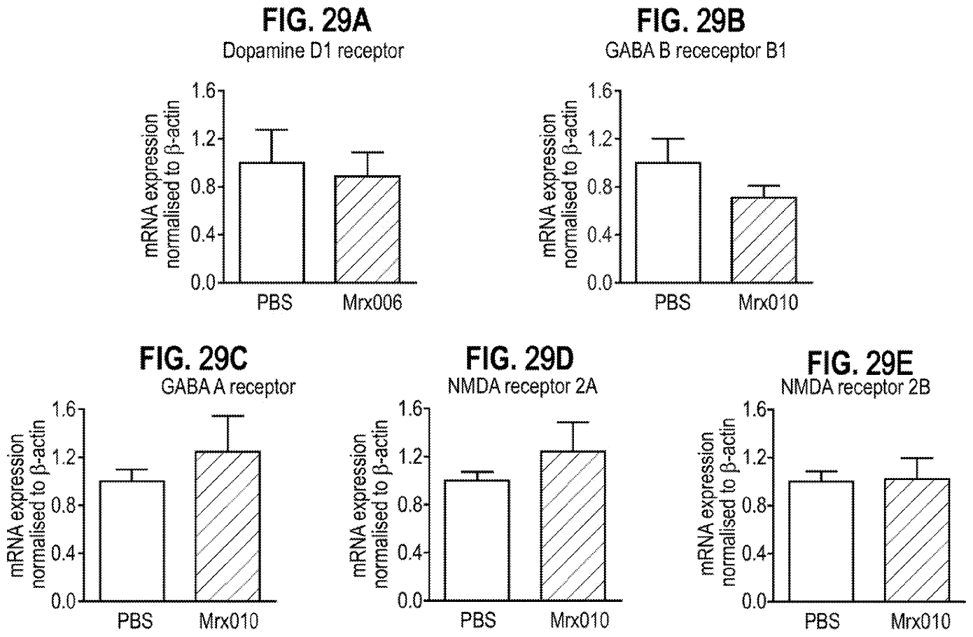

MRX010 did not show an appreciable effect on PFC gene expression of the neurotransmitter receptors dopamine D1 receptor (t(11)=0.295, p=0.773), GABAB receptor B1 subunit (t(11)=1.08, p=0.303), GABAA receptor (t(10)=0.787, p=0.450), NMDA receptor subunit 2A (t(12)=0.108, p=3.012), NMDA receptor subunit 2B (t(11)=1.213, p=0. 906) (FIG. 29A-29E).

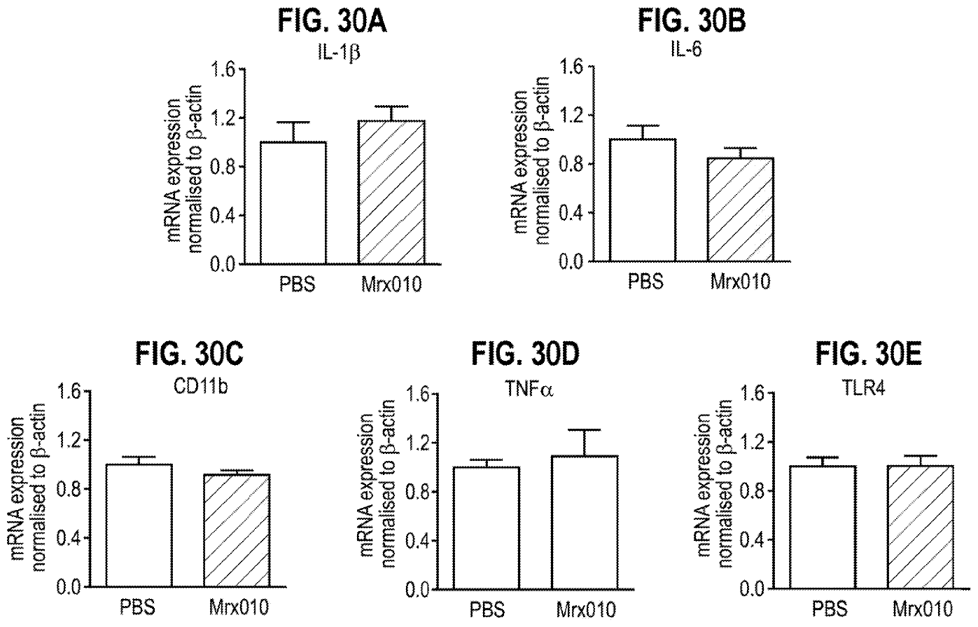

MRX010 did not show an appreciable effect on Hippocampal gene expression of the inflammatory markers IL-1β (t(10)=0.863, p=0.408), IL-6 (t(11)=0.957, p=0.359), CD11b (t(12)=1.083, p=0.300), TNFα (t(9)=0.507, p=0. 624), TLR4 (t(12)=0.025, p=0. 980) (FIG. 30A-30E).

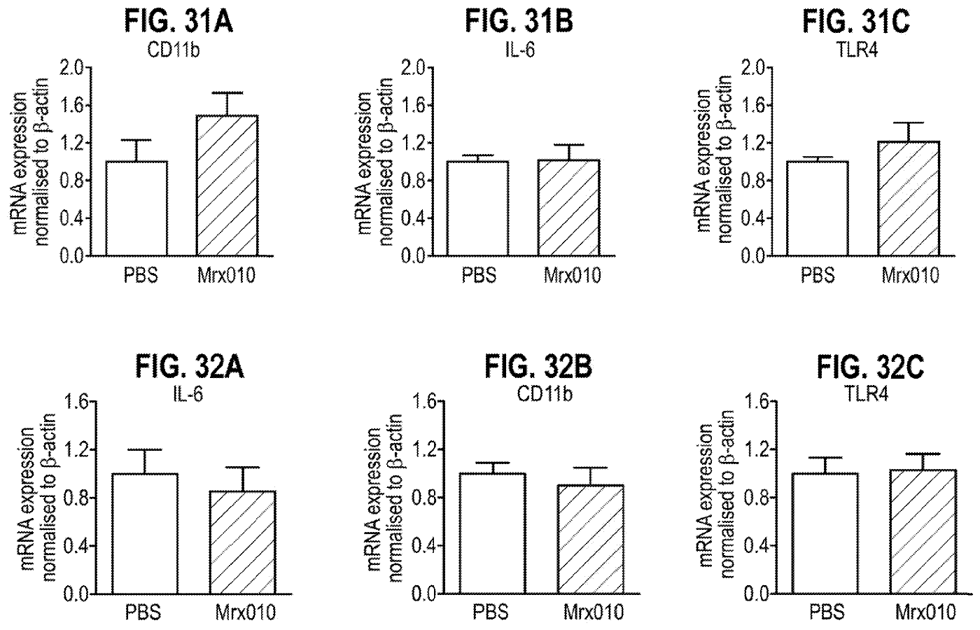

MRX010 did not show an appreciable effect on amygdalar gene expression of the inflammatory markers IL-6 (t(11)=0.980, p=0.924), CD11b (t(12)=1.438, p=0.176), TLR4 (t(11)=1.081, p=0.303) (FIG. 31A-31C).

MRX010 did not show an appreciable effect on mRNA expression of TLR4 (t(11)=0.135, p=0.895), IL-6 (t(11)=0.492, p=0.633) or CD11b (t(11)=0.608, p=0.555) in the PFC (FIG. 32A-32C).

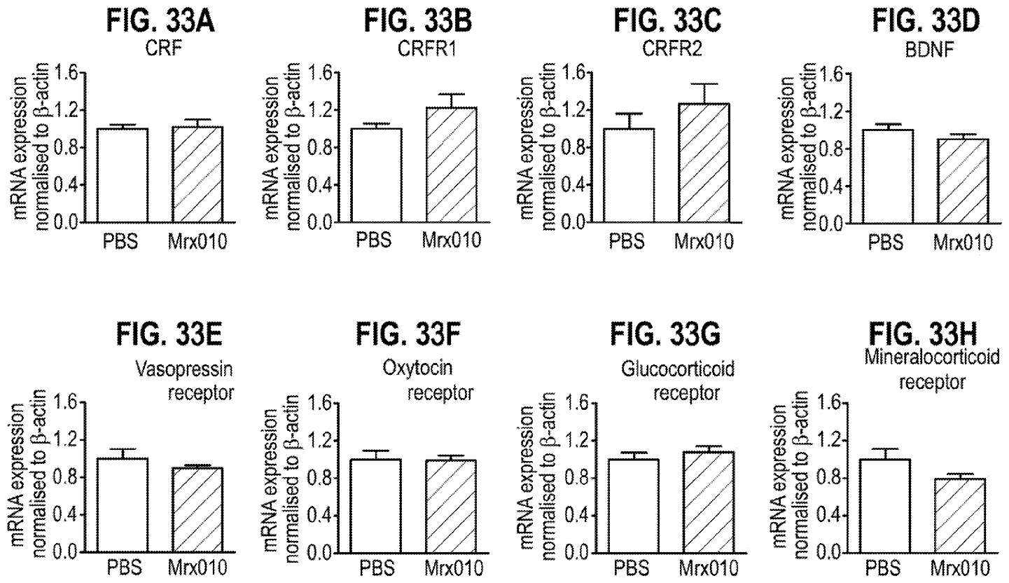

MRX010 did not show an appreciable effect on mRNA expression for endocrine markers mRNA expression of vasopressin receptor (t(12)=0.843, p=0.416), CRF (t(12)=0.239, p=0.815), CRFR1 (t(12)=0.159, p=0.138), CRFR2 (t(11)=1.014, p=0.332), BDNF (t(12)=1.133, p=0.280), oxytocin receptor (t(12)=0.081, p=0.370), glucocorticoid receptor (t(12)=0.749, p=0.468), mineralocorticoid receptor (t(12)=1.473, p=0.166) in hippocampus (FIG. 33A=33H).

MRX010 did not show an appreciable effect on mRNA expression of amygdala endocrine markers CRFR1 (t(12)=0.164, p=0.827), CRFR2 (t(11)=0.288, p=0.4779), vasopressin receptor (t(12)=0.013, p=0.990), oxytocin receptor (t(11)=0.787, p=0.488), glucocorticoid receptor (t(11)=0.444, p=0.665), mineralocorticoid receptor (t(10)=1.023, p=0.331) but demonstrated decreased mRNA expression for BDNF (t(11)=2.943, p=0.013) (FIG. 34A-34G).

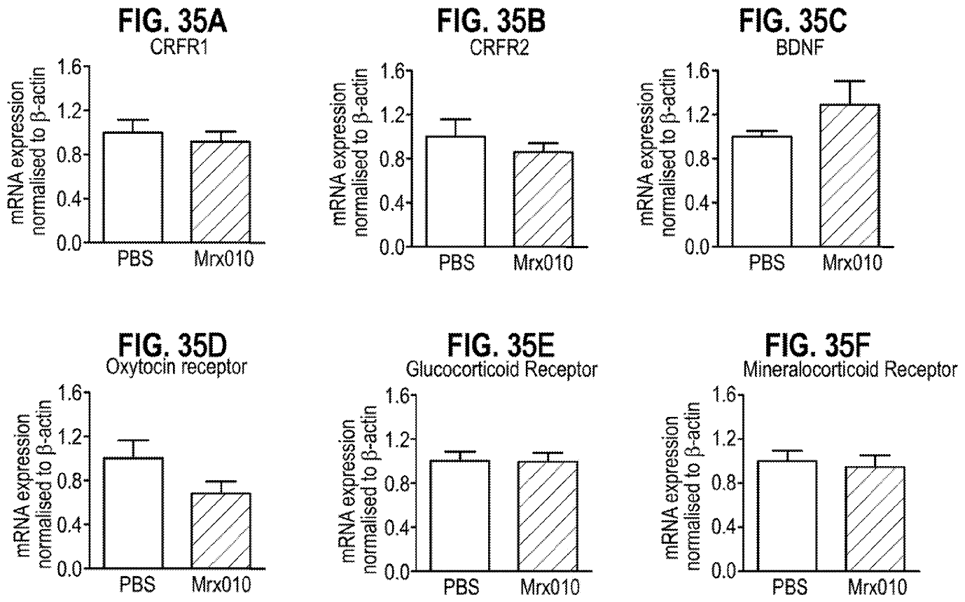

MRX010 did not show an appreciable effect on mRNA expression of PFC endocrine markers CRFR1 (t(12)=0.531, p=0.605), CRFR2 (t(12)=0.71, p=0.492), BDNF (t(11)=1.391, p=0.192), oxytocin receptor (t(11)=1.545, p=0.151), glucocorticoid receptor (t(12)=0.044, p=0.965), mineralocorticoid receptor (t(11)=0.381, p=0.710) (FIG. 35A-35F).

### EXAMPLE 6c

### Discussion of Preclinical Study

The effect of chronic administration of the live biotherapeutic MRX010 on a number of readouts including gut permeability and function, metabolic profile (SCFAs), systemic immune activation (splenocyte assay), plasma levels of amino acids, central neurotransmitter release and gene expression for inflammatory, endocrine and neurotransmitter markers in the hippocampus, amygdala and prefrontal cortex was assessed in this preclinical study.

MRX010 showed no observable effect on ileum or colon permeability, nor the expression of tight junction proteins associated with maintaining barrier integrity. The finding that the chronic treatment with MRX010 does not alter permeability suggests that it does not negatively impact on gut permeability and integrity. MRX010 also did not alter IDO-1 nor TPH1 suggesting that it does not alter serotonin production nor the tryptophan/kynurenine pathway in the gut.

MRX010 administration did not cause any observable effects on caecal SCFA production. This suggests that the chronic regime of MRX010 did not alter the fermentation, or the bacteria responsible for the fermentation of non-digestible fibres from the diet.

MRX010 increased the expression of the pro-inflammatory cytokine IL-1β in splenocytes in response to the viral mimetic (concavalin A) challenge. MRX010 had no further observable effects on the range of cytokines studied in response to LPS or concavalin A. This demonstrates that MRX010 administration does not appear to exert profound influence on the innate peripheral immune response.

Plasma levels of amino acids were largely unaltered following chronic administration of MRX010. There are nine essential amino acids that cannot be synthesised de novo and must be supplied directly in the diet or by breakdown of the diet. These include valine, phenylalanine, threonine, tryptophan, methionine, leucine, isoleucine, lysine, and histidine. Six other amino acids are considered conditionally essential in the human diet, meaning their synthesis can be limited under special pathophysiological conditions, such as prematurity in the infant or individuals in severe catabolic distress. These six amino acids include arginine, cysteine, glycine, glutamine, proline, and tyrosine. Five amino acids are dispensable in humans, meaning they can be synthesized in sufficient quantities in the body. These five are alanine, aspartic acid, asparagine, glutamic acid and serine.

However, the levels of proline were decreased following MRX010 administration, suggesting that this bacterium may play a role in metabolism of key amino acids from the diet.

Neurotransmitter levels in the brainstem were similarly unaltered following chronic MRX010 administration, suggesting that this live biotherapeutic does not negatively impact on behaviours that are governed by monoamine levels at the level of the brainstem.

Finally, central gene expression for inflammatory, endocrine and neurotransmitter receptors were, for the most part, unaltered following chronic MRX010 administration. Only NMDA receptor 2B and BDNF mRNA expression in the amygdala were increased while there were no changes in hippocampus or prefrontal cortex. These data taken together with the many other outputs, confirm that MRX010 administration at this concentration for this dosing regime does not negatively impact on systemic and central physiological events. From a translational perspective, these data suggest that this live biotherapeutic may have a high tolerability profile with minimal non-desirable side-effects.

| Sequences | |
|---|---|
| (Enterococcus faecium 16S rRNA gene, strain LMG 11423-AJ301830) | |
| | SEQ ID NO: 1 |
| 1 | agagtttgat cctggctcag gacgaacgct ggcggcgtgc ctatacatgc aagtcgaacg |
| 61 | cttctttttc caccggagct tgctccaccg gaaaaagagg agtggcgaac gggtgagtaa |
| 121 | cacgtgggta acctgcccat cagaaaggga taacacttgg aaacaggtgc taataccgta |
| 181 | taacaaatca aaaccgcatg gttttgattt gaaaggcgct ttcgggtgtc gctgatggat |
| 241 | ggacccgcgg tgcattagct agttggtgag gtaacggctc accaaggcca cgatgcatag |
| 301 | ccgcacctga gagggtgatc ggccacattg ggactgagac acggcccaaa ctctacggga |
| 361 | ggcagcagta gggaatcttc ggcaatggac gaaagtctga ccgagcaacg ccgcgtgagt |
| 421 | gaagaaggtt ttcggatcgt aaaactctgt tgttagagaa gaacaaggat gagagtaact |
| 481 | gttcatccct tgacggtatc taaccagaaa gccacggcta actacgtgcc agcagccgcg |
| 541 | gtaatacgta ggtggcaagc gttgtccgga tttattgggc gtaaagcgag cgcaggcggt |
| 601 | tcttaagtct gatgtgaaag cccccggctc aaccggggag ggtcattgga aactgggaga |
| 661 | cttgagtgca gaagaggaga gtggaattcc atgtgtagcg gtgaaatgcg tagatatatg |
| 721 | gaggaacacc agtggcgaag gcggctctct ggtctgtaac tgacgctgag gctcgaaagc |
| 781 | gtggggagca aacaggatta gataccctgg tagtccacgc cgtaaacgat gagtgctaag |
| 841 | tgttggaggg tttccgccct tcagtgctgc agctaacgca ttaagcactc cgcctgggga |
| 901 | gtacgaccgc aaggttgaaa ctcaaaggaa ttgacggggg cccgcacaag cggtggagca |
| 961 | tgtggtttaa ttcgaagcaa cacgaagaac cttaccaggt cttgacatcc tttgaccact |
| 1021 | ctagagatag agcttcccct tcgggggcaa agtgacaggt ggtgcatggt tgtcgtcagc |
| 1081 | tcgtgtcgtg agatgttggg ttaagtcccg caacgagcgc aacccttatt gttagttgcc |
| 1141 | atcattcagt tgggcactct agcaagactg ccggtgacaa accggaggaa ggtggggatg |
| 1201 | acgtcaaatc atcatgcccc ttatgacctg ggctacacac gtgctacaat gggaagtaca |
| 1261 | acgagttgcg aagtcgcgag gctaagctaa tctcttaaag cttctctcag ttcggattgc |
| 1321 | aggctgcaac tcgcctgcat gaagccggaa tcgctagtaa tcgcggatca gcacgccgcg |
| 1381 | tgaatacgtt cccgggcctt gtacacaccg cccgtcacac cacgagagtt tgtaacaccc |
| 1441 | gaagtcggtg aggtaacctt ttggagccag ccgcctaagg tgggatagat gattggggtg |
| 1501 | aagtcgtaac aaggtagccg tatctgaagg tgcggctgga tcacctcctt tctaaggaat |
| 1561 | attacggata ctacacactt tttttttact tttttcattt tgaatttact ctcaaacact |
| 1621 | tttgttcatt gacactgcat atctgaagta t |

(consensus 16S rRNA sequence for Enterococcus faecium strain MRX010)
SEQ ID NO: 2
TTAGGCGGCTGGCTCCAAAAGGTTACCTCACCGACTTCGGGTGTTACAAACTCTCGTGGT
GTGACGGGCGGTGTGTACAAGGCCCGGGAACGTATTCACCGCGGCGTGCTGATCCGCGA
TTACTAGCGATTCCGGCTTCATGCAGGCGAGTTGCAGCCTGCAATCCGAACTGAGAGAA
GCTTTAAGAGATTAGCTTAGCCTCGCGACTTCGCAACTCGTTGTACTTCCCATTGTAGCA
CGTGTGTAGCCCAGGTCATAAGGGGCATGATGATTTGACGTCATCCCCACCTTCCTCCGG
TTTGTCACCGGCAGTCTTGCTAGAGTGCCCAACTGAATGATGGCAACTAACAATAAGGG
TTGCGCTCGTTGCGGGACTTAACCCAACATCTCACGACACGAGCTGACGACAACCATGC
ACCACCTGTCACTTTGCCCCCGAAGGGGAAGCTCTATCTCTAGAGTGGTCAAAGGATGT
CAAGACCTGGTAAGGTTCTTCGCGTTGCTTCGAATTAAACCACATGCTCCACCGCTTGTG
CGGGCCCCCGTCAATTCCTTTGAGTTTCAACCTTGCGGTCGTACTCCCCAGGCGGAGTGC
TTAATGCGTTAGCTGCAGCACTGAAGGGCGGAAACCCTCCAACACTTAGCACTCATCGT
TTACGGCGTGGACTACCAGGGTATCTAATCCTGTTTGCTCCCCACGCTTTCGAGCCTCAG
CGTCAGTTACAGACCAGAGAGCCGCCTTCGCCACTGGTGTTCCTCCATATATCTACGCAT
TTCACCGCTACACATGGAATTCCACTCTCCTCTTCTGCACTCAAGTCTCCCAGTTTCCAAT
GACCCTCCCCGGTTGAGCCGGGGGCTTTCACATCAGACTTAAGAAACCGCCTGCGCTCG
CTTTACGCCCAATAAATCCGGACAACGCTTGCCACCTACGTATTACCGCGGCTGCTGGC
ACGTAGTTAGCCGTGGCTTTCTGGTTAGATACCGTCAAGGGATGAACAGTTACTCTCATC
CTTGTTCTTCTCTAACAACAGAGTTTTACGATCCGAAAACCTTCTTCACTCACGCGGCGT
TGCTCGGTCAGACTTTCGTCCATTGCCGAAGATTCCCTACTGCTGCCTCCCGTAGGAGTT
TGGGCCGTGTCTCAGTCCCAATGTGGCCGATCACCCTCTCAGGTCGGCTATGCATCGTGG
CCTTGGTGAGCCGTTACCTCACCAACTAGCTAATGCACCGCGGGTCCATCCATCAGCGA
CACCCGAAAGCGCCTTTCAAATCAAAACCATGCGGTTTTGATTGTTATACGGTATTAGCA
CCTGTTTCCAAGTGTTATCCCCTTCTGATGGGCAGGTTACCCACGTGTTACTCACCCGTT
CGCCACTCCTCTTTTTCCGGTGGAGCAAGCTCCGGTGGAAAAAGAAGCGTTCGACTGCA
SEQ ID NO: 3 (chromosome sequence of Enterococcus faecium strain
DO)-see electronic sequence listing.
SEQ ID NO: 4 (plasmid 1 sequence of Enterococcus faecium strain
DO)-see electronic sequence listing.
SEQ ID NO: 5 (plasmid 2 sequence of Enterococcus faecium strain
DO)-see electronic sequence listing.
SEQ ID NO: 6 (plasmid 3 sequence of Enterococcus faecium strain
DO)-see electronic sequence listing.

### REFERENCES

[1] Spor et al. (2011) Nat Rev Microbiol. 9(4):279-90.
[2] Eckburg et al. (2005) Science. 10;308(5728):1635-8.
[3] Macpherson et al. (2001) Microbes Infect. 3(12):1021-35
[4] Macpherson et al. (2002) Cell Mol Life Sci. 59(12):2088-96.
[5] Mazmanian et al. (2005) Cell 15;122(1):107-18.
[6] Frank et al. (2007) PNAS 104(34):13780-5.
[7] Scanlan et al. (2006) J Clin Microbiol. 44(11):3980-8.
[8] Kang et al. (2010) Inflamm Bowel Dis. 16(12):2034-42.
[9] Machiels et al. (2013) Gut. 63(8):1275-83.
[10] Mayer et al (2014) The Journal of Neuroscience 34(46):15490-15496
[11] Cryan and Dinan (2015) Neuropsychopharmacology, 40: 241-2.
[12] Zhou and Foster (2015) Neuropsychiatric Disease and Treatment 11: 715-723.
[13] Wang and Kasper (2014) Brain Behav Immun. 38: 1-12.
[14] WO 2013/050792
[15] WO 03/046580
[16] WO 2013/008039
[17] WO 2014/167338
[18] Goldin and Gorbach (2008) Clin Infect Dis. 46 Suppl 2:S96-100.
[19] Azad et al. (2013) BMJ. 347:f6471.
[20] Bravo et al. (2011) Proc Natl Acad Sci USA, 108: 16050-5.
[21] Kantak et al. (2014) Behav Pharmacol. 25: 71-9.
[22] Savignac et al. (2014) Neurogastroenterol Motil. 26: 1615-27.
[23] WO 2016/069795
[24] US 2016/120920
[25] Huycke et al. (1998) Emerging Infectious Diseases 4(2): 239-249.
[26] de Theije et al. (2014) Brain Behav Immun. 37: 197-206.
[27] Meyza and Blanchard (2017) Neurosci Biobehav Rev.
[28] Schleifer and Kilpper-Bälz (1984) Int J Syst Evol Microbiol. 34: 31-34.
[29] İspirli et al. (2015) Can J Microbiol. 61(11):861-70.
[30] Divyashri et al. (2015) J Med Microbiol. doi: 10.1099/jmm.0.000184.
[31] Lauková et al. (2105) Folia Microbiol (Praha). Sep 9.
[32] Büsing and Zeyner (2015) Benef Microbes. 6(1):41-4.
[33] Schmitz et al. (2015) J Vet Intern Med. 29(2):533-43.
[34] Mansour et al. (2014) Microbiol Immunol. 58(10):559-69.
[35] Masco et al. (2003) Systematic and Applied Microbiology, 26:557-563.
[36] Srůtková et al. (2011) J. Microbiol. Methods, 87(1):10-6.
[37] GIDEON Guide to Medically Important Bacteria (2017) GIDEON Informatics, Inc.
[38] Wang et al. (2016) J Neurogasfroenterol Motil 22: 589-605.
[39] Li and Zhou (2016) Neuroscience 324: 131-139.
Vorstman et al. (2009) Neuropsychopharmacology; 34(3): 739-746.
[41] Carlson (2012) Pharmacol. Biochem. Behay.; 100(4): 850-854.
[42] Kang and Kim (2015) Front. Mol. Neurosci.; 8(17) 1-9.
[43] Zheng et al. (2016) Scientific Reports; 6 (31241) 1-8.
[44] Miyamoto-Shinohara et al. (2008) J. Gen. Appl. Microbiol., 54,9-24.
[45] Cryopreservation and Freeze-Drying Protocols, ed. by Day and McLellan, Humana Press.
[46] Leslie et al. (1995) Appl. Environ. Microbiol. 61, 3592-3597.
[47] Mitropoulou et al. (2013) J Nutr Metab. (2013) 716861.
[48] Kailasapathy et al. (2002) Curr Issues Intest Microbiol. 3(2):39-48.
[49] Handbook of Pharmaceutical Excipients, 2nd Edition, (1994), Edited by A Wade and P J Weller
[50] Remington's Pharmaceutical Sciences, Mack Publishing Co. (A. R. Gennaro edit. 1985)
[51] US 2016/0067188
[52] Handbook of Microbiological Media, Fourth Edition (2010) Ronald Atlas, CRC Press.
[53] Maintaining Cultures for Biotechnology and Industry (1996) Jennie C. Hunter-Cevera, Academic Press
[54] Strobel (2009) Methods Mol Biol. 581:247-61.
[55] Gennaro (2000) Remington: The Science and Practice of Pharmacy. 20th edition, ISBN: 0683306472.
[56] Molecular Biology Techniques: An Intensive Laboratory Course, (Ream et al., eds., 1998, Academic Press).
[57] Methods In Enzymology (S. Colowick and N. Kaplan, eds., Academic Press, Inc.)
[58] Handbook of Experimental Immunology, Vols. I-IV (D. M. Weir and C. C. Blackwell, eds, 1986, Blackwell Scientific Publications)
[59] Sambrook et al. (2001) Molecular Cloning: A Laboratory Manual, 3rd edition (Cold Spring Harbor Laboratory Press).
[60] Handbook of Surface and Colloidal Chemistry (Birdi, K. S. ed., CRC Press, 1997)
[61] Ausubel et al. (eds) (2002) Short protocols in molecular biology, 5th edition (Current Protocols).
[62] PCR (Introduction to Biotechniques Series), 2nd ed. (Newton & Graham eds., 1997, Springer Verlag)
[63] Current Protocols in Molecular Biology (F. M. Ausubel et al., eds., 1987) Supplement 30
[64] Smith & Waterman (1981) Adv. Appl. Math. 2: 482-489.
[65] Crawley (2012) Dialogues Clin Neurosci. 14: 293-305.
[66] Hsiao et al. (2013) Cell 155(7): 1451-1463.
[67] Desbonnet, Clarke et al. (2015) Brain, Behavior, and Immunity 48: 165-173
[68] Cryan and Mombereau (2004) Mol Psychiatry 9: 326-57.

1.-32. (canceled)

33. A method of treating a neurodevelopmental disorder or a neuropsychiatric condition in a subject in need thereof, comprising administering to the subject a pharmaceutical composition that comprises a therapeutically effective amount of a bacteria strain of the species Enterococcus faecium, wherein the Enterococcus faecium bacteria strain comprises a polynucleotide sequence of a 16S rRNA gene that has at least 95% sequence identity to the polynucleotide sequence of SEQ ID NO:2, as determined by a Smith-Waterman homology search algorithm using an affine gap search with a gap open penalty of 12, a gap extension penalty of 2, and a Blocks Substitution Matrix (BLOSUM) of 62, and wherein the administering treats the neurodevelopmental disorder or the neuropsychiatric condition in the subject.

34. The method of claim 33, wherein the therapeutically effective amount of the Enterococcus faecium bacteria strain comprises at least 1×10³ CFU/g of the Enterococcus faecium bacteria strain with respect to a total weight of the pharmaceutical composition.

35. The method of claim 33, wherein the therapeutically effective amount of the Enterococcus faecium bacteria strain comprises from about 1×10⁶ to about 1×10¹¹ CFU/g of the Enterococcus faecium bacteria strain with respect to a total weight of the pharmaceutical composition.

36. The method of claim 33, wherein the pharmaceutical composition only contains the Enterococcus faecium bacteria strain.

37. The method of claim 33, wherein the subject has the neurodevelopmental disorder, and wherein the neurodevelopmental disorder is an autism spectrum disorder (ASD) or a child developmental disorder.

38. The method of claim 37, wherein the subject has the ASD, and wherein the ASD is autism or Asperger Syndrome.

39. The method of claim 33, wherein the subject has the neuropsychiatric condition, and wherein the neuropsychiatric condition is selected from the group consisting of obsessive compulsive disorder (OCD), major depressive disorder, seasonal affective disorder, an anxiety disorder, chronic fatigue syndrome, post-traumatic stress disorder, a schizophrenia spectrum disorder, bipolar disorder, psychosis, and mood disorder.

40. The method of claim 39, wherein the subject has the schizophrenia spectrum disorder, and wherein the schizophrenia spectrum disorder is schizophrenia.

41. The method of claim 39, wherein the subject has the major anxiety disorder, and wherein the major anxiety disorder is selected from the group consisting of generalized anxiety disorder (GAD), specific phobia, social anxiety disorder, separation anxiety disorder, agoraphobia, panic disorder, and selective mutism.

42. The method of claim 33, wherein the Enterococcus faecium bacteria strain is live and capable of at least partially colonizing an intestine of the subject.

43. The method of claim 33, further comprising administering an additional therapeutic agent.

44. The method of claim 33, wherein said administering comprises oral, rectal, nasal, buccal, sublingual, or subcutaneous administration.

45. The method of claim 33, wherein the pharmaceutical composition is formulated for delivery to an intestine of the subject.

46. The method of claim 33, wherein the Enterococcus faecium bacteria strain comprises a polynucleotide sequence of a 16S rRNA gene that has at least 99% sequence identity to the polynucleotide sequence of SEQ ID NO:2, as determined by a Smith-Waterman homology search algorithm using an affine gap search with a gap open penalty of 12, a gap extension penalty of 2, and a Blocks Substitution Matrix (BLOSUM) of 62.

47. The method of claim 33, wherein the Enterococcus faecium bacteria strain comprises the polynucleotide sequence of SEQ ID NO:2.

48. The method of claim 33, wherein the Enterococcus faecium bacteria strain is the bacteria strain deposited under accession number NCIMB 42487.

49. The method of claim 33, wherein the subject is human.

50. A method of treating a behavioral or psychiatric condition in a subject in need thereof, comprising administering to the subject a pharmaceutical composition that comprises a therapeutically effective amount of a bacteria strain of the species Enterococcus faecium, wherein the Enterococcus faecium bacteria strain comprises a polynucleotide sequence of a 16S rRNA gene that has at least 95% sequence identity to the polynucleotide sequence of SEQ ID NO:2, as determined by a Smith-Waterman homology search algorithm using an affine gap search with a gap open penalty of 12, a gap extension penalty of 2, and a Blocks Substitution Matrix (BLOSUM) of 62, and wherein the administering treats the behavioral or psychiatric condition in the subject.

51. The method of claim 50, wherein the therapeutically effective amount of the Enterococcus faecium bacteria strain comprises at least 1×10³ CFU/g of the Enterococcus faecium bacteria strain with respect to a total weight of the pharmaceutical composition.

52. The method of claim 50, wherein the therapeutically effective amount of the Enterococcus faecium bacteria strain comprises from about 1×10⁶ to about 1×10¹¹ CFU/g of the Enterococcus faecium bacteria strain with respect to a total weight of the pharmaceutical composition.

53. The method of claim 50, wherein the behavioral or psychiatric condition comprises a repetitive behavior, a compulsive behavior, or an anxious behavior.

54. The method of claim 50, wherein the bacteria strain comprises a 16S rRNA gene sequence that has at least 99% sequence identity to the polynucleotide sequence of SEQ ID NO:2, as determined by a Smith-Waterman homology search algorithm using an affine gap search with a gap open penalty of 12, a gap extension penalty of 2, and a Blocks Substitution Matrix (BLOSUM) of 62.

55. The method of claim 50, wherein the Enterococcus faecium bacteria strain is live and capable of at least partially colonizing an intestine of the subject.

56. The method of claim 50, wherein the Enterococcus faecium bacteria strain comprises the polynucleotide sequence of SEQ ID NO:2.

57. The method of claim 50, wherein the Enterococcus faecium bacteria strain is the bacteria strain deposited under accession number NCIMB 42487.

58. The method of claim 50, wherein the subject is human.

59. A method of treating a behavioral or psychiatric condition in a subject suffering from a neurocognitive disorder, comprising administering to the subject a pharmaceutical composition that comprises a therapeutically effective amount of a bacteria strain of the species Enterococcus faecium, wherein the Enterococcus faecium bacteria strain comprises a polynucleotide sequence of a 16S rRNA gene that has at least 99% sequence identity to the polynucleotide sequence of SEQ ID NO:2, as determined by a Smith-Waterman homology search algorithm using an affine gap search with a gap open penalty of 12, a gap extension penalty of 2, and a Blocks Substitution Matrix (BLOSUM) of 62, wherein the administering treats the behavioral or psychiatric condition in a subject suffering from the neurocognitive disorder.

60. The method of claim 59, wherein the neurocognitive disorder is selected from the group consisting of Alzheimer's disease, vascular dementias, Lewy body disease, frontotemporal dementia, Parkinson's disease, Creutzfeldt-Jakob disease, Huntington's disease, and Wernicke-Korsakoff syndrome.

61. The method of claim 59, wherein the therapeutically effective amount of the Enterococcus faecium bacteria strain comprises at least 1×10³ CFU/g of the Enterococcus faecium bacteria strain with respect to a total weight of the pharmaceutical composition.

62. The method of claim 59, wherein the therapeutically effective amount of the Enterococcus faecium bacteria strain comprises from about 1×10⁶ to about 1×10¹¹ CFU/g of the Enterococcus faecium bacteria strain with respect to a total weight of the pharmaceutical composition.

63. The method of claim 59, wherein the behavioral or psychiatric condition comprises a repetitive behavior, a compulsive behavior, or an anxious behavior.

64. The method of claim 59, wherein the bacteria strain comprises a 16S rRNA gene sequence that has at least 99% sequence identity to the polynucleotide sequence of SEQ ID NO:2, as determined by a Smith-Waterman homology search algorithm using an affine gap search with a gap open penalty of 12, a gap extension penalty of 2, and a Blocks Substitution Matrix (BLOSUM) of 62.

65. The method of claim 59, wherein the Enterococcus faecium bacteria strain comprises the polynucleotide sequence of SEQ ID NO:2.

66. The method of claim 59, wherein the Enterococcus faecium bacteria strain is the bacteria strain deposited under accession number NCIMB 42487.

67. The method of claim 59, wherein the subject is human.
